SEQUENCE LISTING (30 sequences)

SEQ ID NO: 1 - DNA, 1509 bp, Homo sapiens
  source 1..1509: mol_type = genomic DNA
atggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactgtaaagtcatcacctggccttggtcctgtggaactggcagctgtcattgctggaccagtgtgcttcgtctgcatctcactcatgttgatggtctatatctgccacaaccgcactgtcattcaccatcgagtgccaaatgaagaggacccttcattagatcgcccttttatttcagagggtactacgttgaaagacttaatttatgatatgacaacgtcaggttctggctcaggtttaccattgcttgttcagagaacaattgcgagaactattgtgttacaagaaagcattggcaaaggtcgatttggagaagtttggagaggaaagtggcggggagaagaagttgctgttaagatattctcctctagagaagaacgttcgtggttccgtgaggcagagatttatcaaactgtaatgttacgtcatgaaaacatcctgggatttatagcagcagacaataaagacaatggtacttggactcagctctggttggtgtcagattatcatgagcatggatccctttttgattacttaaacagatacacagttactgtggaaggaatgataaaacttgctctgtccacggcgagcggtcttgcccatcttcacatggagattgttggtacccaaggaaagccagccattgctcatagagatttgaaatcaaagaatatcttggtaaagaagaatggaacttgctgtattgcagacttaggactggcagtaagacatgattcagccacagataccattgatattgctccaaaccacagagtgggaacaaaaaggtacatggcccctgaagttctcgatgattccataaatatgaaacattttgaatccttcaaacgtgctgacatctatgcaatgggcttagtattctgggaaattgctcgacgatgttccattggtggaattcatgaagattaccaactgccttattatgatcttgtaccttctgacccatcagttgaagaaatgagaaaagttgtttgtgaacagaagttaaggccaaatatcccaaacagatggcagagctgtgaagccttgagagtaatggctaaaattatgagagaatgttggtatgccaatggagcagctaggcttacagcattgcggattaagaaaacattatcgcaactcagtcaacaggaaggcatcaaaatg

SEQ ID NO: 2 - DNA, 1509 bp, synthetic construct
  source 1..1509: mol_type = other DNA
atggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactgtaaagtcatcacctggccttggtcctgtggaactggcagctgtcattgctggaccagtgtgcttcgtctgcatctcactcatgttgatggtctatatctgccacaaccgcactgtcattcaccatcgagtgccaaatgaagaggacccttcattagatcgcccttttatttcagagggtactacgttgaaagacttaatttatgatatgacaacgtcaggttctggctcaggtttaccattgcttgttcagagaacaattgcgagagatattgtgttacaagaaagcattggcaaaggtcgatttggagaagtttggagaggaaagtggcggggagaagaagttgctgttaagatattctcctctagagaagaacgttcgtggttccgtgaggcagagatttatcaaactgtaatgttacgtcatgaaaacatcctgggatttatagcagcagacaataaagacaatggtacttggactcagctctggttggtgtcagattatcatgagcatggatccctttttgattacttaaacagatacacagttactgtggaaggaatgataaaacttgctctgtccacggcgagcggtcttgcccatcttcacatggagattgttggtacccaaggaaagccagccattgctcatagagatttgaaatcaaagaatatcttggtaaagaagaatggaacttgctgtattgcagacttaggactggcagtaagacatgattcagccacagataccattgatattgctccaaaccacagagtgggaacaaaaaggtacatggcccctgaagttctcgatgattccataaatatgaaacattttgaatccttcaaacgtgctgacatctatgcaatgggcttagtattctgggaaattgctcgacgatgttccattggtggaattcatgaagattaccaactgccttattatgatcttgtaccttctgacccatcagttgaagaaatgagaaaagttgtttgtgaacagaagttaaggccaaatatcccaaacagatggcagagctgtgaagccttgagagtaatggctaaaattatgagagaatgttggtatgccaatggagcagctaggcttacagcattgcggattaagaaaacattatcgcaactcagtcaacaggaaggcatcaaaatg

SEQ ID NO: 3 - DNA, 1509 bp, synthetic construct
  source 1..1509: mol_type = other DNA
atggaagcagcagtggcagctcctagacccaggctcttgcttctggttctggcagccgctgctgcagctgctgccgcattgctgccaggagcaacagctctgcaatgtttctgtcacctgtgtaccaaggacaatttcacatgcgtgacagacggactgtgcttcgtcagtgtgactgagaccaccgacaaagtcattcataactccatgtgtattgcagagatcgatctcattccacgcgatagaccctttgtgtgtgctccaagttctaagaccggctccgttaccacaacatactgctgtaaccaggatcactgcaataaaattgaacttcccactacagtcaaatcatctccaggactgggtcccgtcgaacttgctgcagtgattgcaggacccgtctgtttcgtgtgtatcagcctgatgctcatggtttatatttgtcacaatcgcaccgtgattcaccatcgcgtccctaatgaagaggacccatccctcgataggccattcatctcggagggtacaaccctgaaagacctgatttatgatatgactacttctggatctggtagtgggttgccccttctcgtgcagaggactatcgcacgggatatcgtgctgcaagaatctatcggtaagggacggttcggtgaggtctggagaggaaagtggaggggtgaagaggttgcagtcaagatcttttcttccagggaagaacggagctggttcagagaggctgaaatctatcagactgtcatgcttagacacgagaacatcctgggctttattgccgccgacaataaagataatggcacctggacccaactgtggttggtgtctgactatcacgaacatgggagcctgttcgattatttgaataggtacacagtgactgtcgagggcatgattaaactggctctgagcacagctagcggccttgctcacttgcacatggagatcgtcgggacccaagggaagcctgcgattgcccacagagatctgaaatccaagaatatactggtgaagaaaaatgggacttgttgcattgctgacttgggtctggcggtgagacatgactcagccacagataccattgacatcgccccaaaccacagggttgggacaaagaggtacatggcccctgaggtccttgacgactccattaatatgaaacatttcgagtccttcaagcgcgcggatatttatgctatgggacttgtgttttgggagattgccagacggtgttcaatcggagggattcatgaggactaccagttgccctattacgacctggtgcctagtgatccctctgtggaggagatgagaaaggtggtctgtgaacagaagctcagacctaatatccccaaccggtggcagtcatgcgaagccctgagagtgatggccaaaatcatgcgtgaatgctggtatgcaaatggtgccgctagacttactgccctccgtattaaaaaaaccctcagccaactgtctcagcaagagggaataaagatg

SEQ ID NO: 4 - DNA, 1401 bp, Homo sapiens
  source 1..1401: mol_type = genomic DNA
atgtcgtccatcttgccattcacgccgccagttgtgaagagactgctgggatggaagaagtcagctggtgggtctggaggagcaggcggaggagagcagaatgggcaggaagaaaagtggtgtgagaaagcagtgaaaagtctggtgaagaagctaaagaaaacaggacgattagatgagcttgagaaagccatcaccactcaaaactgtaatactaaatgtgttaccataccaagcacttgctctgaaatttggggactgagtacaccaaatacgatagatcagtgggatacaacaggcctttacagcttctctgaacaaaccaggtctcttgatggtcgtctccaggtatcccatcgaaaaggattgccacatgttatatattgccgattatggcgctggcctgatcttcacagtcatcatgaactcaaggcaattgaaaactgcgaatatgcttttaatcttaaaaaggatgaagtatgtgtaaacccttaccactatcagagagttgagacaccagttttgcctccagtattagtgccccgacacaccgagatcctaacagaacttccgcctctggatgactatactcactccattccagaaaacactaacttcccagcaggaattgagccacagagtaattatattccagaaacgccacctcctggatatatcagtgaagatggagaaacaagtgaccaacagttgaatcaaagtatggacacaggctctccagcagaactatctcctactactctttcccctgttaatcatagcttggatttacagccagttacttactcagaacctgcattttggtgttcgatagcatattatgaattaaatcagagggttggagaaaccttccatgcatcacagccctcactcactgtagatggctttacagacccatcaaattcagagaggttctgcttaggtttactctccaatgttaaccgaaatgccacggtagaaatgacaagaaggcatataggaagaggagtgcgcttatactacataggtggggaagtttttgctgagtgcctaagtgatagtgcaatctttgtgcagagccccaattgtaatcagagatatggctggcaccctgcaacagtgtgtaaaattccaccaggctgtaatctgaagatcttcaacaaccaggaatttgctgctcttctggctcagtctgttaatcagggttttgaagccgtctatcagctaactagaatgtgcaccataagaatgagttttgtgaaagggtggggagcagaataccgaaggcagacggtaacaagtactccttgctggattgaacttcatctgaatggacctctacagtggttggacaaagtattaactcagatgggatccccttcagtgcgttgctcaagcatgtca

SEQ ID NO: 5 - DNA, 1401 bp, synthetic construct
  source 1..1401: mol_type = other DNA
atgtcgtccatcttgccattcacgccgccagttgtgaagagactgctgggatggaagaagtcagctggtgggtctggaggagcaggcggaggagagcagaatgggcaggaagaaaagtggtgtgagaaagcagtgaaaagtctggtgaagaagctaaagaaaacaggacgattagatgagcttgagaaagccatcaccactcaaaactgtaatactaaatgtgttaccataccaagcacttgctctgaaatttggggactgagtacaccaaatacgatagatcagtgggatacaacaggcctttacagcttctctgaacaaaccaggtctcttgatggtcgtctccaggtatcccatcgaaaaggattgccacatgttatatattgccgattatggcgctggcctgatcttcacagtcatcatgaactcaaggcaattgaaaactgcgaatatgcttttaatcttaaaaaggatgaagtatgtgtaaacccttaccactatcagagagttgagacaccagttttgcctccagtattagtgccccgacacaccgagatcctaacagaacttccgcctctggatgactatactcactccattccagaaaacactaacttcccagcaggaattgagccacagagtaattatattccagaaacgccacctcctggatatatcagtgaagatggagaaacaagtgaccaacagttgaatcaaagtatggacacaggctctccagcagaactatctcctactactctttcccctgttaatcatagcttggatttacagccagttacttactcagaacctgcattttggtgttcgatagcatattatgaattaaatcagagggttggagaaaccttccatgcatcacagccctcactcactgtagatggctttacagacccatcaaattcagagaggttctgcttaggtttactctccaatgttaaccgaaatgccacggtagaaatgacaagaaggcatataggaagaggagtgcgcttatactacataggtggggaagtttttgctgagtgcctaagtgatagtgcaatctttgtgcagagccccaattgtaatcagagatatggctggcaccctgcaacagtgtgtaaaattccaccaggctgtaatctgaagatcttcaacaaccaggaatttgctgctcttctggctcagtctgttaatcagggttttgaagccgtctatcagctaactagaatgtgcaccataagaatgagttttgtgaaagggtggggagcagaataccgaaggcagacggtaacaagtactccttgctggattgaacttcatctgaatggacctctacagtggttggacaaagtattaactcagatgggatccccttcagtgcgttgcgaagagatggaa

SEQ ID NO: 6 - DNA, 1401 bp, synthetic construct
  source 1..1401: mol_type = other DNA
atgagctctatcctccccttcacacctcctgttgtgaaacggctcttgggatggaagaaaagcgcaggtggctctggtggagctggaggcggagagcaaaacgggcaggaagagaaatggtgcgagaaagcagtgaagtctctggtcaagaaactcaagaagacaggaagactggacgaacttgagaaagccatcacaactcaaaactgtaataccaaatgcgttactatcccctcaacttgttcagagatctggggtctgagcacaccaaataccattgaccagtgggacaccactggactgtatagcttcagcgagcaaactcgcagccttgatggaaggctccaagtgtcccatagaaaggggctaccacatgtgatttactgtcggctttggaggtggcctgatctgcactctcatcacgaactgaaggcgatcgagaactgcgaatacgcgtttaacctcaagaaagacgaagtgtgtgtcaatccctaccattaccaacgtgttgagacccctgtactgcctccagtgcttgtgcccagacacaccgaaattctgactgagttgccacctttggatgattacacacattccatccccgagaatactaacttcccagcaggtatcgaacctcagtccaactacatccccgaaacaccacctcctggatatatcagcgaagatggcgagacatctgaccaacagctcaatcagagcatggatacaggatctcccgctgaactcagccctacaactctcagccctgttaaccacagccttgatctgcaaccagtgacttatagtgaacctgcattctggtgctccattgcatactacgagctgaatcagagggttggcgagacatttcacgctagccaaccttctctcacagtggacggttttacagatccttccaattccgaaaggttctgccttggcctcttgtcaaacgtgaaccggaatgcgactgttgagatgaccagaaggcacattggtcgtggcgttcggctgtactacatcggaggggaagtgttcgctgagtgccttagcgacagtgcgatcttcgtccagagcccaaattgcaaccagcgctatggatggcacccagcgactgtttgtaagattcccccaggctgtaatttgaagatctttaataatcaggagtttgcagccctgttggcccagagtgtgaaccaggggtttgaagcggtgtatcaactgactcgcatgtgcactattcggatgagctttgtgaagggatggggtgccgagtacagaagacagactgttacctctacaccttgttggattgaactccatcttaacggcccactgcaatggcttgataaggtgcttacacagatggggtcaccatcagtgcgctgcgaagagatggaa

SEQ ID NO: 7 - DNA, 1275 bp, Homo sapiens
  source 1..1275: mol_type = genomic DNA
atgtcgtccatcctgcctttcactcccccgatcgtgaagcgcctgctgggctggaagaagggcgagcagaacgggcaggaggagaaatggtgcgagaaggcggtcaagagcctggtcaagaaactcaagaagacggggcagctggacgagctggagaaggccatcaccacgcagaacgtcaacaccaagtgcatcaccatccccaggtccctggatggccggttgcaggtgtcccatcggaaggggctccctcatgtcatctactgccgcctgtggcgatggccagacctgcacagccaccacgagctacgggccatggagctgtgtgagttcgccttcaatatgaagaaggacgaggtctgcgtgaatccctaccactaccagagagtagagacaccagttctacctcctgtgttggtgccacgccacacagagatcccggccgagttccccccactggacgactacagccattccatccccgaaaacactaacttccccgcaggcatcgagccccagagcaatattccagagaccccaccccctggctacctgagtgaagatggagaaaccagtgaccaccagatgaaccacagcatggacgcaggttctccaaacctatccccgaatccgatgtccccagcacataataacttggacctgcagccagttacctactgcgagccggccttctggtgctccatctcctactacgagctgaaccagcgcgtcggggagacattccacgcctcgcagccatccatgactgtggatggcttcaccgacccctccaattcggagcgcttctgcctagggctgctctccaatgtcaacaggaatgcagcagtggagctgacacggagacacatcggaagaggcgtgcggctctactacatcggaggggaggtcttcgcagagtgcctcagtgacagcgctatttttgtccagtctcccaactgtaaccagcgctatggctggcacccggccaccgtctgcaagatcccaccaggatgcaacctgaagatcttcaacaaccaggagttcgctgccctcctggcccagtcggtcaaccagggctttgaggctgtctaccagttgacccgaatgtgcaccatccgcatgagcttcgtcaaaggctggggagcggagtacaggagacagactgtgaccagtaccccctgctggattgagctgcacctgaatgggcctttgcagtggcttgacaaggtcctcacccagatgggctccccaagcatccgctgttccagtgtgtct

SEQ ID NO: 8 - DNA, 1275 bp, synthetic construct
  source 1..1275: mol_type = other DNA
atgtcgtccatcctgcctttcactcccccgatcgtgaagcgcctgctgggctggaagaagggcgagcagaacgggcaggaggagaaatggtgcgagaaggcggtcaagagcctggtcaagaaactcaagaagacggggcagctggacgagctggagaaggccatcaccacgcagaacgtcaacaccaagtgcatcaccatccccaggtccctggatggccggttgcaggtgtcccatcggaaggggctccctcatgtcatctactgccgcctgtggcgatggccagacctgcacagccaccacgagctacgggccatggagctgtgtgagttcgccttcaatatgaagaaggacgaggtctgcgtgaatccctaccactaccagagagtagagacaccagttctacctcctgtgttggtgccacgccacacagagatcccggccgagttccccccactggacgactacagccattccatccccgaaaacactaacttccccgcaggcatcgagccccagagcaatattccagagaccccaccccctggctacctgagtgaagatggagaaaccagtgaccaccagatgaaccacagcatggacgcaggttctccaaacctatccccgaatccgatgtccccagcacataataacttggacctgcagccagttacctactgcgagccggccttctggtgctccatctcctactacgagctgaaccagcgcgtcggggagacattccacgcctcgcagccatccatgactgtggatggcttcaccgacccctccaattcggagcgcttctgcctagggctgctctccaatgtcaacaggaatgcagcagtggagctgacacggagacacatcggaagaggcgtgcggctctactacatcggaggggaggtcttcgcagagtgcctcagtgacagcgctatttttgtccagtctcccaactgtaaccagcgctatggctggcacccggccaccgtctgcaagatcccaccaggatgcaacctgaagatcttcaacaaccaggagttcgctgccctcctggcccagtcggtcaaccagggctttgaggctgtctaccagttgacccgaatgtgcaccatccgcatgagcttcgtcaaaggctggggagcggagtacaggagacagactgtgaccagtaccccctgctggattgagctgcacctgaatgggcctttgcagtggcttgacaaggtcctcacccagatgggctccccaagcatccgctgtgatgacgtggat

SEQ ID NO: 9 - DNA, 1275 bp, synthetic construct
  source 1..1275: mol_type = other DNA
atgtcctccatcttgccttttacaccacctatcgtgaaacgccttctgggttggaaaaaaggcgaacaaaatggccaagaagaaaagtggtgtgagaaggccgtcaaatcattggtgaagaagttgaaaaagacaggacagcttgatgagctggaaaaggccattactacccagaatgttaatactaagtgcatcacaatcccacgctctctggatggaagactgcaagtgagccacaggaaaggtttgccacatgtcatctattgtagactctggagatggccagatttgcatagtcaccatgagctgagagctatggagctttgtgaatttgcattcaatatgaagaaagatgaggtgtgcgttaatccctaccactaccagagagtggaaactcccgttctgcctccagttctggtgccaagacacacagaaattccggcagaatttccccctctggatgattacagtcactcaatccctgagaacacaaacttccctgctgggattgagccccagtctaacatccccgaaacaccaccacctggttatcttagcgaagacggcgaaacttctgaccatcagatgaatcacagcatggatgctggctctcccaatctgagcccaaaccctatgtcaccagcacacaataatctggacttgcagcctgtcacttactgtgaacctgctttttggtgctctattagctattacgagctcaatcagagggtgggagaaacctttcacgctagccaaccctccatgaccgtggatgggttcactgacccaagcaactctgaaagattttgtctgggtctcttgagtaacgtgaacaggaatgcagccgtcgagctcacacgcagacatatcgggagaggagtgcggttgtactatatcggaggcgaagtgttcgccgagtgcctgtcagattctgccatcttcgtccagagcccaaactgtaatcagaggtatggatggcacccagcaacagtgtgtaaaattcctcccggatgcaacctcaagattttcaacaatcaggagtttgccgctcttctcgcacaatccgtgaatcagggtttcgaggccgtgtatcagctgactagaatgtgcacaatccggatgagcttcgttaaaggctggggagccgagtatcgcaggcaaacagttacatcaaccccatgttggatcgagctccacctgaacggaccactgcaatggctagacaaggtcctgacacaaatgggttccccaagtattcggtgtgatgacgtggat

SEQ ID NO: 10 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 11 - protein, 503 aa, synthetic construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARDIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 12 - protein, 470 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 13 - protein, 470 aa, synthetic construct
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARDIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 14 - protein, 33 aa, synthetic construct
MEAAVAAPRPRLLLLVLAAAAAAAAALLPGATA

SEQ ID NO: 15 - protein, 93 aa, synthetic construct
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 16 - protein, 21 aa, synthetic construct
AAVIAGPVCFVCISLMLMVYI

SEQ ID NO: 17 - protein, 356 aa, Homo sapiens
CHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 18 - protein, 356 aa, synthetic construct
CHNRTVIHHRVPNEEDPSLDRPFISEGTTLKDLIYDMTTSGSGSGLPLLVQRTIARDIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 19 - protein, 467 aa, Homo sapiens
MSSILPFTPPVVKRLLGWKKSAGGSGGAGGGEQNGQEEKWCEKAVKSLVKKLKKTGRLDELEKAITTQNCNTKCVTIPSTCSEIWGLSTPNTIDQWDTTGLYSFSEQTRSLDGRLQVSHRKGLPHVIYCRLWRWPDLHSHHELKAIENCEYAFNLKKDEVCVNPYHYQRVETPVLPPVLVPRHTEILTELPPLDDYTHSIPENTNFPAGIEPQSNYIPETPPPGYISEDGETSDQQLNQSMDTGSPAELSPTTLSPVNHSLDLQPVTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCSSMS

SEQ ID NO: 20 - protein, 467 aa, synthetic construct
MSSILPFTPPVVKRLLGWKKSAGGSGGAGGGEQNGQEEKWCEKAVKSLVKKLKKTGRLDELEKAITTQNCNTKCVTIPSTCSEIWGLSTPNTIDQWDTTGLYSFSEQTRSLDGRLQVSHRKGLPHVIYCRLWRWPDLHSHHELKAIENCEYAFNLKKDEVCVNPYHYQRVETPVLPPVLVPRHTEILTELPPLDDYTHSIPENTNFPAGIEPQSNYIPETPPPGYISEDGETSDQQLNQSMDTGSPAELSPTTLSPVNHSLDLQPVTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCEEME

SEQ ID NO: 21 - protein, 425 aa, Homo sapiens
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSIRCSSVS

SEQ ID NO: 22 - protein, 425 aa, synthetic construct
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSIRCDDVD

SEQ ID NO: 23 - protein, 8 aa, synthetic construct
TTSGSGSG

SEQ ID NO: 24 - protein, 4 aa, synthetic construct
SSMS

SEQ ID NO: 25 - protein, 5 aa, synthetic construct
CSSVS

SEQ ID NO: 26 - protein, 4 aa, synthetic construct
GGGS

SEQ ID NO: 27 - protein, 7 aa, synthetic construct
GGGSGGG

SEQ ID NO: 28 - protein, 404 aa, synthetic construct
EMWHEGLEEASRLYFGERNVKGMFEVLEPLHAMMERGPQTLKETSFNQAYGRDLMEAQEWCRKYMKSGNVKDLTQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 29 - protein, 231 aa, synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 30 - protein, 89 aa, synthetic construct
EMWHEGLEEASRLYFGERNVKGMFEVLEPLHAMMERGPQTLKETSFNQAYGRDLMEAQEWCRKYMKSGNVKDLTQAWDLYYHVFRRISK

(19) United States
(12) Patent Application Publication
(10) Pub. No.: US 2025/0127812 A1
(43) Pub. Date: Apr. 24, 2025

(54) COMPOSITIONS AND METHODS FOR ENGINEERING STABLE TREGS

(71) Applicant: GentiBio. Inc., Cambridge, MA (US)

(72) Inventors: Adel Nada, Boston, MA (US); Tom Wickham, Scituate, MA (US)

(73) Assignee: GentiBio. Inc., Cambridge, MA (US)

(21) Appl. No.: 18/682,610

(22) PCT Filed: Aug. 10, 2022
(86) PCT No.: PCT/US2022/074784

§ 371 Date: Feb. 9, 2024

Related U.S. Application Data
(60) Provisional application No. 63/231,679, filed on Aug. 10, 2021.

(51) Int. Cl. A61K 35/17; A61K 40/11; A61K 40/42; C12N 5/0783; C12N 9/22; C12N 15/11; C12N 15/113; C12N 15/86; C12N 15/88

(52) U.S. Cl. CPC A61K 35/17; A61K 40/11; A61K 40/4203; C12N 5/0637; C12N 9/22; C12N 15/111; C12N 15/1138; C12N 15/86; C12N 15/88; C12N 2310/141; C12N 2310/20; C12N 2310/532; C12N 2740/15043; C12N 2750/14143; C12N 2830/002

(57) ABSTRACT

Described herein are compositions and methods for producing regulatory T cells (Tregs) with stable immunosuppressive phenotypes by modifying cells to promote expression of TGFβR1, Smad2, and/or Smad3.

### RELATED APPLICATION

This application claims the benefit under 35 U.S.C. § 119 (c) of U.S. Provisional Application No. 63/231,679, filed Aug. 10, 2021, the contents of which are incorporated by reference herein in their entirety.

### REFERENCE TO AN ELECTRONIC SEQUENCE LISTING

The contents of the electronic Sequence Listing (G097170018WO00-SEQ-NTJ.xml; Size: 44,620 bytes; and Date of Creation: Aug. 9, 2022) are herein incorporated by reference in their entirety.

### BACKGROUND

Cellular therapies using regulatory T cells (Tregs) may be useful to treat numerous types of diseases including autoimmune diseases, allergic diseases, inflammatory diseases, transplant rejection, and grant-versus-host disease.

### SUMMARY

Sorted Tregs isolated from a donor can be used generate a population of Tregs for further manipulation for therapeutic use. Sorting Tregs based on available protein markers yields a heterogenous cell population, which may undermine potency and efficacy of the cell population due to heterogeneity in the strength of the Treg phenotype. Further, Tregs sorted on the basis of protein markers lack stability of suppressive function, as Tregs may convert to an effector T cell (Teff) phenotype when exposed to inflammatory conditions.

Some aspects of the disclosure relate to compositions and methods for improved methods of producing Tregs and Treg populations for therapeutic use. The compositions and methods described herein can stabilize Treg phenotypes and reduce heterogeneity in sorted Treg populations, enhancing their therapeutic potential as cell therapy by increasing their potency and mitigating risks related to instability and potential transdifferentiation to effector T cell phenotypes. The compositions and methods described herein can also be used to induce a stable suppressive phenotype of Treg populations in vivo, e.g., through transduction, transfection, or gene editing of endogenous T cells.

The compositions and methods described herein are based, at least in part, on the recognition that enhancement of TGF-β signaling imparts stability to Treg suppressive phenotype. Some embodiments of the methods described herein involve enhancing constitutive TGF-β signaling by engineering cells to express a constitutively active TGF-β receptor. In some embodiments, cells are engineered to express constitutively active downstream effectors of TGF-β receptors, such as Smad2, Smad3, and/or Smad4. In contrast to sorting-based methods, which are limited by the number of Tregs available in a sample, such methods are scalable, enabling large-scale manufacturing of Tregs for therapeutic use. Ex vivo engineering of Tregs can be achieved using any source of Treg or immune cell made into a Treg cell, such as Tregs isolated from a donor, or Tregs induced from other autologous or allogeneic populations of cells (e.g., bulk peripheral T cells comprising CD3+, CD4+, and/or CD8+ cells).

In some embodiments. Tregs can be produced in vivo using methods described herein. The methods disclosed herein, when used with in vivo targeting of T cells, can be used as an off-the-shelf therapeutic approach to inducing a therapeutically effective and stable Treg population in vivo, e.g., through antigen specific targeting and IL-2 signaling support.

Accordingly, the present disclosure provides, in some aspects, a method of producing a genetically modified cell, the method comprising introducing into the cell a nucleic acid comprising: a heterologous promoter that is operably linked to a sequence encoding TGFβRI, Smad2, and/or Smad3, or one or more functional derivatives of TGFβRI, Smad2, and/or Smad3.

In some embodiments, the promoter is operably linked to a sequence encoding TGFβRI. In some embodiments, the TGFβRI comprises one or more substitutions of amino acids in a GS domain, optionally in the amino acid sequence TTSGSGSG (SEQ ID NO: 23), or at an amino acid corresponding to Thr204, optionally wherein the TGFβRI comprises an aspartate or glutamate at a position corresponding to Thr204, optionally wherein the TGFβRI comprises a T204D substitution.

In some embodiments, the promoter is operably linked to a sequence encoding Smad2 or SMAD3.

In some embodiments, the Smad2 or Smad3 comprises one or more amino acid substitutions of one or more C-terminal serines.

In some embodiments, the Smad2 or Smad3 comprises one or more substitutions in a C-terminal Ser-Ser-X-Ser phosphorylation motif.

In some embodiments, each of the serines in the Ser-Ser-X-Ser phosphorylation motif is substituted with an aspartate or glutamate.

In some embodiments, the Smad2 comprises one or more amino acid substitutions of Ser464, Ser465, and/or Ser467.

In some embodiments, the Smad3 comprises one or more amino acid substitutions of Ser422, Ser423, and/or Ser425.

In some embodiments, the method further comprises introducing into the cell a nuclease or a nucleic acid encoding the nuclease, wherein the nuclease is capable of cleaving a nucleic acid sequence in the endogenous gene.

In some embodiments, the nuclease is a zinc finger nuclease, TALEN, meganuclease, or RNA-guided DNA endonuclease.

In some embodiments, the nuclease is an RNA-guided DNA endonuclease, and wherein the method further comprises introducing into the cell a gRNA comprising a spacer sequence that is complementary to a nucleic acid sequence in the endogenous gene.

In some embodiments, the RNA-guided DNA endonuclease is a Cas endonuclease.

In some embodiments, the Cas endonuclease is a Cas9 endonuclease.

In some aspects, the disclosure relates to a method of producing a genetically modified cell, the method comprising inserting a heterologous promoter into a nucleic acid of a cell genome upstream from a coding sequence of an endogenous TGFβRI, SMAD2, or SMAD3 gene on the nucleic acid, wherein the inserted promoter is operably linked to the endogenous TGFβRI, SMAD2, SMAD3 gene.

In some embodiments, the heterologous promoter is inserted downstream of an endogenous promoter of the endogenous TGFβRI, SMAD2, or SMAD3 gene.

In some embodiments, the heterologous promoter is inserted within an endogenous promoter of the endogenous TGFβRI, SMAD2, or SMAD3 gene, wherein insertion disrupts the endogenous promoter.

In some embodiments, the inserted promoter is operably linked to the endogenous TGFβRI gene, and wherein the nucleic acid comprising the heterologous promoter further comprises:
(i) a 5′ homology arm having homology to a first nucleic acid sequence in the endogenous TGFβRI gene; and
(ii) a 3′ homology arm having homology to a second nucleic acid sequence in the endogenous TGFβRI gene that is downstream from the first nucleic acid sequence in the endogenous TGFβRI,
wherein the heterologous promoter is located between 5′ and 3′ homology arms.

In some embodiments, the method further comprises modifying the endogenous TGFβRI gene to produce a modified TGFβRI gene encoding a modified TGFβRI protein, wherein the modified TGFβRI protein comprises one or more substitutions of amino acids in a GS domain, optionally in the amino acid sequence TTSGSGSG (SEQ ID NO: 23), or at an amino acid corresponding to Thr204, optionally wherein the TGFβRI comprises an aspartate or glutamate at a position corresponding to Thr204, optionally wherein the TGFβRI comprises a T204D substitution,
optionally wherein the modifying comprises introducing into the cell a nuclease or a nucleic acid encoding the nuclease, and a modifying template comprising:
(i) a 5′ homology arm having homology to a sequence in the TGFβRI gene upstream from the nucleotide sequence encoding the amino acids of the GS domain;
(ii) a nucleic acid sequence encoding the substituted amino acids of the GS domain; and
(iii) a 3′ homology arm having homology to a sequence in the TGFβRI gene downstream from the nucleotide sequence encoding the amino acids of the GS domain,
wherein the nuclease is capable of cleaving the TGFβRI gene at a position within TGFβRI gene to promoter insertion of the modifying template to produce the modified TGFβRI gene.

In some embodiments, the inserted promoter is operably linked to the endogenous SMAD2 or SMAD3 gene, and wherein the nucleic acid comprising the heterologous promoter further comprises:
(i) a 5′ homology arm having homology to a first nucleic acid sequence in the endogenous SMAD2 or SMAD3 gene; and
(ii) a 3′ homology arm having homology to a second nucleic acid sequence in the same endogenous gene as the 5′ homology arm,
wherein the heterologous promoter is located between 5′ and 3′ homology arms.

In some embodiments, the method further comprises modifying the endogenous SMAD2 or SMAD3 gene to produce a modified SMAD2 or SMAD3 gene encoding a modified Smad2 or Smad3 protein, wherein the modified Smad2 or Smad3 protein comprises one or more amino acid substitutions of one or more C-terminal serines.

In some embodiments, the modified Smad2 or Smad3 protein comprises one or more substitutions in a C-terminal Ser-Ser-X-Ser phosphorylation motif.

In some embodiments, each of the serines in the Ser-Ser-X-Ser phosphorylation motif is substituted with an aspartate or glutamate.

In some embodiments, the modified Smad2 protein comprises one or more amino acid substitutions of Ser464, Ser465, and/or Ser467.

In some embodiments, the modified Smad3 protein comprises one or more amino acid substitutions of Ser422, Ser424, and/or Ser425.

In some embodiments, the modifying comprises introducing into the cell a nuclease or a nucleic acid encoding the nuclease, and a modifying template comprising:
(i) a 5′ homology arm having homology to a sequence in the SMAD2 or SMAD3 gene upstream from the nucleotide sequence encoding the one or more C-terminal serines;
(ii) a nucleic acid sequence encoding the substituted amino acids of the C-terminal serines; and
(iii) a 3′ homology arm having homology to a sequence in the SMAD2 or SMAD3 gene downstream from the nucleotide sequence encoding the one or more C-terminal serines,
wherein the nuclease is capable of cleaving the SMAD2 or SMAD3 gene at a position within the SMAD2 or SMAD3 gene to promoter insertion of the modifying template to produce the modified SMAD2 or SMAD3 gene.

In some embodiments, the method further comprises introducing into the cell a nuclease or a nucleic acid encoding the nuclease, wherein the nuclease is capable of cleaving a nucleic acid sequence in a targeted locus.

In some embodiments, the nuclease is a zinc finger nuclease, TALEN, meganuclease, or RNA-guided DNA endonuclease.

In some embodiments, the nuclease is an RNA-guided DNA endonuclease, and wherein the method further comprises introducing into the cell a gRNA comprising a spacer sequence that is complementary to a nucleic acid sequence in the targeted locus.

In some embodiments, the RNA-guided DNA endonuclease is a Cas endonuclease.

In some embodiments, the Cas endonuclease is a Cas9 endonuclease.

In some embodiments, the targeted locus is a safe harbor locus.

In some embodiments, the safe harbor locus is a HIPP11 locus, ROSA26 locus, or AAVS1 locus.

In some embodiments, the targeted locus is a TRAC or TRBC locus.

In some embodiments, the promoter is a constitutive promoter.

In some embodiments, the constitutive promoter is an EF-1α, a PGK promoter, or an MND promoter.

In some embodiments, the promoter is an MND promoter.

In some embodiments, the promoter is an inducible promoter.

In some embodiments, the inducible promoter is inducible by a drug or steroid.

In some embodiments, the nucleic acid comprising the heterologous promoter is comprised in a vector.

In some embodiments, the vector is a viral vector.

In some embodiments, the vector is an adenovirus-associated virus (AAV) vector.

In some embodiments, the AAV vector is derived from an AAV of serotype AAV1, AAV2, AAV3, AAV4, AAV5, AAV6, AAV7, AAV8, AAV9, AAV10, or AAV11.

In some embodiments, the viral vector is a lentiviral vector.

In some embodiments, the vector is a plasmid.

In some embodiments, the vector is a bacterial artificial chromosome.

In some embodiments, the vector is a human artificial chromosome.

In some embodiments, the nucleic acid comprising the heterologous promoter is comprised in a lipid nanoparticle.

In some embodiments, the method further comprises reducing expression of Smad7. In some embodiments, reducing expression of Smad7 comprises introducing a SMAD^{null} mutation into at least one SMAD7 allele of the cell genome.

In some embodiments, reducing expression of Smad7 comprises introducing one or more SMAD^{null} mutations into each SMAD7 allele of the cell genome.

In some embodiments, reducing expression of Smad7 comprises removing one or more exons of a SMAD7 allele from the cell genome.

In some embodiments, reducing expression of Smad7 comprises removing one or more exons of each SMAD7 allele from the cell genome.

In some embodiments, reducing expression of Smad7 comprises removing each exon of each SMAD7 allele from the cell genome.

In some embodiments, reducing Smad7 expression comprises introducing into the cell an RNA interference (RNAi) molecule comprising a nucleic acid sequence that is complementary to a coding sequence encoding Smad7.

In some embodiments, the RNAi molecule is an miRNA, siRNA, or shRNA.

In some embodiments, the cell in which Smad7 expression is reduced does not express detectable Smad7.

In some embodiments, the method further comprises reducing expression of IL-6R. In some embodiments, reducing expression of IL-6R comprises introducing an IL6R^{null} mutation into at least one IL6R allele of the cell genome.

In some embodiments, reducing expression of IL-6R comprises introducing one or more IL6R^{null} mutations into each IL6R allele of the cell genome.

In some embodiments, reducing expression of IL-6R comprises removing one or more exons of an IL6R allele from the cell genome.

In some embodiments, reducing expression of IL-6R comprises removing one or more exons of each IL6R allele from the cell genome.

In some embodiments, reducing expression of IL-6R comprises removing each exon of each IL6R allele from the cell genome.

In some embodiments, reducing IL-6R expression comprises introducing into the cell an RNA interference (RNAi) molecule comprising a nucleic acid sequence that is complementary to a coding sequence encoding IL-6R.

In some embodiments, the RNAi molecule is an miRNA, siRNA, or shRNA.

In some embodiments, the cell in which IL-6R expression is reduced does not express detectable IL-6R.

In some embodiments, the method further comprises reducing expression of gp130. In some embodiments, reducing expression of gp130 comprises introducing a GP130^{null} mutation into at least one GP130 allele of the cell genome.

In some embodiments, reducing expression of gp130 comprises introducing one or more GP130″ mutations into each GP130 allele of the cell genome.

In some embodiments, reducing expression of gp130 comprises removing one or more exons of a GP130 allele from the cell genome.

In some embodiments, reducing expression of gp130 comprises removing one or more exons of each GP130 allele from the cell genome.

In some embodiments, reducing expression of gp130 comprises removing each exon of each GP130 allele from the cell genome.

In some embodiments, reducing gp130 expression comprises introducing into the cell an RNA interference (RNAi) molecule comprising a nucleic acid sequence that is complementary to a coding sequence encoding gp130.

In some embodiments, the RNAi molecule is an miRNA, siRNA, or shRNA.

In some embodiments, the cell in which gp130 expression is reduced does not express detectable gp130.

In some aspects, the disclosure relates to a cell made by a method described herein.

In some aspects, the disclosure relates to a genetically modified cell comprising a heterologous promoter operably linked to a cDNA coding sequence encoding TGFβRI, Smad2, or Smad3.

In some embodiments, the heterologous promoter and cDNA coding sequence are located in a safe harbor locus.

In some embodiments, the safe harbor locus is a HIPP11 locus, ROSA26 locus, or AAVS1 locus.

In some embodiments, the heterologous promoter and cDNA coding sequence are located in a TRAC or TRBC locus.

In some embodiments, the heterologous promoter is operably linked to a sequence encoding TGFβRI.

In some embodiments, the TGFβRI comprises one or more substitutions of amino acids in a GS domain, optionally in the amino acid sequence TTSGSGSG (SEQ ID NO: 23), or an amino acid corresponding to Thr204, optionally wherein the TGFβRI comprises an aspartate or glutamate at a position corresponding to Thr204, optionally wherein the TGFβRI comprises a T204D substitution.

In some embodiments, the heterologous promoter is operably linked to a coding sequence encoding Smad2 or Smad3.

In some embodiments, the Smad2 or Smad3 comprises one or more amino acid substitutions of one or more C-terminal serines.

In some embodiments, the Smad2 or Smad3 comprises one or more substitutions in a C-terminal Ser-Ser-X-Ser phosphorylation motif.

In some embodiments, each of the serines in the Ser-Ser-X-Ser phosphorylation motif is substituted with an aspartate or glutamate.

In some embodiments, the Smad2 comprises one or more amino acid substitutions of Ser464, Ser465, and/or Ser467.

In some embodiments, the Smad3 comprises one or more amino acid substitutions of Ser422, Ser423, and/or Ser425.

In some aspects, the disclosure relates to a genetically modified cell comprising a heterologous promoter inserted upstream from a coding sequence of an endogenous TGFβRI, SMAD2, or SMAD3 gene on a nucleic acid of the cell genome, wherein the inserted promoter is operably linked to the endogenous TGFβRI, SMAD2, SMAD3 coding sequence.

In some embodiments, the inserted promoter is operably linked to the endogenous TGFβRI gene.

In some embodiments, the endogenous TGFβRI gene is modified to produce a modified TGFβRI gene encoding a modified TGFβRI, wherein the modified TGFβRI comprises one or more substitutions of amino acids in a GS domain, optionally in the amino acid sequence TTSGSGSG (SEQ ID NO: 23), or at an amino acid corresponding to Thr204, optionally wherein the TGFβRI comprises an aspartate or glutamate at a position corresponding to Thr204, optionally wherein the TGFβRI comprises a T204D substitution.

In some embodiments, the heterologous promoter is operably linked to the endogenous SMAD2 or SMAD3 gene.

In some embodiments, the endogenous SMAD2 or SMAD3 gene is modified to produce a modified SMAD2 or SMAD3 gene encoding a modified Smad2 or Smad3, wherein the modified Smad2 or Smad3 comprises one or more amino acid substitutions of one or more C-terminal serines.

In some embodiments, the modified Smad2 or Smad3 comprises one or more substitutions in a C-terminal Ser-Ser-X-Ser phosphorylation motif.

In some embodiments, each of the serines in the Ser-Ser-X-Ser phosphorylation motif is substituted with an aspartate or glutamate.

In some embodiments, the modified Smad2 comprises one or more amino acid substitutions of Ser464, Ser465, and/or Ser467.

In some embodiments, the modified Smad3 comprises one or more amino acid substitutions of Ser422, Ser423, and/or Ser425.

In some embodiments, the promoter is a constitutive promoter.

In some embodiments, the constitutive promoter is an EF-1a, a PGK promoter, or an MND promoter.

In some embodiments, the promoter is an MND promoter.

In some embodiments, the promoter is an inducible promoter.

In some embodiments, the inducible promoter is inducible by a drug or steroid.

In some embodiments, the cell comprises a SMAD^{null} mutation in at least one SMAD7 allele of the cell genome.

In some embodiments, the cell comprises a SMAD^{null} mutation in each SMAD7 allele of the cell genome.

In some embodiments, the cell comprises a SMAD7 knockout allele.

In some embodiments, the cell is homozygous for a SMAD7 knockout allele.

In some embodiments, the cell does not express detectable SMAD7.

In some embodiments, the cell comprises a IL6R^{null} mutation in at least one IL6R allele of the cell genome.

In some embodiments, the cell comprises a IL6R^{null} mutation in each IL6R allele of the cell genome.

In some embodiments, the cell comprises a IL6R knockout allele.

In some embodiments, the cell is homozygous for a IL6R knockout allele.

In some embodiments, the cell does not express detectable IL-6R.

In some embodiments, the cell comprises a GP130″ mutation in at least one GP130 allele of the cell genome.

In some embodiments, the cell comprises a GP130^{null} mutation in each GP130 allele of the cell genome.

In some embodiments, the cell comprises a GP130 knockout allele.

In some embodiments, the cell is homozygous for a GP130 knockout allele.

In some embodiments, the cell does not express detectable gp130.

In some embodiments, the cell is a stem cell or T cell.

In some embodiments, the cell is a CD3+, CD4+, or CD8+ T cell.

In some embodiments, the cell is a Treg cell.

In some embodiments, the cell is a FoxP3+ Treg cell.

In some embodiments, the cell is CTLA-4+, LAG-3+, CD25+, CD39+, CD27+, CD70+, GITR+, neuropilin-1+, galectin-1+, and/or IL-2Rα+.

In some aspects, the disclosure relates to a pharmaceutical composition comprising a cell described herein.

In some aspects, the disclosure relates to a method comprising administering a cell or pharmaceutical described herein to a subject.

In some embodiments, the subject has or is at risk of developing an inflammatory disease, autoimmune disease, allergic disease, or a condition associated with a solid organ transplant.

In some embodiments, the inflammatory disease is selected from pancreatic islet cell transplantation, asthma, hepatitis, traumatic brain injury, primary sclerosing cholangitis, primary biliary cholangitis, polymyositis, stroke, Still's disease, acute respiratory distress syndrome (ARDS), uveitis, inflammatory bowel disease (IBD), ulcerative colitis, graft-versus-host disease (GvHD), tolerance induction for transplantation, transplant rejection, or sepsis.

In some embodiments, the autoimmune disease is type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, or celiac disease.

In some embodiments, the allergic disease is allergic asthma, steroid-resistant asthma, atopic dermatitis, celiac disease, pollen allergy, food allergy, drug hypersensitivity, or contact dermatitis.

In some embodiments, the condition associated with a solid organ transplant is graft-versus-host disease.

In some embodiments, the subject has or is at risk of developing type 1 diabetes. In some embodiments, the subject has or is at risk of developing inflammatory bowel disease.

In some embodiments, the subject has or is at risk of developing multiple sclerosis. In some embodiments, the subject has or is at risk of developing primary biliary cholangitis.

In some embodiments, the subject has or is at risk of developing acute respiratory distress syndrome.

In some embodiments, the subject has or is at risk of developing stroke.

In some embodiments, the subject has or is at risk of developing graft-versus-host disease.

In some embodiments, the cell is autologous to the subject.

In some embodiments, the cell is an allogeneic cell.

In some aspects, the disclosure relates to a nucleic acid comprising a promoter that is operably linked to a coding sequence encoding TGFβRI, Smad2, and/or Smad3, or one or more functional derivatives of TGFβRI, Smad2, and/or Smad3.

In some embodiments, the coding sequence is a cDNA coding sequence that does not comprise an intron.

In some embodiments, the nucleic acid further comprises:
(i) a 5′ homology arm having homology to a sequence in a targeted locus in a human genome; and
(ii) a 3′ homology arm having homology to a sequence in the same targeted locus as the 5′ homology arm,

In some embodiments, the promoter and coding sequence are located between the 5′ and 3′ homology arms.

In some embodiments, the coding sequence encodes TGFβRI or a functional derivative thereof.

In some embodiments, the TGFβRI comprises one or more substitutions of amino acids in a GS domain, optionally in the amino acid sequence TTSGSGSG (SEQ ID NO: 23), or at an amino acid corresponding to Thr204, optionally wherein the TGFβRI comprises an aspartate or glutamate at a position corresponding to Thr204, optionally wherein the TGFβRI comprises a T204D substitution.

In some embodiments, the coding sequence encodes Smad2, Smad3, or a functional derivative thereof.

In some embodiments, the Smad2 or Smad3 comprises one or more amino acid substitutions of one or more C-terminal serines.

In some embodiments, the Smad2 or Smad3 comprises one or more substitutions in a C-terminal Ser-Ser-X-Ser phosphorylation motif.

In some embodiments, each of the serines in the Ser-Ser-X-Ser phosphorylation motif is substituted with an aspartate or glutamate.

In some embodiments, the Smad2 comprises one or more amino acid substitutions of Ser464, Ser465, and/or Ser467.

In some embodiments, the Smad3 comprises one or more amino acid substitutions of Ser422, Ser423, and/or Ser425.

In some embodiments, the targeted locus is a safe harbor locus.

In some embodiments, the safe harbor locus is a HIPP11 or AAVS1 locus.

In some embodiments, the targeted locus is a TRAC or TRBC locus.

In some embodiments, the promoter is a constitutive promoter.

In some embodiments, the constitutive promoter is an EF-1a, a PGK promoter, or an MND promoter.

In some embodiments, the promoter is an MND promoter.

In some embodiments, the promoter is an inducible promoter.

In some embodiments, the inducible promoter is inducible by a drug or steroid.

In some aspects, the disclosure relates to a vector comprising a nucleic acid described herein.

In some embodiments, the vector is a viral vector.

In some embodiments, the vector is an adenovirus-associated virus (AAV) vector.

In some embodiments, the AAV vector is derived from an AAV of serotype AAV1, AAV2, AAV3, AAV4, AAV5, AAV6, AAV7, AAV8, AAV9, AAV10, or AAV11.

In some embodiments, the viral vector is a lentiviral vector.

In some embodiments, the vector is a plasmid.

In some embodiments, the vector is a bacterial artificial chromosome.

In some embodiments, the vector is a human artificial chromosome.

In some embodiments, the vector further comprises a nucleic acid sequence encoding an RNAi molecule comprising a sequence that is complementary to a sequence within a coding sequence encoding Smad7, IL-6R, or gp130.

In some embodiments, the RNAi molecule is an miRNA, shRNA, or siRNA.

In some aspects, the disclosure relates to a lipid nanoparticle comprising a nucleic acid or vector described herein.

In some embodiments, the lipid nanoparticle further comprises an RNAi molecule comprising a sequence that is complementary to a sequence within a coding sequence encoding Smad7, IL-6R, or gp130.

In some embodiments, the RNAi molecule is an miRNA, shRNA, or siRNA.

In some aspects, the disclosure relates to a system comprising a nucleic acid, vector, and/or lipid nanoparticle described herein, and a nuclease or nucleic acid encoding the nuclease, where the nuclease is capable of cleaving a nucleic acid sequence in the targeted locus.

In some embodiments, the nuclease is a zinc finger DNA endonuclease, TALEN, meganuclease, or RNA-guided DNA endonuclease.

In some embodiments, the nuclease is an RNA-guided DNA endonuclease.

In some embodiments, the RNA-guided DNA endonuclease is a Cas endonuclease.

In some embodiments, the Cas endonuclease is a Cas9 endonuclease.

In some aspects, the disclosure relates to a pharmaceutical composition comprising a nucleic acid, vector, lipid nanoparticle described herein; and a pharmaceutically acceptable excipient.

In some aspects, the disclosure relates to a method comprising administering a nucleic acid, vector, lipid nanoparticle, system, or pharmaceutical composition described herein to a subject.

In some embodiments, the subject has or is at risk of developing an inflammatory disease, autoimmune disease, allergic disease, or a condition associated with a solid organ transplant.

In some embodiments, the inflammatory disease is selected from pancreatic islet cell transplantation, asthma, hepatitis, traumatic brain injury, primary sclerosing cholangitis, primary biliary cholangitis, polymyositis, stroke, Still's disease, acute respiratory distress syndrome (ARDS), uveitis, inflammatory bowel disease (IBD), ulcerative colitis, graft-versus-host disease (GvHD), tolerance induction for transplantation, transplant rejection, or sepsis.

In some embodiments, the autoimmune disease is type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, or celiac disease.

In some embodiments, the allergic disease is allergic asthma, steroid-resistant asthma, atopic dermatitis, celiac disease, pollen allergy, food allergy, drug hypersensitivity, or contact dermatitis.

In some embodiments, the condition associated with a solid organ transplant is graft-versus-host disease.

In some embodiments, the subject has or is at risk of developing type 1 diabetes. In some embodiments, the subject has or is at risk of developing inflammatory bowel disease.

In some embodiments, the subject has or is at risk of developing multiple sclerosis. In some embodiments, the subject has or is at risk of developing primary biliary cholangitis.

In some embodiments, the subject has or is at risk of developing acute respiratory distress syndrome.

In some embodiments, the subject has or is at risk of developing stroke.

In some embodiments, the subject has or is at risk of developing graft-versus-host disease.

### BRIEF DESCRIPTION OF THE DRAWINGS

FIG. 1 shows transduction of cells with viral vectors encoding Smad2mut, Smad3mut, Smad2mut and Smad3mut, or TGFβRI-T204D and TGFβRII. Percentages of transduction are measured by expression of EGFR as represented in dot plots and bar graph.

FIGS. 2A-2B show expression of Smad2, Smad3, TGFβRI, and/or TGFβRII in cells transduced with viral vectors encoding such proteins. FIG. 2A shows transduction of cells with viral vectors encoding Smad2mut and/or Smad3mut. FIG. 2B shows transduction of cells with viral vectors encoding TGFβRI-T204D and TGFβRII. Percentages of transduction efficiency are measured by expression of Smad2mut, Smad3mut, and TGFβRI-T204D as represented in dot plots and bar graphs.

FIG. 3 shows expression of FoxP3, CD25, CD27, CD70, and/or GITR in cells transduced with viral vectors encoding Smad2mut, Smad3mut, and TGFβRI-T204D with TGFβRII. Percentages of Tregs are measured by expression of FoxP3 and CD25 as represented in contour plots and bar graph (top row). Percentages of Tregs expressing CD27, CD70, and GITR as represented in contour plots and histograms (middle and bottom rows).

### DETAILED DESCRIPTION

Aspects of the disclosure relate to methods and compositions for producing engineered Treg cells that have stable suppressive function, e.g., by stabilizing FoxP3 expression. In some embodiments, methods described herein comprise inducing or increasing TGF-β signaling in a cell to stabilize FoxP3 expression. In some embodiments, TGF-β signaling is induced or increased by expressing one or more components of the TGF-β signaling pathway (e.g., by expressing recombinant TGF-β receptor, Smad2 or Smad3) in a target cell (e.g., ex vivo, or in vivo). In some embodiments, TGF-β signaling is induced or increased by inhibiting one or more negative regulators of the TGF-β signaling pathway (e.g., by inhibiting Smad7) in a target cell (e.g., ex vivo, or in vivo).

TGF-β, a potent regulator of cell growth, differentiation, apoptosis, and carcinogenesis (17-20), is under androgenic and other controls. TGF-β signals through a cooperative interaction with two cell surface serine/threonine kinase receptors, TGFβRI and TGFβRII. TGF-β first associates with constitutively active TGFβRII, which then recruits and activates TGFβRI kinase by transphosphorylation at a juxtamembrane glycine-serine repeat. With the help of Smad anchor for receptor activation, phosphorylated TGFβRI is able to activate Smad2 and Smad3, for example, by phosphorylating their carboxyl-terminal serine-serine-Xaa-serine motifs. Active Smad2 and/or Smad3 can form heteromeric complexes with Smad4, and either directly or through interactions with transcription factors and co-regulators bind to Smad-binding elements (SBEs) in TGF-β-regulated genes. Further activation of Smad2 and Smad3 is blocked by Smad7, whose expression is induced upon TGF-β stimulation.

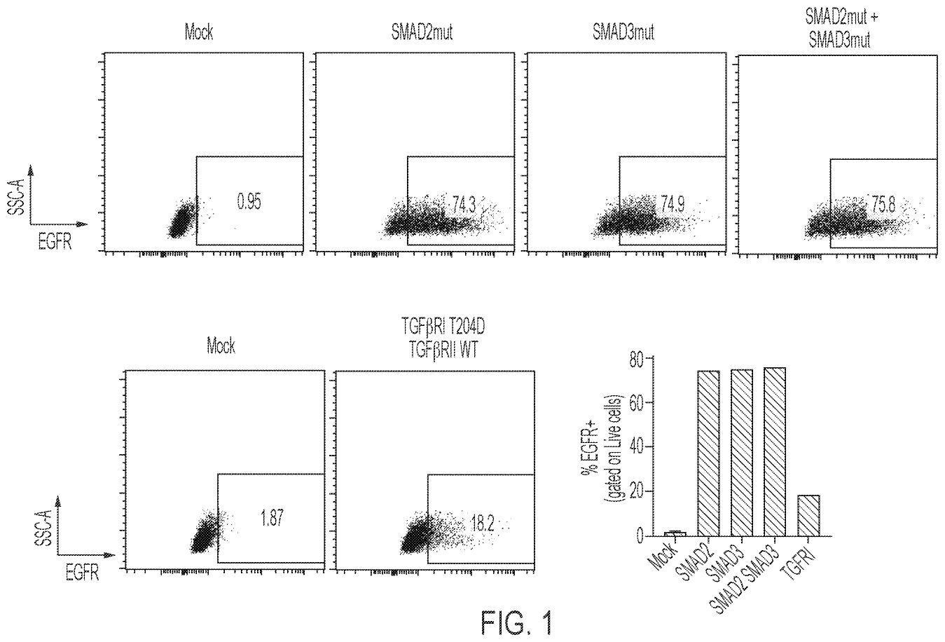

Aspects of the present disclosure relate to engineering FoxP3 regulatory pathways and signals, for example those described in FIG. 1 of Kanamori et al., Trends Immunol 2016 November;37 (11): 803-811. TGF-β signaling-deficient mice exhibited normal tTreg development in the thymus, but peripheral Tregs were significantly reduced in number. A major signaling pathway induced by TGF-β is the phosphorylation and activation of Smad transcription factors. In T cells, two subunits of Smad2 and/or Smad3 are activated by TGF-β, and subsequently form a heterotrimer with Smad4 (e.g., comprising a Smad4 monomer and either two Smad2 monomers, two Smad3 monomers, or a Smad2 and a Smad3 monomer). Smad2 and Smad3 are redundantly essential for FoxP3 induction. T cell-specific Smad2 and Smad3-deficient mice possess normal tTreg cells in the thymus, but total Treg cell number is decreased in the periphery.

Some aspects of the disclosure relate to compositions, cells, nucleic acids, and vectors, and treatment modalities related to genetically modified cells, e.g., regulatory T cells (Tregs), in which signals downstream of TGF-β are stabilized in the cell. The cells described herein are useful, for example, to mitigate and/or prevent certain signs and symptoms of autoimmune, allergic, inflammatory, and other immunopathologic conditions (e.g., symptoms associated with organ transplantation).

The compositions, cells, nucleic acids, and vectors provided herein address the problem of instability in the Treg phenotype. For example, Treg cells may transdifferentiate into a T effector (Teff) cell phenotype in certain conditions (e.g., inflammatory conditions), reducing Treg-mediated immunosuppression of immunopathology (due to reduction in Treg abundance) and exacerbating immunopathology (due to effector function of the transdifferentiated Teff cell). Additionally, signaling activity that occurs downstream of TGF-β may, in combination with signals from IL-6, cause Tregs to transdifferentiate to Teff cells of the T helper 17 (Th17) subset. See, e.g., Kimura and Kishimoto. Eur J Immunol. 2010. 40 (7): 1830-1835. Thus, in some embodiments, the compositions and methods described herein relate to inhibition of IL-6 signaling in cells. For example, genetically modified cells in which a constitutively active TGF-β receptor or adaptor protein (e.g., Smad2) is expressed may also contain IL6R^{null} mutations in one or both genomic IL6R or GP130 alleles, such the cell does not respond to IL-6, Such genetically modified cells maintain a stable Treg phenotype through stabilization of TGF-β signaling, even in the presence of IL-6 that might otherwise convert the Treg into a Th17 cell.

### Methods for Expressing an Exogenous Coding Sequence in a Cell

Some aspects of the disclosure relate to methods of producing a genetically modified cell by introducing into the cell a nucleic acid comprising a heterologous promoter that is operably linked to a coding sequence encoding TGFβRI, Smad2, and/or Smad3, and/or one or more functional derivatives of TGFβRI, Smad2, and/or Smad3. Such a nucleic acid may be integrated into the genome of a cell (e.g., by homologous recombination), such that the encoded gene products (e.g., TGFβRI) are expressed from the genome. Alternatively, the encoded gene products (e.g., TGFβRI) may be expressed episomally.

In some embodiments, the coding sequence encoding TGFβRI, Smad2, and/or Smad3 does not comprise an intron (e.g., the coding sequence is a cDNA sequence). Transcription of mammalian genes generally yields RNA containing multiple exons of a coding sequence that are separated by intervening regions (introns), and processing of this RNA includes RNA splicing to remove introns, yielding an RNA comprising an open reading frame that is capable of being translated by ribosomes and tRNAs to produce the encoded polypeptide. Expression of a gene product from a coding sequence without introns (e.g., a cDNA sequence) abrogates the need for splicing, thereby allowing more rapid and efficient gene expression from an intron-deficient coding sequence relative to a coding sequence that must be spliced between transcription and translation.

In some embodiments, a method comprises contacting a cell with one or more nucleic acids to produce the genetically modified cell. In some embodiments, introducing a nucleic acid, protein, or vector into the cell comprises contacting the cell with the nucleic acid, protein, or vector, respectively. In some embodiments, the cell is in vitro. In some embodiments, the cell is ex vivo. In some embodiments, a cell is isolated from a subject, contacted with one or more nucleic acids, and administered to the same subject. In some embodiments, the cell is ex vivo. In some embodiments, a cell is isolated from a subject, contacted with one or more nucleic acids, and administered to a different subject. In some embodiments, the cell is in vivo.

### TGF-β Receptor I Expression

Some embodiments of the methods described herein comprise introducing a nucleic acid comprising a heterologous promoter operably linked to a nucleic acid sequence encoding TGFβRI, or a functional derivative thereof, into a cell. The functional derivative of TGFβRI may include a protein that has a substantial activity of a wild-type TGFβRI, or increased activity relative to wild-type TGFβRI. One of ordinary skill in the art may use any method known in the art (e.g., phosphorylation assays and/or assaying changes in gene expression following stimulation with TGF-β) to test the functionality or activity of a TGFβRI or derivative thereof. The functional derivative of TGFβRI may also include any TGFβRI or fragment thereof that has conservative substitutions of one or more amino acid residues relative to full-length, wild-type TGFβRI as set forth in SEQ ID NO: 10 (UniProt Accession No. P36897).

In some embodiments, the encoded TGFβRI or functional derivative thereof has about or at least 30%, 40%, 50%, 60%, 70%, 80%, 85%, 90%, 95%, 96%, 97%, 98%, 99%, 100%, or more of the activity exhibited by a wild-type TGFβRI. In some embodiments, the encoded TGFβRI comprises an amino acid sequence with at least 80%, 85%, 90%, 91%, 92%, 93%, 94%, 95%, 96%, 97%, 98%, 99%, or up to 100% sequence identity to the amino acid sequence of wild-type human TGFβRI set forth as SEQ ID NO: 10. In some embodiments, the encoded TGFβRI comprises the wild-type amino acid sequence of SEQ ID NO: 10. In some embodiments, the encoded TGFβRI consists of the wild-type amino acid sequence of SEQ ID NO: 10. In other embodiments, the encoded TGFβRI comprises the amino acid sequence of SEQ ID NO: 11. In some embodiments, the encoded TGFβRI consists of the amino acid sequence of SEQ ID NO: 11.

In some embodiments, the nucleic acid sequence encoding TGFβRI comprises at least 60%, 70%, 75%, 80%, 85%, 90%, 91%, 92%, 93%, 94%, 95%, 96%, 97%, 98%, 99%, or up to 100% sequence identity to the nucleic acid sequence encoding wild-type human TGFβRI set forth as SEQ ID NO: 1. In some embodiments, the nucleic acid sequence encoding TGFβRI comprises the nucleic acid sequence of SEQ ID NO: 1. In some embodiments, the nucleic acid sequence encoding TGFβRI comprises the nucleic acid sequence of SEQ ID NO: 2. In some embodiments, the coding sequence is codon-optimized for expression in a cell. In some embodiments, the coding sequence is codon-optimized for expression in a human cell. In some embodiments, the nucleic acid sequence encoding TGFβRI comprises the nucleic acid sequence of SEQ ID NO: 3.

In some embodiments, the encoded TGFβRI comprises one or more substitutions of amino acids corresponding to a GS domain of wild-type TGFβRI. The “GS domain” of a TGFβRI refers to a glycine-(G) and serine(S)-rich domain in wild-type TGFβRI that precedes the kinase domain. For example, the amino acid sequence set forth by UniProt Accession No. P36897 includes, at amino acids 185-192, the amino acid sequence TTSGSGSG (SEQ ID NO: 23), and each residue of TTSGSGSG (SEQ ID NO: 23) is considered an amino acid corresponding to a GS domain of wild-type TGFβRI. In some embodiments, the TGFβRI comprises a T204D substitution, which renders the kinase domain of TGFβRI constitutively active (Wieser et al., EMBO J. 1995. 14 (10): 2199-2208). In some embodiments, the TGFβRI comprises a T204E substitution, which may also render the kinase domain of TGFβRI constitutively active. In some embodiments, the TGFβRI comprises an extracellular domain, a transmembrane domain, and a cytoplasmic domain. In some embodiments, the extracellular domain comprises at least 90%, at least 95%, at least 97%, at least 98%, at least 99%, or up to 100% sequence identity to the amino acid sequence set forth as SEQ ID NO: 15. In some embodiments, the transmembrane domain comprises at least 90%, at least 95%, at least 97%, at least 98%, at least 99%, or up to 100% sequence identity to the amino acid sequence set forth as SEQ ID NO: 16, In some embodiments, the transmembrane domain comprises at least 90%, at least 95%, at least 97%, at least 98%, at least 99%, or up to 100% sequence identity to the amino acid sequence set forth as SEQ ID NO: 17. In some embodiments, the transmembrane domain comprises at least 90%, at least 95%, at least 97%, at least 98%, at least 99%, or up to 100% sequence identity to the amino acid sequence set forth as SEQ ID NO: 17, and comprises an aspartate or glutamate at a position corresponding to amino acid 56 of SEQ ID NO: 17 (Thr204 in wild-type TGFβRI). In some embodiments, the transmembrane domain comprises at least 90%, at least 95%, at least 97%, at least 98%, at least 99%, or up to 100% sequence identity to the amino acid sequence set forth as SEQ ID NO: 18. In some embodiments, the TGFβRI further comprises a signal peptide. The signal peptide may be any signal peptide known in the art, such as a wild-type TGFβRI signal peptide having an amino acid sequence set forth in SEQ ID NO: 14, or a different signal peptide (e.g., a CD8 signal peptide).

In some embodiments, the encoded TGFβRI protein comprises one or more substitutions in the amino acid sequence corresponding to TTSGSGSG (SEQ ID NO: 23) of wild-type TGFβRI. In some embodiments, 1, 2, 3, 4, 5, 6, 7, or 8 amino acids corresponding to the GS domain are substituted. In some embodiments, 1, 2, or 3 amino acids corresponding to serine residues are substituted. In some embodiments, 1, 2, or 3 amino acids corresponding to glycine residues are substituted. In some embodiments, each serine residue of the GS domain is substituted. In some embodiments, each serine residue of the GS domain is substituted with the same amino acid. In some embodiments, each glycine residue of the GS domain is substituted. In some embodiments, each glycine residue of the GS domain is substituted with the same amino acid. In some embodiments, each amino acid corresponding to TTSGSGSG (SEQ ID NO: 23) is substituted. In some embodiments, each amino acid corresponding to TTSGSGSG (SEQ ID NO: 23) is substituted with the same amino acid. In some embodiments, an amino acid sequence corresponding to TTSGSGSG (SEQ ID NO: 23) is absent from the encoded TGFβRI (e.g., aligning the encoded TGFβRI amino acid sequence to a wild-type TGFβRI sequence shows a gap corresponding to TTSGSGSG (SEQ ID NO: 23)).

### Smad2 Expression

Some embodiments of the methods described herein comprise introducing a nucleic acid comprising a heterologous promoter operably linked to a nucleic acid sequence encoding Smad2, or a functional derivative thereof, into a cell. The functional derivative of Smad2 may include a protein that has a substantial activity of a wild-type Smad2, or increased activity relative to wild-type Smad2. One of ordinary skill in the art may use any method known in the art (e.g., phosphorylation assays and/or assaying changes in gene expression following stimulation with TGF-β) to test the functionality or activity of a Smad2 or derivative thereof. The functional derivative of Smad2 may also include any Smad2 or fragment thereof that has conservative substitutions of one or more amino acid residues relative to full-length, wild-type Smad2 as set forth in SEQ ID NO: 19 (UniProt Accession No. Q15796).

In some embodiments, the encoded Smad2 or functional derivative thereof has about or at least 30%, 40%, 50%, 60%, 70%, 80%, 85%, 90%, 95%, 96%, 97%, 98%, 99%, 100%, or more of the activity exhibited by a wild-type Smad2. In some embodiments, the encoded Smad2 comprises an amino acid sequence with at least 80%, 85%, 90%, 91%, 92%, 93%, 94%, 95%, 96%, 97%, 98%, 99%, or up to 100% sequence identity to the amino acid sequence of wild-type human Smad2 set forth as SEQ ID NO: 19. In some embodiments, the encoded Smad2 comprises the wild-type amino acid sequence of SEQ ID NO: 19. In some embodiments, the encoded Smad2 consists of the wild-type amino acid sequence of SEQ ID NO: 19. In other embodiments, the encoded Smad2 comprises the amino acid sequence of SEQ ID NO: 20. In some embodiments, the encoded Smad2 consists of the amino acid sequence of SEQ ID NO: 20.

In some embodiments, the nucleic acid sequence encoding Smad2 comprises at least 60%, 70%, 75%, 80%, 85%, 90%, 91%, 92%, 93%, 94%, 95%, 96%, 97%, 98%, 99%, or up to 100% sequence identity to the nucleic acid sequence encoding wild-type human Smad2 set forth as SEQ ID NO: 4. In some embodiments, the nucleic acid sequence encoding SMAD2 comprises the nucleic acid sequence of SEQ ID NO: 4. In some embodiments, the nucleic acid sequence encoding Smad2 comprises the nucleic acid sequence of SEQ ID NO: 5. In some embodiments, the coding sequence is codon-optimized for expression in a cell. In some embodiments, the coding sequence is codon-optimized for expression in a human cell. In some embodiments, the nucleic acid sequence encoding Smad2 comprises the nucleic acid sequence of SEQ ID NO: 6.

In some embodiments, the encoded Smad2 comprises one or more substitutions of amino acids corresponding to one or more C-terminal serines of wild-type Smad2. “C-terminal serines of wild-type Smad2” refer to serine residues in an amino acid sequence that correspond (e.g., align) to amino acids 464-467 of wild-type human Smad2. For example, the amino acid sequence set forth by UniProt Accession No. Q15796 includes, at the C-terminus of its amino acid sequence, SSMS (SEQ ID NO: 24), and each serine residue of SEQ ID NO: 24 is considered a C-terminal serine of wild-type SMAD2. This C-terminal motif, containing Ser-Ser-X-Ser in a wild-type Smad2, is known in the art to be a phosphorylation motif, as one or more amino acids (e.g., serines) may become phosphorylated at this motif, and complex with Smad4 when phosphorylated. However, substitution of one or more serines at this motif allows a modified Smad2 to mimic the structure of phosphorylated Smad2, independently of upstream kinase activity that would otherwise modulate Smad2 phosphorylation. Funaba and Mathews, Mol Endocrinol. 2000. 14 (10): 1583-1591. A modified Smad2 that mimics the structure of phosphorylated wild-type Smad2 may thus act constitutively, providing consistent signaling activity in a cell, such as that which would occur in a cell exposed to a consistent amount of TGF-β.

In some embodiments, the encoded Smad2 comprises one or more substitutions in an amino acid sequence corresponding to C-terminal Ser-Ser-X-Ser phosphorylation motif of wild-type Smad2. In some embodiments, one or more amino acids corresponding to Ser464, Ser465, or Ser467 of wild-type Smad2 are substituted in the encoded Smad2. In some embodiments, each of the serines in the Ser-Ser-X-Ser phosphorylation motif of wild-type Smad2 is substituted with an aspartate in the encoded SMAD2 (e.g., the encoded Smad2 comprises the amino acid sequence Asp-Asp-X-Asp at amino acids corresponding to amino acids 464-467 of wild-type Smad2). In some embodiments, each of the serines is substituted with a glutamate. In some embodiments, each of the serines is substituted with a glutamate or aspartate.

### Smad3 Expression

Some embodiments of the methods described herein comprise introducing a nucleic acid comprising a heterologous promoter operably linked to a nucleic acid sequence encoding Smad3, or a functional derivative thereof, into a cell. The functional derivative of Smad3 may include a protein that has a substantial activity of a wild-type Smad3, or increased activity relative to wild-type Smad3. One of ordinary skill in the art may use any method known in the art (e.g., phosphorylation assays and/or assaying changes in gene expression following stimulation with TGF-β) to test the functionality or activity of a Smad3 or derivative thereof. The functional derivative of Smad3 may also include any Smad3 or fragment thereof that has conservative substitutions of one or more amino acid residues relative to full-length, wild-type Smad3 as set forth in SEQ ID NO: 21 (UniProt Accession No. P84022).

In some embodiments, the encoded Smad3 or functional derivative thereof has about or at least 30%, 40%, 50%, 60%, 70%, 80%, 85%, 90%, 95%, 96%, 97%, 98%, 99%, 100%, or more of the activity exhibited by a wild-type Smad3. In some embodiments, the encoded Smad3 comprises an amino acid sequence with at least 80%, 85%, 90%, 91%, 92%, 93%, 94%, 95%, 96%, 97%, 98%, 99%, or up to 100% sequence identity to the amino acid sequence of wild-type human Smad3 set forth as SEQ ID NO: 21. In some embodiments, the encoded Smad3 comprises the wild-type amino acid sequence of SEQ ID NO: 21. In some embodiments, the encoded Smad3 consists of the wild-type amino acid sequence of SEQ ID NO: 21. In other embodiments, the encoded Smad3 comprises the amino acid sequence of SEQ ID NO: 22. In some embodiments, the encoded Smad3 consists of the amino acid sequence of SEQ ID NO: 22.

In some embodiments, the nucleic acid sequence encoding Smad3 comprises at least 60%, 70%, 75%, 80%, 85%, 90%, 91%, 92%, 93%, 94%, 95%, 96%, 97%, 98%, 99%, or up to 100% sequence identity to the nucleic acid sequence encoding wild-type human Smad3 set forth as SEQ ID NO: 7. In some embodiments, the nucleic acid sequence encoding Smad3 comprises the nucleic acid sequence of SEQ ID NO: 7. In some embodiments, the nucleic acid sequence encoding Smad3 comprises the nucleic acid sequence of SEQ ID NO: 8. In some embodiments, the coding sequence is codon-optimized for expression in a cell. In some embodiments, the coding sequence is codon-optimized for expression in a human cell. In some embodiments, the nucleic acid sequence encoding Smad3 comprises the nucleic acid sequence of SEQ ID NO: 9.

In some embodiments, the encoded Smad3 comprises one or more substitutions of terminal serines of wild-type Smad3″ refer to serine residues in an amino acid sequence that correspond (e.g., align) to amino acids 421-425 of wild-type human Smad3. For example, the amino acid sequence set forth by UniProt Accession No. P84022 includes, at the C-terminus of its amino acid sequence, CSSVS (SEQ ID NO: 25), and each serine residue of SEQ ID NO: 25 is considered a C-terminal serine of wild-type Smad3. This C-terminal motif, containing Ser-Ser-X-Ser in a wild-type Smad3, is known in the art to be a phosphorylation motif, as one or more amino acids (e.g., serines) may become phosphorylated at this motif, and complex with SMAD4 when phosphorylated. However, substitution of one or more serines at this motif allows a modified Smad3 to mimic the structure of phosphorylated Smad3, independently of upstream kinase activity that would otherwise modulate Smad3 phosphorylation. Chipuk et al., J Biol Chem. 2002. 277 (2): 1240-1248. A modified Smad3 that mimics the structure of phosphorylated wild-type Smad3 may thus act constitutively, providing consistent signaling activity in a cell, such as that which would occur in a cell exposed to a consistent amount of TGF-β.

In some embodiments, the encoded Smad3 comprises one or more substitutions in an amino acid sequence corresponding to C-terminal Ser-Ser-X-Ser phosphorylation motif of wild-type Smad3. In some embodiments, each of the serines in the Ser-Ser-X-Ser phosphorylation motif of wild-type Smad3 is substituted with an aspartate in the encoded Smad3 (e.g., the Smad3 comprises the amino acid sequence Asp-Asp-X-Asp at amino acids corresponding to amino acids 422-425 of wild-type Smad3). In some embodiments, each of the serines is substituted with a glutamate. In some embodiments, each of the serines is substituted with a glutamate or aspartate. In some embodiments, one or more amino acids corresponding to Ser422, Ser423, or Ser425 of wild-type Smad3 are substituted in the encoded Smad3.

### Targeted Loci

Nucleic acids comprising heterologous promoters operably linked to a coding sequence (e.g., a nucleic acid sequence encoding TGFβRI, Smad2, or Smad3) may be inserted into a targeted locus, such that a population of genetically modified cells contain the inserted sequences at a consistent location of the genome. Such consistency is useful, for example, in screening cells and cell populations by analyzing the targeted locus (e.g., by PCR amplification of genomic DNA using primers flanking the insertion site).

In some embodiments, the nucleic acid comprising a heterologous promoter that is introduced into the cell is inserted at a targeted locus. The targeted locus may correspond to one or more polypeptides encoded by the nucleic acid (e.g., a nucleic acid encoding TGFβRI is inserted at a TGFβRI locus). In such embodiments, the sequence inserted into the targeted locus may replace all or part of the endogenous coding sequence encoding the polypeptide. In some embodiments, one or more mutations (e.g., nonsense mutation) is introduced into the endogenous coding sequence to prevent translation of a full-length polypeptide from the endogenous coding sequence. In some embodiments, all or part of the endogenous coding sequence is removed from the genome by insertion of the heterologous promoter and coding sequence on the inserted nucleic acid.

In some embodiments, the targeted locus is a safe harbor locus. In some embodiments, the safe harbor locus is a HIPP11 locus. In some embodiments, the safe harbor locus is a ROSA26 locus. In some embodiments, the safe harbor locus is an AAVS1 locus. In some embodiments, the targeted locus is a T cell receptor locus. In some embodiments, the T cell receptor locus is a TRAC locus. In some embodiments, the T cell receptor locus is a TRBC locus.

In some embodiments, nucleic acids may be integrated in a non-targeted manner (e.g., by use of a lentiviral vector), such that a population of genetically modified cells contains diverse integration sites.

In some embodiments, the nucleic acid, or vector comprising such a nucleic acid, is not integrated into the genome of the cell. For example, a plasmid or artificial chromosome (e.g., human artificial chromosome) may be introduced into the cell, with the heterologous promoter driving transcription of the operably linked coding sequence from the plasmid or artificial chromosome, without integration of the vector into the chromosome. In some embodiments, the introduced vector or nucleic acid replicates independently of endogenous chromosomes. In some embodiments, more than 1, 2, 3, 4, 5, 6, 7, 8, 9, or 10 copies of the vector are present in a genetically modified cell. In some embodiments, the number of copies of the vector in a cell exceeds the copy number of an individual chromosome in a cell.

### Methods for Stabilizing Expression of an Endogenous Gene in a Cell

Some aspects of the disclosure relate to methods of producing a genetically modified cell by inserting a heterologous promoter into a nucleic acid of a cell genome (e.g., by homologous recombination) upstream from a coding sequence of an endogenous TGFβRI, SMAD2, or SMAD3 gene on the nucleic acid, such that the inserted promoter becomes operably linked to a coding sequence of the endogenous TGFβRI, SMAD2, or SMAD3 gene. In some embodiments, a donor template comprising the heterologous promoter is introduced into the cell, and incorporated into the genome by homologous recombination.

Insertion of a heterologous promoter into the genome allows regulation of a genomic coding sequence (e.g., endogenous TGFβRI coding sequence) in a desired manner, depending on the type and placement of the promoter. For example, placement of a heterologous promoter downstream from an endogenous regulatory element may bypass endogenous regulatory mechanisms (e.g., silencing of the gene in certain conditions, and expression in other conditions), allowing constitutive expression (e.g., by insertion of a constitutive promoter) or tunable expression (e.g., by insertion of an inducible or regulatable promoter and administration of an inducing agent for that promoter).

In some embodiments, the method further comprises modifying an endogenous coding sequence to which the heterologous promoter is operably linked. Such modifications may correct one or more endogenous mutations (e.g., to restore protein function to that of a wild-type protein), remove one more endogenous sequence elements (e.g., introns), or introduce one or more mutations to improve protein function (e.g., inhibit or bypass endogenous regulation).

Insertion of a promoter may modify the coding sequence expressed from the genome. For example, promoter insertion downstream of one or more exons may shorten the sequence of the protein expressed from the endogenous gene. The insertion of a promoter with an additional nucleic acid sequence (e.g., including an in-frame START codon and optionally additional in-frame codons) may incorporate additional amino acids into the N-terminus of the encoded polypeptide. The inclusion of a nucleic acid sequence downstream from the heterologous promoter on the donor template may replace a nucleic acid sequence in the endogenous TGFβRI, SMAD2, or SMAD3 gene, thereby altering the coding sequence and amino acid sequence of the encoded polypeptide. In other embodiments, the heterologous promoter is inserted upstream from the first coding exon of the endogenous coding sequence, and the amino acid sequence of the encoded TGFβRI, Smad2, or Smad3 polypeptide is not altered by insertion of the heterologous promoter.

Modification of an endogenous coding sequence to substitute desired amino acids may be accomplished by any method known in the art. In some embodiments, the donor template comprising the heterologous promoter further comprises a homology arm comprising a modified coding sequence or portion thereof, such that integration of the donor template into the cell genome replaces the codons encoding the substituted amino acids with codons encoding the desired amino acids. In some embodiments, the homology arm comprising the modified coding sequence or portion thereof comprises a homologous nucleic acid sequence downstream from the modified coding sequence or portion thereof, where the homologous nucleic acid sequence is identical to an endogenous sequence downstream from the endogenous coding sequence to be modified, to promote homologous recombination.

In some embodiments, the endogenous coding sequence is modified before insertion of the heterologous promoter. In some embodiments, the endogenous coding sequence is modified after insertion of the heterologous promoter (e.g., by incorporation of a second donor template by a second homologous recombination event).

In some embodiments, a method comprises contacting a cell with one or more nucleic acids to produce the genetically modified cell. In some embodiments, introducing a nucleic acid, protein, or vector into the cell comprises contacting the cell with the nucleic acid, protein, or vector, respectively. In some embodiments, the cell is in vitro. In some embodiments, the cell is ex vivo. In some embodiments, a cell is isolated from a subject, contacted with one or more nucleic acids, and administered to the same subject. In some embodiments, the cell is ex vivo. In some embodiments, a cell is isolated from a subject, contacted with one or more nucleic acids, and administered to a different subject. In some embodiments, the cell is in vivo.

### TGF-β Receptor I Expression

Some embodiments of the methods described herein comprise inserting a heterologous promoter into the genome of a cell upstream from a coding sequence of an endogenous TGFβRI gene, such that the inserted promoter is operably linked to the coding sequence of the endogenous TGFβRI gene. In some embodiments, the method further comprises modifying a coding sequence of the endogenous TGFβRI gene. Such modifications may remove one or more introns and/or mutate one or more exons of the endogenous TGFβRI gene. In some embodiments, the endogenous TGFβRI coding sequence is modified, such that the TGFβRI encoded by the modified TGFβRI coding sequence comprises one or more amino acid substitutions relative to a wild-type TGFβRI (e.g., the wild-type TGFβRI amino acid sequence set forth in SEQ ID NO: 10 (UniProt Accession No. P36897)).

In some embodiments, the modified coding sequence encodes a functional derivative of TGFβRI. The functional derivative of TGFβRI may include a protein that has a substantial activity of a wild-type TGFβRI, or increased activity relative to wild-type TGFβRI. One of ordinary skill in the art may use any method known in the art (e.g., phosphorylation assays and/or assaying changes in gene expression following stimulation with TGF-β) to test the functionality or activity of a TGFβRI or derivative thereof. The functional derivative of TGFβRI may also include any TGFβRI or fragment thereof that has conservative substitutions of one or more amino acid residues relative to full-length, wild-type TGFβRI as set forth in SEQ ID NO: 10 (UniProt Accession No. P36897).

In some embodiments, the encoded TGFβRI or functional derivative thereof has about or at least 30%, 40%, 50%, 60%, 70%, 80%, 85%, 90%, 95%, 96%, 97%, 98%, 99%, 100%, or more of the activity exhibited by a wild-type TGFβRI. In some embodiments, the encoded TGFβRI comprises an amino acid sequence with at least 80%, 85%, 90%, 91%, 92%, 93%, 94%, 95%, 96%, 97%, 98%, 99%, or up to 100% sequence identity to the amino acid sequence of wild-type human TGFβRI set forth as SEQ ID NO: 10. In some embodiments, the encoded TGFβRI comprises the wild-type amino acid sequence of SEQ ID NO: 10. In some embodiments, the encoded TGFβRI consists of the wild-type amino acid sequence of SEQ ID NO: 10. In other embodiments, the encoded TGFβRI comprises the amino acid sequence of SEQ ID NO: 11. In some embodiments, the encoded TGFβRI consists of the amino acid sequence of SEQ ID NO: 11.

In some embodiments, the modified nucleic acid sequence encoding TGFβRI comprises at least 60%, 70%, 75%, 80%, 85%, 90%, 91%, 92%, 93%, 94%, 95%, 96%, 97%, 98%, 99%, or up to 100% sequence identity to the nucleic acid sequence encoding wild-type human TGFβRI set forth as SEQ ID NO: 1. In some embodiments, the modified nucleic acid sequence encoding TGFβRI comprises the nucleic acid sequence of SEQ ID NO: 1. In some embodiments, the modified nucleic acid sequence encoding TGFβRI comprises the nucleic acid sequence of SEQ ID NO: 2. In some embodiments, the coding sequence is codon-optimized for expression in a cell. In some embodiments, the coding sequence is codon-optimized for expression in a human cell. In some embodiments, the modified nucleic acid sequence encoding TGFβRI comprises the nucleic acid sequence of SEQ ID NO: 3.

In some embodiments, the encoded TGFβRI comprises one or more substitutions of amino acids corresponding to a GS domain of wild-type TGFβRI. The “GS domain” of a TGFβRI refers to a glycine-(G) and serine(S)-rich domain in wild-type TGFβRI that precedes the kinase domain. For example, the amino acid sequence set forth by UniProt Accession No. P36897 includes, at amino acids 185-192, the amino acid sequence TTSGSGSG (SEQ ID NO: 23), and each residue of TTSGSGSG (SEQ ID NO: 23) is considered an amino acid corresponding to a GS domain of wild-type TGFβRI. In some embodiments, the TGFβRI comprises a T204D substitution. In some embodiments, the TGFβRI comprises a T204E substitution, which may also render the kinase domain of TGFβRI constitutively active. In some embodiments, the TGFβRI comprises an extracellular domain, a transmembrane domain, and a cytoplasmic domain. In some embodiments, the extracellular domain comprises at least 90%, at least 95%, at least 97%, at least 98%, at least 99%, or up to 100% sequence identity to the amino acid sequence set forth as SEQ ID NO: 15. In some embodiments, the transmembrane domain comprises at least 90%, at least 95%, at least 97%, at least 98%, at least 99%, or up to 100% sequence identity to the amino acid sequence set forth as SEQ ID NO: 16, In some embodiments, the transmembrane domain comprises at least 90%, at least 95%, at least 97%, at least 98%, at least 99%, or up to 100% sequence identity to the amino acid sequence set forth as SEQ ID NO: 17. In some embodiments, the transmembrane domain comprises at least 90%, at least 95%, at least 97%, at least 98%, at least 99%, or up to 100% sequence identity to the amino acid sequence set forth as SEQ ID NO: 17, and comprises an aspartate or glutamate at a position corresponding to amino acid 56 of SEQ ID NO: 17 (Thr204 in wild-type TGFβRI). In some embodiments, the transmembrane domain comprises at least 90%, at least 95%, at least 97%, at least 98%, at least 99%, or up to 100% sequence identity to the amino acid sequence set forth as SEQ ID NO: 18. In some embodiments, the TGFβRI further comprises a signal peptide. The signal peptide may be any signal peptide known in the art, such as a wild-type TGFβRI signal peptide having an amino acid sequence set forth in SEQ ID NO: 14, or a different signal peptide (e.g., a CD8 signal peptide).

In some embodiments, the encoded TGFβRI comprises one or more substitutions in the amino acid sequence corresponding to TTSGSGSG (SEQ ID NO: 23) of wild-type TGFβRI. In some embodiments, 1, 2, 3, 4, 5, 6, 7, or 8 amino acids corresponding to the GS domain are substituted. In some embodiments, 1, 2, or 3 amino acids corresponding to serine residues are substituted. In some embodiments, 1, 2, or 3 amino acids corresponding to glycine residues are substituted. In some embodiments, each serine residue of the GS domain is substituted. In some embodiments, each serine residue of the GS domain is substituted with the same amino acid. In some embodiments, each glycine residue of the GS domain is substituted. In some embodiments, each glycine residue of the GS domain is substituted with the same amino acid. In some embodiments, each amino acid corresponding to TTSGSGSG (SEQ ID NO: 23) is substituted. In some embodiments, each amino acid corresponding to TTSGSGSG (SEQ ID NO: 23) is substituted with the same amino acid. In some embodiments, an amino acid sequence corresponding to TTSGSGSG (SEQ ID NO: 23) is absent from the encoded TGFβRI (e.g., aligning the encoded TGFβRI amino acid sequence to a wild-type TGFβRI sequence shows a gap corresponding to TTSGSGSG (SEQ ID NO: 23)).

### Smad2 Expression

Some embodiments of the methods described herein comprise inserting a heterologous promoter into the genome of a cell upstream from a coding sequence of an endogenous SMAD2 gene, such that the inserted promoter is operably linked to the coding sequence of the endogenous SMAD2 gene. In some embodiments, the method further comprises modifying a coding sequence of the endogenous SMAD2 gene. Such modifications may remove one or more introns and/or mutate one or more exons of the endogenous SMAD2 gene. In some embodiments, the endogenous SMAD2 coding sequence is modified, such that the Smad2 encoded by the modified SMAD2 coding sequence comprises one or more amino acid substitutions relative to a wild-type Smad2 (e.g., the wild-type Smad2 amino acid sequence set forth in SEQ ID NO: 19 (UniProt Accession No. Q15796)).

In some embodiments, the modified coding sequence encodes a functional derivative of Smad2. The functional derivative of Smad2 may include a protein that has a substantial activity of a wild-type Smad2, or increased activity relative to wild-type SMAD2. One of ordinary skill in the art may use any method known in the art (e.g., phosphorylation assays and/or assaying changes in gene expression following stimulation with TGF-β) to test the functionality or activity of a Smad2 or derivative thereof. The functional derivative of Smad2 may also include any Smad2 or fragment thereof that has conservative substitutions of one or more amino acid residues relative to full-length, wild-type SMAD2 as set forth in SEQ ID NO: 19 (UniProt Accession No. Q15796).

In some embodiments, the encoded Smad2 or functional derivative thereof has about or at least 30%, 40%, 50%, 60%, 70%, 80%, 85%, 90%, 95%, 96%, 97%, 98%, 99%, 100%, or more of the activity exhibited by a wild-type Smad2. In some embodiments, the encoded Smad2 comprises an amino acid sequence with at least 80%, 85%, 90%, 91%, 92%, 93%, 94%, 95%, 96%, 97%, 98%, 99%, or up to 100% sequence identity to the amino acid sequence of wild-type human Smad2 set forth as SEQ ID NO: 19. In some embodiments, the encoded Smad2 comprises the wild-type amino acid sequence of SEQ ID NO: 19. In some embodiments, the encoded Smad2 consists of the wild-type amino acid sequence of SEQ ID NO: 19. In other embodiments, the encoded Smad2 comprises the amino acid sequence of SEQ ID NO: 20. In some embodiments, the encoded Smad2 comprises the amino acid sequence of SEQ ID NO: 20.

In some embodiments, the nucleic acid sequence encoding Smad2 comprises at least 60%, 70%, 75%, 80%, 85%, 90%, 91%, 92%, 93%, 94%, 95%, 96%, 97%, 98%, 99%, or up to 100% sequence identity to the nucleic acid sequence encoding wild-type human Smad2 set forth as SEQ ID NO: 4. In some embodiments, the nucleic acid sequence encoding Smad2 comprises the nucleic acid sequence of SEQ ID NO: 4. In some embodiments, the nucleic acid sequence encoding Smad2 consists of the nucleic acid sequence of SEQ ID NO: 5. In some embodiments, the coding sequence is codon-optimized for expression in a cell. In some embodiments, the coding sequence is codon-optimized for expression in a human cell. In some embodiments, the nucleic acid sequence encoding SMAD2 comprises the nucleic acid sequence of SEQ ID NO: 6.

In some embodiments, the encoded Smad2 comprises one or more substitutions of amino acids corresponding to one or more C-terminal serines of wild-type Smad2. “C-terminal serines of wild-type Smad2” refer to serine residues in an amino acid sequence that correspond (e.g., align) to amino acids 464-467 of wild-type human Smad2. For example, the amino acid sequence set forth by UniProt Accession No. Q15796 includes, at the C-terminus of its amino acid sequence, SSMS (SEQ ID NO: 24), and each serine residue of SEQ ID NO: 24 is considered a C-terminal serine of wild-type Smad2.

In some embodiments, the encoded Smad2 comprises one or more substitutions in an amino acid sequence corresponding to C-terminal Ser-Ser-X-Ser phosphorylation motif of wild-type Smad2. In some embodiments, one or more amino acids corresponding to Ser464, Ser465, or Ser467 of wild-type Smad2 are substituted in the encoded Smad2. In some embodiments, each of the serines in the Ser-Ser-X-Ser phosphorylation motif of wild-type

Smad2 is substituted with an aspartate in the encoded Smad2 (e.g., the encoded Smad2 comprises the amino acid sequence Asp-Asp-X-Asp at amino acids corresponding to amino acids 464-467 of wild-type Smad2). In some embodiments, each of the serines is substituted with a glutamate. In some embodiments, each of the serines is substituted with a glutamate or aspartate.

### Smad3 Expression

Some embodiments of the methods described herein comprise inserting a heterologous promoter into the genome of a cell upstream from a coding sequence of an endogenous SMAD3 gene, such that the inserted promoter is operably linked to the coding sequence of the endogenous SMAD3 gene. In some embodiments, the method further comprises modifying a coding sequence of the endogenous SMAD3 gene. Such modifications may remove one or more introns and/or mutate one or more exons of the endogenous SMAD3 gene. In some embodiments, the endogenous SMAD3 coding sequence is modified, such that the Smad3 encoded by the modified SMAD3 coding sequence comprises one or more amino acid substitutions relative to a wild-type Smad3 (e.g., the wild-type Smad3 amino acid sequence set forth in SEQ ID NO: 21 (UniProt Accession No. P84022)).

In some embodiments, the modified coding sequence encodes a functional derivative of Smad3. The functional derivative of Smad3 may include a protein that has a substantial activity of a wild-type Smad3, or increased activity relative to wild-type Smad3. One of ordinary skill in the art may use any method known in the art (e.g., phosphorylation assays and/or assaying changes in gene expression following stimulation with TGF-β) to test the functionality or activity of a Smad3 or derivative thereof. The functional derivative of Smad3 may also include any Smad3 or fragment thereof that has conservative substitutions of one or more amino acid residues relative to full-length, wild-type SMAD3 as set forth in SEQ ID NO: 21 (UniProt Accession No. P84022).

In some embodiments, the encoded Smad3 or functional derivative thereof has about or at least 30%, 40%, 50%, 60%, 70%, 80%, 85%, 90%, 95%, 96%, 97%, 98%, 99%, 100%, or more of the activity exhibited by a wild-type Smad3. In some embodiments, the encoded

Smad3 comprises an amino acid sequence with at least 80%, 85%, 90%, 91%, 92%, 93%, 94%, 95%, 96%, 97%, 98%, 99%, or up to 100% sequence identity to the amino acid sequence of wild-type human Smad3 set forth as SEQ ID NO: 21. In some embodiments, the encoded Smad3 comprises the wild-type amino acid sequence of SEQ ID NO: 21. In some embodiments, the encoded Smad3 consists of the wild-type amino acid sequence of SEQ ID NO: 21. In other embodiments, the encoded Smad3 comprises the amino acid sequence of SEQ ID NO: 22. In some embodiments, the encoded Smad3 consists of the amino acid sequence of SEQ ID NO: 22.

In some embodiments, the modified nucleic acid sequence encoding Smad3 comprises at least 60%, 70%, 75%, 80%, 85%, 90%, 91%, 92%, 93%, 94%, 95%, 96%, 97%, 98%, 99%, or up to 100% sequence identity to the nucleic acid sequence encoding wild-type human Smad3 set forth as SEQ ID NO: 7. In some embodiments, the modified nucleic acid sequence encoding Smad3 comprises the nucleic acid sequence of SEQ ID NO: 7. In some embodiments, the modified nucleic acid sequence encoding Smad3 comprises the nucleic acid sequence of SEQ ID NO: 8. In some embodiments, the coding sequence is codon-optimized for expression in a cell. In some embodiments, the coding sequence is codon-optimized for expression in a human cell. In some embodiments, the modified nucleic acid sequence encoding Smad3 comprises the nucleic acid sequence of SEQ ID NO: 9.

In some embodiments, the encoded Smad3 comprises one or more substitutions of terminal serines of wild-type Smad3″ refer to serine residues in an amino acid sequence that correspond (e.g., align) to amino acids 421-425 of wild-type human Smad3. For example, the amino acid sequence set forth by UniProt Accession No. P84022 includes, at the C-terminus of its amino acid sequence, CSSVS (SEQ ID NO: 25), and each serine residue of SEQ ID NO: 25 is considered a C-terminal serine of wild-type Smad3.

In some embodiments, the encoded Smad3 comprises one or more substitutions in an amino acid sequence corresponding to C-terminal Ser-Ser-X-Ser phosphorylation motif of wild-type Smad3. In some embodiments, one or more amino acids corresponding to Ser422, Ser423, or Ser425 of wild-type Smad3 are substituted in the encoded Smad3. In some embodiments, each of the serines in the Ser-Ser-X-Ser phosphorylation motif of wild-type Smad3 is substituted with an aspartate in the encoded SMAD3 (e.g., the Smad3 comprises the amino acid sequence Asp-Asp-X-Asp at amino acids corresponding to amino acids 422-425 of wild-type Smad3). In some embodiments, each of the serines is substituted with a glutamate. In some embodiments, each of the serines is substituted with a glutamate or aspartate.

Methods for Inhibiting Smad7 and/or IL-6 Signaling

Some embodiments of the methods described herein comprise inhibiting one or more signaling pathways in a cell. In some embodiments, a method comprises introducing an inhibitory nucleic acid into a cell to reduce or inhibit expression of Smad7 and/or receptors for IL-6 (e.g., IL-6R and/or gp130). The inhibitory nucleic acid may be, for instance, an siRNA or an antisense molecule that inhibits expression of a protein. The inhibitory nucleic acids may be designed using routine methods in the art.

An inhibitory nucleic acid typically causes specific gene knockdown, while avoiding off-target effects. Various strategies for gene knockdown known in the art can be used to inhibit gene expression. For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, and other small interfering nucleic acid-based molecules known in the art. In some embodiments, vector-based RNAi modalities (e.g., shRNA expression constructs) are used to reduce expression of a gene in a cell. In some embodiments, a vector (e.g., a plasmid or viral vector known in the art or described herein) that expresses an RNAi molecule (e.g., siRNA) is used to regulate gene expression. Expression of the RNAi molecule may be driven by the heterologous promoter operably linked to a sequence encoding TGFβRI, SMAD2, or SMAD3. In some embodiments, a separate promoter may drive expression of the RNAi molecule from the vector. This separate promoter may comprise the same nucleic acid sequence of the heterologous promoter driving TGFβRI, SMAD2, or SMAD3 expression (e.g., the separate promoter is a copy of the heterologous promoter), or comprise a different nucleic acid sequence.

In some cases, the vector is packaged in a virus capable of infecting the cell or subject (e.g., the vector is a viral vector). Exemplary viruses include adenovirus, retrovirus, lentivirus, adeno-associated virus, and others that are known in the art and disclosed herein.

A broad range of RNAi-based modalities may be employed to inhibit expression of a gene in a cell, such as siRNA-based oligonucleotides and/or altered siRNA-based oligonucleotides. Altered siRNA-based oligonucleotides are those modified to alter potency, target affinity, safety profile and/or stability, for example, to render them resistant or partially resistant to intracellular degradation. Modifications, such as phosphorothioates, for example, can be made to nucleic acids or oligonucleotides to increase resistance to nuclease degradation, binding affinity, and/or uptake. In addition, hydrophobization and bioconjugation enhances siRNA delivery and targeting (De Paula et al., RNA. 13 (4): 431-56, 2007) and siRNAs with ribo-difluorotoluyl nucleotides maintain gene silencing activity (Xia et al., ASC Chem. Biol. 1 (3): 176-83, (2006)). siRNAs with amide-linked oligoribonucleosides have been generated that are more resistant to S1 nuclease degradation than unmodified siRNAs (Iwase R et al. 2006 Nucleic Acids Symp Ser 50:175-176). In addition, modification of siRNAs at the 2′-sugar position and phosphodiester linkage confers improved serum stability without loss of efficacy (Choung et al., Biochem. Biophys. Res. Commun. 342 (3): 919-26, 2006).

Other molecules that can be used to inhibit expression of a gene include antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67 (5): 869-76, 1993; Lange et al., Leukemia. 6 (11): 1786-94, 1993; Valera et al., J. Biol. Chem. 269 (46): 28543-6, 1994; Dosaka-Akita et al., Am. J. Clin. Pathol. 102 (5): 660-4, 1994; Feng et al., Cancer Res. 55 (10): 2024-8, 1995; Quattrone et al., Cancer Res. 55 (1): 90-5, 1995; Lewin et al., Nat Med. 4 (8): 967-71, 1998). Ribozymes have also been proposed as a means of both inhibiting gene expression of a mutant gene and of correcting the mutant by targeted trans-splicing (Sullenger and Cech Nature 371 (6498): 619-22, 1994; Jones et al., Nat. Med. 2 (6): 643-8, 1996).

Triple helix approaches have also been investigated for sequence-specific gene suppression. Triple helix forming oligonucleotides have been found in some cases to bind in a sequence-specific manner (Postel et al., Proc. Natl. Acad. Sci. U.S.A. 88 (18): 8227-31, 1991; Duval-Valentin et al., Proc. Natl. Acad. Sci. U.S.A. 89 (2): 504-8, 1992; Hardenbol and Van Dyke Proc. Natl. Acad. Sci. U.S.A. 93 (7): 2811-6, 1996; Porumb et al., Cancer Res. 56 (3): 515-22, 1996). Similarly, peptide nucleic acids have been shown to inhibit gene expression (Hanvey et al., Antisense Res. Dev. 1 (4): 307-17, 1991; Knudsen and Nielson

Nucleic Acids Res. 24 (3): 494-500, 1996; Taylor et al., Arch. Surg. 132 (11): 1177-83, 1997). Minor-groove binding polyamides can bind in a sequence-specific manner to DNA targets and hence may represent useful small molecules for suppression at the DNA level (Trauger et al., Chem. Biol. 3 (5): 369-77, 1996). In addition, suppression has been obtained by interference at the protein level using dominant negative mutant peptides and antibodies (Herskowitz Nature 329 (6136): 219-22, 1987; Rimsky et al., Nature 341 (6241): 453-6, 1989; Wright et al., Proc. Natl. Acad. Sci. U.S.A. 86 (9): 3199-203, 1989). The diverse array of suppression strategies that can be employed includes the use of DNA and/or RNA aptamers that can be selected to target a protein of interest.

Other inhibitor molecules that can be used include antisense nucleic acids (single or double stranded). Antisense nucleic acids include modified or unmodified RNA, DNA, or mixed polymer nucleic acids, and primarily function by specifically binding to matching sequences resulting in modulation of peptide synthesis (Wu-Pong, November 1994, BioPharm, 20-33). Antisense nucleic acid binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

As used herein, the term “antisense nucleic acid” describes a nucleic acid that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

An inhibitory nucleic acid useful in the invention will generally be designed to have partial or complete complementarity with one or more target genes. The target gene may be a gene derived from the cell, an endogenous gene, a transgene, or a gene of a pathogen which is present in the cell after infection thereof. Depending on the particular target gene, the nature of the inhibitory nucleic acid and the level of expression of inhibitory nucleic acid (e.g. depending on copy number, promoter strength) the procedure may provide partial or complete loss of function for the target gene. Quantitation of gene expression in a cell may show similar amounts of inhibition at the level of accumulation of target mRNA or translation of target protein.

“Inhibition of gene expression” refers to the absence or observable decrease in the level of protein and/or mRNA product from a target gene. “Specificity” refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one of ordinary skill to determine a degree of inhibition which is greater than 10%, 33%, 50%, 90%, 95% or 99% as compared to a cell in which gene expression is not reduced by a method described herein. As an example, the efficiency of inhibition may be determined by assessing the amount of gene product in the cell: mRNA may be detected with a hybridization probe having a nucleotide sequence outside the region used for the inhibitory nucleic acid, or translated polypeptide may be detected with an antibody raised against the polypeptide sequence of that region.

In some embodiments, reduction in expression of a target gene (e.g., SMAD7, IL6R, GP130) reduces the amount of the encoded protein (e.g., Smad7, IL-6R. gp130) in the cell below a limit of detection, such that the cell does not express a detectable amount of the encoded protein. Non-limiting examples of such methods include flow cytometry, western blotting, immunoprecipitation, and fluorescent microscopy.

In some embodiments, reduction in target gene expression reduces the amount of mRNA encoding the undesired protein in a cell below a limit of detection, such that the cell does not express a detectable amount of mRNA encoding the undesired protein. In some embodiments, the cell does not transcribe detectable mRNA encoding the undesired protein. Detection of mRNA encoding a given protein, and consequently a determination of whether a cell expresses detectable mRNA encoding the protein, may be accomplished by any method known in the art. Non-limiting examples of such methods include qRT-PCR, RNAseq (e.g., single-cell RNAseq), fluorescent in situ hybridization, and probe-based microscopy.

In some embodiments, a method described herein reduces expression of Smad7-encoding mRNA by 50% or more, 60% or more, 70% or more, 80% or more, 85% or more, 90% or more, 95% or more, 96% or more, 97% or more, 98% or more, 99% or more, or up to 100%, relative to a cell in which SMAD7 expression is not reduced by the described method. In some embodiments, a method described herein reduces expression of IL-6R-encoding mRNA by 50% or more, 60% or more, 70% or more, 80% or more, 85% or more, 90% or more, 95% or more, 96% or more, 97% or more, 98% or more, 99% or more, or up to 100%, relative to a cell in which IL6R expression is not reduced by the described method. In some embodiments, a method described herein reduces expression of gp130-encoding mRNA by 50% or more, 60% or more, 70% or more, 80% or more, 85% or more, 90% or more, 95% or more, 96% or more, 97% or more, 98% or more, 99% or more, or up to 100%, relative to a cell in which GP 130 expression is not reduced by the described method.

In some embodiments, a method described herein reduces the abundance of Smad7 protein in a cell by 50% or more, 60% or more, 70% or more, 80% or more, 85% or more, 90% or more, 95% or more, 96% or more, 97% or more, 98% or more, 99% or more, or up to 100%, relative to a cell in which Smad7 expression is not reduced by the described method. In some embodiments, a method described herein reduces the abundance of IL-6R protein in a cell by 50% or more, 60% or more, 70% or more, 80% or more, 85% or more, 90% or more, 95% or more, 96% or more, 97% or more, 98% or more, 99% or more, or up to 100%, relative to a cell in which IL-6R expression is not reduced by the described method. In some embodiments, a method described herein reduces the abundance of gp130 protein in a cell by 50% or more, 60% or more, 70% or more, 80% or more, 85% or more, 90% or more, 95% or more, 96% or more, 97% or more, 98% or more, 99% or more, or up to 100%, relative to a cell in which gp130 expression is not reduced by the described method.

### SMAD7 Inhibition

In some embodiments, a method comprises reducing or inhibiting expression of SMAD7 in the cell. Reducing SMAD7 expression may be achieved through any method known in the art, such as by use of an inhibitory nucleic acid as described herein. Additionally or alternatively, SMAD7 expression may be reduced by introducing one or more SMAD^{null} mutations into a SMAD7 allele of a cell's genome. In some embodiments, SMAD^{null} mutations are introduced into both SMAD7 alleles of the cell genome. The SMAD^{null} mutations introduced into different alleles of the genome may be the same mutation (e.g., nonsense mutations may be introduced at the same position on both chromosomes) or different mutations (e.g., different nonsense mutations). Non-limiting examples of SMAD^{null} mutations include nonsense mutations (e.g., premature STOP codons) that prevent translation of functional SMAD7, missense mutations (e.g., substitutions) that reduce or abrogate SMAD7 function, and frameshift mutations that prevent translation of a functional SMAD7 polypeptide (e.g., by shortening the encoded amino acid sequence, or by adding, removing, or substituting one or more amino acid residues in a manner that reduces or abrogates SMAD7 function). Nonsense, missense, and frameshift mutations are known in the art, as are methods of determining whether a given mutation impacts SMAD7 function. See, e.g., Zhang et al., Mol Cell Biol. 2007. 27 (12): 4488-4499.

In some embodiments, a method comprises modifying an endogenous SMAD7 promoter or portion thereof, such that less Smad7-encoding RNA is transcribed in cells containing the modified SMAD7 promoter. In some embodiments, the endogenous SMAD7 promoter is modified at both SMAD7 alleles in the cell genome. In some embodiments, both SMAD7 allele promoters are modified in the same manner (e.g., to contain the same modified promoter sequence). In other embodiments, both promoters are modified to contain different sequences, each of which differs from the endogenous promoter sequence.

In some embodiments, a method comprises excising the endogenous SMAD7 promoter from one or both SMAD7 alleles of a genome. For example, a donor template comprising homology arms with homology to sequences upstream and downstream from the endogenous promoter, but lacking the promoter sequence, may be introduced into a cell, to promote replacement of the endogenous promoter with the donor template by homologous recombination, such that modified chromosome does not comprise an endogenous SMAD7 promoter. In some embodiments, the SMAD7 promoter is excised from both SMAD7 alleles. In some embodiments, a nuclease known in the art or described herein is used to cleave one or both chromosomes to promote modification, replacement, or excision of the endogenous SMAD7 promoter by homology-directed repair. In some embodiments, a nuclease known in the art or described herein is used to cleave one or both chromosomes to promote introduction of a SMAD^{null} mutation into the cell genome. In some embodiments, an RNA-guided nuclease is used.

### IL-6 Signaling Inhibition

In some embodiments, a method comprises inhibiting the ability of a cell to respond to IL-6, For example, reducing cell surface expression of receptors of the IL-6 signaling pathway, such as IL-6R and/or gp130, inhibits the occurrence of signal transduction associated with IL-6 (e.g., recruitment of JAK/STAT proteins to IL-6R and gp130) that may otherwise occur in the cell when IL-6 is present. Reduced expression of IL-6R and/or gp130 therefore inhibits undesired effects of IL-6 signaling in cells, such as transdifferentiation of Tregs into Th17 effector cells. In some embodiments, a method comprises reducing or inhibiting expression of IL6R in the cell. Reducing IL6R expression may be achieved through any method known in the art, such as by use of an inhibitory nucleic acid as described herein. Additionally or alternatively, IL6R expression may be reduced by introducing one or more IL6R^{null} mutations into a IL6R allele of a cell's genome. In some embodiments, IL6R^{null} mutations are introduced into both IL6R alleles of the cell genome. The IL6R^{null} mutations introduced into different alleles of the genome may be the same mutation (e.g., nonsense mutations may be introduced at the same position on both chromosomes) or different mutations (e.g., different nonsense mutations). Non-limiting examples of IL6R^{null} mutations include nonsense mutations (e.g., premature STOP codons) that prevent translation of functional IL-6R, missense mutations (e.g., substitutions) that reduce or abrogate IL-6R function, and frameshift mutations that prevent translation of a functional IL-6R polypeptide (e.g., by shortening the encoded amino acid sequence, or by adding, removing, or substituting one or more amino acid residues in a manner that reduces or abrogates IL-6R function). Nonsense, missense, and frameshift mutations are known in the art, as are methods of determining whether a given mutation impacts IL-6R function. See, e.g., Spencer et al., J Exp Med. 2019. 216 (9): 1986-1998.

In some embodiments, a method comprises modifying an endogenous IL6R promoter or portion thereof, such that less IL-6R-encoding RNA is transcribed in cells containing the modified IL6R promoter. In some embodiments, the endogenous IL6R promoter is modified at both IL6R alleles in the cell genome. In some embodiments, both IL6R allele promoters are modified in the same manner (e.g., to contain the same modified promoter sequence). In other embodiments, both promoters are modified to contain different sequences, each of which differs from the endogenous promoter sequence.

In some embodiments, a method comprises excising the endogenous IL6R promoter from one or both IL6R alleles of a genome. For example, a donor template comprising homology arms with homology to sequences upstream and downstream from the endogenous promoter, but lacking the promoter sequence, may be introduced into a cell, to promote replacement of the endogenous promoter with the donor template by homologous recombination, such that modified chromosome does not comprise an endogenous IL6R promoter. In some embodiments, the IL6R promoter is excised from both IL6R alleles.

In some embodiments, a nuclease known in the art or described herein is used to cleave one or both chromosomes to promote modification, replacement, or excision of the endogenous IL6R promoter by homology-directed repair. In some embodiments, a nuclease known in the art or described herein is used to cleave one or both chromosomes to promote introduction of a IL6R^{null} mutation into the cell genome. In some embodiments, an RNA-guided nuclease is used.

In some embodiments, a method comprises, or further comprises, reducing or inhibiting expression of GP 130 in the cell. Reducing GP130 expression may be achieved through any method known in the art, such as by use of an inhibitory nucleic acid as described herein. Additionally or alternatively, GP 130 expression may be reduced by introducing one or more GP130^{null} mutations into a GP130 allele of a cell's genome. In some embodiments, GP130^{null} mutations are introduced into both GP130 alleles of the cell genome. The GP130^{null} mutations introduced into different alleles of the genome may be the same mutation (e.g., nonsense mutations may be introduced at the same position on both chromosomes) or different mutations (e.g., different nonsense mutations). Non-limiting examples of GP130^{null} mutations include nonsense mutations (e.g., premature STOP codons) that prevent translation of functional gp130, missense mutations (e.g., substitutions) that reduce or abrogate gp130function, and frameshift mutations that prevent translation of a functional gp130polypeptide (e.g., by shortening the encoded amino acid sequence, or by adding, removing, or substituting one or more amino acid residues in a manner that reduces or abrogates gp130function). Nonsense, missense, and frameshift mutations are known in the art, as are methods of determining whether a given mutation impacts gp130 function. See, e.g., Schwerd et al., J Exp Med. 2017. 214 (9): 2547-2562.

In some embodiments, a method comprises modifying an endogenous GP130 promoter or portion thereof, such that less gp130-encoding RNA is transcribed in cells containing the modified GP130 promoter. In some embodiments, the endogenous GP130 promoter is modified at both GP130 alleles in the cell genome. In some embodiments, both GP130 allele promoters are modified in the same manner (e.g., to contain the same modified promoter sequence). In other embodiments, both promoters are modified to contain different sequences, each of which differs from the endogenous promoter sequence.

In some embodiments, a method comprises excising the endogenous GP130 promoter from one or both GP130 alleles of a genome. For example, a donor template comprising homology arms with homology to sequences upstream and downstream from the endogenous promoter, but lacking the promoter sequence, may be introduced into a cell, to promote replacement of the endogenous promoter with the donor template by homologous recombination, such that modified chromosome does not comprise an endogenous GP130 promoter. In some embodiments, the GP 130 promoter is excised from both GP130 alleles.

In some embodiments, a nuclease known in the art or described herein is used to cleave one or both chromosomes to promote modification, replacement, or excision of the endogenous GP130 promoter by homology-directed repair. In some embodiments, a nuclease known in the art or described herein is used to cleave one or both chromosomes to promote introduction of a GP130^{null} mutation into the cell genome. In some embodiments, an RNA-guided nuclease is used.

### In Vivo Methods

Some embodiments of producing Tregs with stable suppressive function are performed in vivo by administering to a subject reagents and/or compositions that induce or upregulate the TFG-β pathway in cells (e.g., immune cells of the subject such as CD3+,

CD4+ or CD8+) by expressing or inducing expression and/or activity of positive regulators of the TGF-β pathway (e.g., TGFβR, TGFβRII, Smad2, Smad3, or Smad4) and/or downregulating expression and/or activity of negative regulators of the TGF-β pathway (e.g., Smad7, IL-6R, gp130). In some embodiments, a gene encoding a constitutively active form of one or more positive regulators of the TFG-β pathway (e.g., TGFβRI, TGFβRII, Smad2, Smad3, and/or Smad4) is expressed in a cell, e.g., by delivery of nucleic acids comprising the gene into a subject. In some embodiments, a method of producing Tregs with stable suppressive function in vivo comprises gene editing of a gene involved in TGF-β signaling. In some embodiments, a gene involved in TGF-β signaling is a positive regulator and gene editing is performed to induce production of a constitutively active form of the positive regulator. In some embodiments, a gene involved in TGF-β signaling is a negative regulator and gene editing is performed to block or reduce expression of the negative regulator.

Compositions to administer may include nucleic acids, e.g., comprised in vectors (e.g., viral or non-viral vectors) or formulated using nanoparticles, that encode constitutively active positive regulators of the TGF-β pathway or inhibitors of negative regulators of the TGF-β pathway.

### Ex Vivo Methods

Some embodiments of methods of producing Tregs with stable suppressive function are performed ex vivo, e.g., in isolated immune cells that are transfected with, contacted with, or treated with reagents and/or compositions that induce or upregulate the TFG-β pathway in cells (e.g., immune cells of the subject such as CD3+, CD4+ or CD8+ cells) by expressing or inducing expression and/or activity of positive regulators of the TGF-β pathway (e.g., TGFβRI, TGFβRII, Smad2, Smad3, or Smad4) and/or downregulating expression and/or activity of negative regulators of the TGF-β pathway (e.g., Smad7, IL-6R, and/or gp130). Cells thus engineered are then administered to a subject. In some embodiments, a gene encoding a constitutively active form of one or more positive regulators of the TFG-β pathway (e.g., TGFβRI, TGFβRII, Smad2, Smad3, and/or Smad4) is expressed in a cell, e.g., by delivery of nucleic acids comprising the gene into a subject. In some embodiments, a method of producing Tregs with stable suppressive function ex vivo comprises gene editing of a gene involved in TGF-β signaling. In some embodiments, a gene involved in TGF-β signaling is a positive regulator and gene editing is performed to induce production of a constitutively active form of the positive regulator. In some embodiments, a gene involved in TGF-β signaling is a negative regulator and gene editing is performed to block or reduce expression of the negative regulator.

### Cells Expressing an Exogenous Coding Sequence

Some aspects of the disclosure relate to cells comprising a heterologous promoter operably linked to a coding sequence encoding TGFβRI, Smad2, or Smad3. In some embodiments, the coding sequence is a cDNA encoding TGFβRI, Smad2, or Smad3.

### TGF-β Receptor I Expression

In some embodiments, the cell comprises a heterologous promoter operably linked to a coding sequence (e.g., cDNA sequence) encoding TGFβRI or a functional derivative thereof.

In some embodiments, the encoded TGFβRI comprises one or more substitutions of amino acids corresponding to a GS domain of wild-type TGFβRI. In some embodiments, the encoded TGFβRI comprises one or more substitutions in the amino acid sequence corresponding to TTSGSGSG (SEQ ID NO: 23) of wild-type TGFβRI. The functional derivative of TGFβRI may include a protein that has a substantial activity of a wild-type TGFβRI, or increased activity relative to wild-type TGFβRI. One of ordinary skill in the art may use any method known in the art (e.g., phosphorylation assays and/or assaying changes in gene expression following stimulation with TGF-β) to test the functionality or activity of a TGFβRI or derivative thereof. The functional derivative of TGFβRI may also include any TGFβRI or fragment thereof that has conservative substitutions of one or more amino acid residues relative to full-length, wild-type TGFβRI as set forth in SEQ ID NO: 10 (UniProt Accession No. P36897).

In some embodiments, the encoded TGFβRI or functional derivative thereof has about or at least 30%, 40%, 50%, 60%, 70%, 80%, 85%, 90%, 95%, 96%, 97%, 98%, 99%, 100%, or more of the activity exhibited by a wild-type TGFβRI. In some embodiments, the encoded TGFβRI comprises an amino acid sequence with at least 80%, 85%, 90%, 91%, 92%, 93%, 94%, 95%, 96%, 97%, 98%, 99%, or up to 100% sequence identity to the amino acid sequence of wild-type human TGFβRI set forth as SEQ ID NO: 10. In some embodiments, the encoded TGFβRI comprises the wild-type amino acid sequence of SEQ ID NO: 10. In some embodiments, the encoded TGFβRI consists of the wild-type amino acid sequence of SEQ ID NO: 10. In other embodiments, the encoded TGFβRI comprises the amino acid sequence of SEQ ID NO: 11. In some embodiments, the encoded TGFβRI consists of the amino acid sequence of SEQ ID NO: 11.

In some embodiments, the nucleic acid sequence encoding TGFβRI comprises at least 60%, 70%, 75%, 80%, 85%, 90%, 91%, 92%, 93%, 94%, 95%, 96%, 97%, 98%, 99%, or up to 100% sequence identity to the nucleic acid sequence encoding wild-type human TGFβRI set forth as SEQ ID NO: 1. In some embodiments, the nucleic acid sequence encoding TGFβRI comprises the nucleic acid sequence of SEQ ID NO: 1. In some embodiments, the nucleic acid sequence encoding TGFβRI comprises the nucleic acid sequence of SEQ ID NO: 2. In some embodiments, the coding sequence is codon-optimized for expression in a cell. In some embodiments, the coding sequence is codon-optimized for expression in a human cell. In some embodiments, the nucleic acid sequence encoding TGFβRI comprises the nucleic acid sequence of SEQ ID NO: 3.

In some embodiments, the encoded TGFβRI comprises one or more substitutions of amino acids corresponding to a GS domain of wild-type TGFβRI. The “GS domain” of a TGFβRI refers to a glycine-(G) and serine(S)-rich domain in wild-type TGFβRI that precedes the kinase domain. For example, the amino acid sequence set forth by UniProt Accession No. P36897 includes, at amino acids 185-192, the amino acid sequence TTSGSGSG (SEQ ID NO: 23), and each residue of TTSGSGSG (SEQ ID NO: 23) is considered an amino acid corresponding to a GS domain of wild-type TGFβRI. In some embodiments, the TGFβRI comprises a T204D substitution. In some embodiments, the TGFβRI comprises a T204E substitution, which may also render the kinase domain of TGFβRI constitutively active. In some embodiments, the TGFβRI comprises an extracellular domain, a transmembrane domain, and a cytoplasmic domain. In some embodiments, the extracellular domain comprises at least 90%, at least 95%, at least 97%, at least 98%, at least 99%, or up to 100% sequence identity to the amino acid sequence set forth as SEQ ID NO: 15. In some embodiments, the transmembrane domain comprises at least 90%, at least 95%, at least 97%, at least 98%, at least 99%, or up to 100% sequence identity to the amino acid sequence set forth as SEQ ID NO: 16, In some embodiments, the transmembrane domain comprises at least 90%, at least 95%, at least 97%, at least 98%, at least 99%, or up to 100% sequence identity to the amino acid sequence set forth as SEQ ID NO: 17. In some embodiments, the transmembrane domain comprises at least 90%, at least 95%, at least 97%, at least 98%, at least 99%, or up to 100% sequence identity to the amino acid sequence set forth as SEQ ID NO: 17, and comprises an aspartate or glutamate at a position corresponding to amino acid 56 of SEQ ID NO: 17 (Thr204 in wild-type TGFβRI). In some embodiments, the transmembrane domain comprises at least 90%, at least 95%, at least 97%, at least 98%, at least 99%, or up to 100% sequence identity to the amino acid sequence set forth as SEQ ID NO: 18. In some embodiments, the TGFβRI further comprises a signal peptide. The signal peptide may be any signal peptide known in the art, such as a wild-type TGFβRI signal peptide having an amino acid sequence set forth in SEQ ID NO: 14, or a different signal peptide (e.g., a CD8 signal peptide).

In some embodiments, the encoded TGFβRI comprises one or more substitutions in the amino acid sequence corresponding to TTSGSGSG (SEQ ID NO: 23) of wild-type TGFβRI. In some embodiments, 1, 2, 3, 4, 5, 6, 7, or 8 amino acids corresponding to the GS domain are substituted. In some embodiments, 1, 2, or 3 amino acids corresponding to serine residues are substituted. In some embodiments, 1, 2, or 3 amino acids corresponding to glycine residues are substituted. In some embodiments, each serine residue of the GS domain is substituted. In some embodiments, each serine residue of the GS domain is substituted with the same amino acid. In some embodiments, each glycine residue of the GS domain is substituted. In some embodiments, each glycine residue of the GS domain is substituted with the same amino acid. In some embodiments, each amino acid corresponding to TTSGSGSG (SEQ ID NO: 23) is substituted. In some embodiments, each amino acid corresponding to TTSGSGSG (SEQ ID NO: 23) is substituted with the same amino acid. In some embodiments, an amino acid sequence corresponding to TTSGSGSG (SEQ ID NO: 23) is absent from the encoded TGFβRI (e.g., aligning the encoded TGFβRI amino acid sequence to a wild-type TGFβRI sequence shows a gap corresponding to TTSGSGSG (SEQ ID NO: 23)).

### Smad2 Expression

In some embodiments, the cell comprises a heterologous promoter operably linked to a coding sequence (e.g., cDNA sequence) encoding Smad2 or a functional derivative thereof. The functional derivative of Smad2 may include a protein that has a substantial activity of a wild-type Smad2, or increased activity relative to wild-type Smad2. One of ordinary skill in the art may use any method known in the art (e.g., phosphorylation assays and/or assaying changes in gene expression following stimulation with TGF-β) to test the functionality or activity of a Smad2 or derivative thereof. The functional derivative of Smad2 may also include any Smad2 or fragment thereof that has conservative substitutions of one or more amino acid residues relative to full-length, wild-type Smad2 as set forth in SEQ ID NO: 19 (UniProt Accession No. Q15796).

In some embodiments, the encoded Smad2 or functional derivative thereof has about or at least 30%, 40%, 50%, 60%, 70%, 80%, 85%, 90%, 95%, 96%, 97%, 98%, 99%, 100%, or more of the activity exhibited by a wild-type Smad2. In some embodiments, the encoded Smad2 comprises an amino acid sequence with at least 80%, 85%, 90%, 91%, 92%, 93%, 94%, 95%, 96%, 97%, 98%, 99%, or up to 100% sequence identity to the amino acid sequence of wild-type human Smad2 set forth as SEQ ID NO: 19. In some embodiments, the encoded Smad2 comprises the wild-type amino acid sequence of SEQ ID NO: 19. In some embodiments, the encoded Smad2 consists of the wild-type amino acid sequence of SEQ ID NO: 19. In other embodiments, the encoded Smad2 comprises the amino acid sequence of SEQ ID NO: 20. In some embodiments, the encoded Smad2 consists of the amino acid sequence of SEQ ID NO: 20.

In some embodiments, the nucleic acid sequence encoding Smad2 comprises at least 60%, 70%, 75%, 80%, 85%, 90%, 91%, 92%, 93%, 94%, 95%, 96%, 97%, 98%, 99%, or up to 100% sequence identity to the nucleic acid sequence encoding wild-type human Smad2 set forth as SEQ ID NO: 4. In some embodiments, the nucleic acid sequence encoding Smad2 comprises the nucleic acid sequence of SEQ ID NO: 4. In some embodiments, the nucleic acid sequence encoding Smad2 comprises the nucleic acid sequence of SEQ ID NO: 5. In some embodiments, the coding sequence is codon-optimized for expression in a cell. In some embodiments, the coding sequence is codon-optimized for expression in a human cell. In some embodiments, the nucleic acid sequence encoding Smad2 comprises the nucleic acid sequence of SEQ ID NO: 6.

In some embodiments, the encoded Smad2 comprises one or more substitutions of amino acids corresponding to one or more C-terminal serines of wild-type Smad2. “C-terminal serines of wild-type Smad2” refer to serine residues in an amino acid sequence that correspond (e.g., align) to amino acids 464-467 of wild-type human Smad2. For example, the amino acid sequence set forth by UniProt Accession No. Q15796 includes, at the C-terminus of its amino acid sequence, SSMS (SEQ ID NO: 24), and each serine residue of SEQ ID NO: 24 is considered a C-terminal serine of wild-type Smad2.

In some embodiments, the encoded Smad2 comprises one or more substitutions in an amino acid sequence corresponding to C-terminal Ser-Ser-X-Ser phosphorylation motif of wild-type Smad2. In some embodiments, one or more amino acids corresponding to Ser464, Ser465, or Ser467 of wild-type Smad2 are substituted in the encoded Smad2. In some embodiments, each of the serines in the Ser-Ser-X-Ser phosphorylation motif of wild-type Smad2 is substituted with an aspartate in the encoded Smad2 (e.g., the encoded Smad2 comprises the amino acid sequence Asp-Asp-X-Asp at amino acids corresponding to amino acids 464-467 of wild-type Smad2). In some embodiments, each of the serines is substituted with a glutamate. In some embodiments, each of the serines is substituted with a glutamate or aspartate.

### Smad3 Expression

In some embodiments, the cell comprises a heterologous promoter operably linked to a coding sequence (e.g., cDNA sequence) encoding Smad3 or a functional derivative thereof. The functional derivative of Smad3 may include a protein that has a substantial activity of a wild-type Smad3, or increased activity relative to wild-type Smad3. One of ordinary skill in the art may use any method known in the art (e.g., phosphorylation assays and/or assaying changes in gene expression following stimulation with TGF-β) to test the functionality or activity of a Smad3 or derivative thereof. The functional derivative of Smad3 may also include any Smad3 or fragment thereof that has conservative substitutions of one or more amino acid residues relative to full-length, wild-type Smad3 as set forth in SEQ ID NO: 21 (UniProt Accession No. P84022).

In some embodiments, the encoded Smad3 or functional derivative thereof has about or at least 30%, 40%, 50%, 60%, 70%, 80%, 85%, 90%, 95%, 96%, 97%, 98%, 99%, 100%, or more of the activity exhibited by a wild-type Smad3. In some embodiments, the encoded

Smad3 comprises an amino acid sequence with at least 80%, 85%, 90%, 91%, 92%, 93%, 94%, 95%, 96%, 97%, 98%, 99%, or up to 100% sequence identity to the amino acid sequence of wild-type human Smad3 set forth as SEQ ID NO: 21. In some embodiments, the encoded Smad3 comprises the wild-type amino acid sequence of SEQ ID NO: 21. In some embodiments, the encoded Smad3 consists of the wild-type amino acid sequence of SEQ ID NO: 21. In other embodiments, the encoded Smad3 comprises the amino acid sequence of SEQ ID NO: 22. In some embodiments, the encoded Smad3 consists of the amino acid sequence of SEQ ID NO: 22.

In some embodiments, the nucleic acid sequence encoding Smad3 comprises at least 60%, 70%, 75%, 80%, 85%, 90%, 91%, 92%, 93%, 94%, 95%, 96%, 97%, 98%, 99%, or up to 100% sequence identity to the nucleic acid sequence encoding wild-type human Smad3 set forth as SEQ ID NO: 7. In some embodiments, the nucleic acid sequence encoding Smad3 comprises the nucleic acid sequence of SEQ ID NO: 7. In some embodiments, the nucleic acid sequence encoding Smad3 comprises the nucleic acid sequence of SEQ ID NO: 8. In some embodiments, the coding sequence is codon-optimized for expression in a cell. In some embodiments, the coding sequence is codon-optimized for expression in a human cell. In some embodiments, the nucleic acid sequence encoding Smad3 comprises the nucleic acid sequence of SEQ ID NO: 9.

In some embodiments, the encoded Smad3 comprises one or more substitutions of terminal serines of wild-type Smad3″ refer to serine residues in an amino acid sequence that correspond (e.g., align) to amino acids 421-425 of wild-type human Smad3. For example, the amino acid sequence set forth by UniProt Accession No. P84022 includes, at the C-terminus of its amino acid sequence, CSSVS (SEQ ID NO: 25), and each serine residue of SEQ ID NO: 25 is considered a C-terminal serine of wild-type Smad3.

In some embodiments, the encoded Smad3 comprises one or more substitutions in an amino acid sequence corresponding to C-terminal Ser-Ser-X-Ser phosphorylation motif of wild-type Smad3. In some embodiments, one or more amino acids corresponding to Ser422, Ser423, or Ser425 of wild-type Smad3 are substituted in the encoded Smad3. In some embodiments, each of the serines in the Ser-Ser-X-Ser phosphorylation motif of wild-type Smad3 is substituted with an aspartate in the encoded Smad3 (e.g., the Smad3 comprises the amino acid sequence Asp-Asp-X-Asp at amino acids corresponding to amino acids 422-425 of wild-type Smad3). In some embodiments, each of the serines is substituted with a glutamate. In some embodiments, each of the serines is substituted with a glutamate or aspartate.

### Inserted Locus

In some embodiments, the heterologous promoter and coding sequence encoding TGFβRI, Smad2, or Smad3, or functional derivative thereof, are located in a locus corresponding to the polypeptide encoded by the coding sequence (e.g., a heterologous promoter and coding sequence encoding TGFβRI are inserted in a TGFβRI locus of the cell genome). In some embodiments, the cell comprises a heterologous promoter operably linked to a cDNA sequence encoding TGFβRI or a derivative thereof, integrated at a TGFβRI locus. In some embodiments, the cell comprises a heterologous promoter operably linked to a cDNA sequence encoding Smad2 or a derivative thereof, integrated at a SMAD2 locus. In some embodiments, the cell comprises a heterologous promoter operably linked to a cDNA sequence encoding Smad3 or a derivative thereof, integrated at a SMAD3 locus.

In some embodiments, the heterologous promoter and coding sequence encoding TGFβRI, Smad2, or Smad3, or functional derivative thereof, are located in a safe harbor locus. In some embodiments, the safe harbor locus is a HIPP11 locus. In some embodiments, the safe harbor locus is an AAVS1 locus. In some embodiments, the safe harbor locus is a ROSA26 locus.

In some embodiments, the heterologous promoter and coding sequence encoding TGFβRI, Smad2, or Smad3, or functional derivative thereof, are located in a T cell receptor locus. In some embodiments, the T cell receptor locus is a TRAC locus. In some embodiments, the T cell receptor locus is a TRBC locus.

### Cells for Stabilized Expression of an Endogenous Coding Sequence

Some aspects of the disclosure relate to cells comprising a heterologous promoter inserted into a nucleic acid (e.g., chromosome) of the cell genome, upstream from a coding sequence of an endogenous TGFβRI, SMAD2, or SMAD3 gene, such that the inserted promoter is operably linked to a coding sequence encoding TGFβRI, Smad2, or Smad3.

In some embodiments, the heterologous promoter is inserted into the genome at the endogenous promoter. Insertion of a heterologous promoter into the endogenous promoter may entirely remove endogenous promoter, or inactivate the endogenous promoter, depending on the sequence of the donor polynucleotide used in homology-directed repair. For example, a donor template may comprise, in 5′-to-3′ order, a sequence corresponding to a portion of the endogenous promoter, the heterologous promoter, and a sequence corresponding to a sequence downstream from the endogenous promoter, which, when incorporated by homology-directed repair, creates a chromosome containing the portion of the endogenous promoter followed by the inserted heterologous promoter.

In some embodiments, the heterologous promoter is inserted downstream from the endogenous promoter, and upstream from the first coding exon of the TGFβRI, SMAD2, or SMAD3 coding sequence. The heterologous promoter may be inserted at any position between the endogenous promoter and the first coding exon of the TGFβRI, SMAD2, or SMAD3 coding sequence. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides downstream from the endogenous promoter of TGFβRI, SMAD2, or SMAD3. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides upstream from the first coding exon of the TGFβRI, SMAD2, or SMAD3 coding sequence.

In some embodiments, the inserted promoter is active promoting transcription of RNA encoding TGFβRI, Smad2, or Smad3, even under pro-inflammatory conditions. In some embodiments, the inserted promoter is a constitutive promoter. In some embodiments, the constitutive promoter is an EF-1a, PGK, or MND promoter. In some embodiments, the constitutive promoter is an MND promoter. In some embodiments, the inserted promoter is an inducible promoter.

In some embodiments, the heterologous promoter is inserted into an exon of the TGFβRI, SMAD2, or SMAD3 coding sequence, thereby creating a synthetic exon. In such embodiments, the coding sequence may be modified relative to the endogenous sequence, but still capable of encoding a TGFβRI, Smad2, or Smad3 polypeptide. For example, one or more codons of the endogenous coding sequence may be replaced by nucleotides of the inserted promoter, and a downstream codon may be replaced by a start (AUG) codon, such that the inserted promoter mediates transcription of an mRNA that encodes a modified TGFβRI, Smad2, or Smad3 that is shorter than the endogenous form.

### TGF-β Receptor I Expression

In some embodiments, the heterologous promoter is inserted into the TGFβRI locus, such that the inserted promoter is operably linked to a coding sequence of the endogenous TGFβRI gene. In some embodiments, the endogenous TGFβRI coding sequence is modified, such that the TGFβRI encoded by the modified TGFβRI coding sequence comprises one or more amino acid substitutions relative to a wild-type TGFβRI (e.g., the wild-type TGFβRI amino acid sequence set forth in SEQ ID NO: 10 (UniProt Accession No. P36897)).

In some embodiments, the modified coding sequence encodes a functional derivative of TGFβRI. The functional derivative of TGFβRI may include a protein that has a substantial activity of a wild-type TGFβRI, or increased activity relative to wild-type TGFβRI. One of ordinary skill in the art may use any method known in the art (e.g., phosphorylation assays and/or assaying changes in gene expression following stimulation with TGF-β) to test the functionality or activity of a TGFβRI or derivative thereof. The functional derivative of TGFβRI may also include any TGFβRI or fragment thereof that has conservative substitutions of one or more amino acid residues relative to full-length, wild-type TGFβRI as set forth in SEQ ID NO: 10 (UniProt Accession No. P36897).

In some embodiments, the encoded TGFβRI or functional derivative thereof has about or at least 30%, 40%, 50%, 60%, 70%, 80%, 85%, 90%, 95%, 96%, 97%, 98%, 99%, 100%, or more of the activity exhibited by a wild-type TGFβRI. In some embodiments, the encoded TGFβRI comprises an amino acid sequence with at least 80%, 85%, 90%, 91%, 92%, 93%, 94%, 95%, 96%, 97%, 98%, 99%, or up to 100% sequence identity to the amino acid sequence of wild-type human TGFβRI set forth as SEQ ID NO: 10. In some embodiments, the encoded TGFβRI comprises the wild-type amino acid sequence of SEQ ID NO: 10. In some embodiments, the encoded TGFβRI consists of the wild-type amino acid sequence of SEQ ID NO: 10. In other embodiments, the encoded TGFβRI comprises the amino acid sequence of SEQ ID NO: 11. In some embodiments, the encoded TGFβRI consists of the amino acid sequence of SEQ ID NO: 11.

In some embodiments, the modified nucleic acid sequence encoding TGFβRI comprises at least 60%, 70%, 75%, 80%, 85%, 90%, 91%, 92%, 93%, 94%, 95%, 96%, 97%, 98%, 99%, or up to 100% sequence identity to the nucleic acid sequence encoding wild-type human TGFβRI set forth as SEQ ID NO: 1. In some embodiments, the modified nucleic acid sequence encoding TGFβRI comprises the nucleic acid sequence of SEQ ID NO: 1. In some embodiments, the modified nucleic acid sequence encoding TGFβRI comprises the nucleic acid sequence of SEQ ID NO: 2. In some embodiments, the coding sequence is codon-optimized for expression in a cell. In some embodiments, the coding sequence is codon-optimized for expression in a human cell. In some embodiments, the modified nucleic acid sequence encoding TGFβRI comprises the nucleic acid sequence of SEQ ID NO: 3.

In some embodiments, the encoded TGFβRI comprises one or more substitutions of amino acids corresponding to a GS domain of wild-type TGFβRI. The “GS domain” of a TGFβRI refers to a glycine-(G) and serine(S)-rich domain in wild-type TGFβRI that precedes the kinase domain. For example, the amino acid sequence set forth by UniProt Accession No. P36897 includes, at amino acids 185-192, the amino acid sequence TTSGSGSG (SEQ ID NO: 23), and each residue of TTSGSGSG (SEQ ID NO: 23) is considered an amino acid corresponding to a GS domain of wild-type TGFβRI. In some embodiments, the TGFβRI comprises a T204D substitution. In some embodiments, the TGFβRI comprises a T204E substitution, which may also render the kinase domain of TGFβRI constitutively active. In some embodiments, the TGFβRI comprises an extracellular domain, a transmembrane domain, and a cytoplasmic domain. In some embodiments, the extracellular domain comprises at least 90%, at least 95%, at least 97%, at least 98%, at least 99%, or up to 100% sequence identity to the amino acid sequence set forth as SEQ ID NO: 15. In some embodiments, the transmembrane domain comprises at least 90%, at least 95%, at least 97%, at least 98%, at least 99%, or up to 100% sequence identity to the amino acid sequence set forth as SEQ ID NO: 16, In some embodiments, the transmembrane domain comprises at least 90%, at least 95%, at least 97%, at least 98%, at least 99%, or up to 100% sequence identity to the amino acid sequence set forth as SEQ ID NO: 17. In some embodiments, the transmembrane domain comprises at least 90%, at least 95%, at least 97%, at least 98%, at least 99%, or up to 100% sequence identity to the amino acid sequence set forth as SEQ ID NO: 17, and comprises an aspartate or glutamate at a position corresponding to amino acid 56 of SEQ ID NO: 17 (Thr204 in wild-type TGFβRI). In some embodiments, the transmembrane domain comprises at least 90%, at least 95%, at least 97%, at least 98%, at least 99%, or up to 100% sequence identity to the amino acid sequence set forth as SEQ ID NO: 18. In some embodiments, the TGFβRI further comprises a signal peptide. The signal peptide may be any signal peptide known in the art, such as a wild-type TGFβRI signal peptide having an amino acid sequence set forth in SEQ ID NO: 14, or a different signal peptide (e.g., a CD8 signal peptide).

In some embodiments, the encoded TGFβRI comprises one or more substitutions in the amino acid sequence corresponding to TTSGSGSG (SEQ ID NO: 23) of wild-type TGFβRI. In some embodiments, 1, 2, 3, 4, 5, 6, 7, or 8 amino acids corresponding to the GS domain are substituted. In some embodiments, 1, 2, or 3 amino acids corresponding to serine residues are substituted. In some embodiments, 1, 2, or 3 amino acids corresponding to glycine residues are substituted. In some embodiments, each serine residue of the GS domain is substituted. In some embodiments, each serine residue of the GS domain is substituted with the same amino acid. In some embodiments, each glycine residue of the GS domain is substituted. In some embodiments, each glycine residue of the GS domain is substituted with the same amino acid. In some embodiments, each amino acid corresponding to TTSGSGSG (SEQ ID NO: 23) is substituted. In some embodiments, each amino acid corresponding to TTSGSGSG (SEQ ID NO: 23) is substituted with the same amino acid. In some embodiments, an amino acid sequence corresponding to TTSGSGSG (SEQ ID NO: 23) is absent from the encoded TGFβRI (e.g., aligning the encoded TGFβRI amino acid sequence to a wild-type TGFβRI sequence shows a gap corresponding to TTSGSGSG (SEQ ID NO: 23)).

### Smad2 Expression

In some embodiments, the heterologous promoter is inserted into the SMAD2 locus, such that the inserted promoter is operably linked to a coding sequence of the endogenous SMAD2 gene. In some embodiments, the endogenous SMAD2 coding sequence is modified, such that the Smad2 encoded by the modified SMAD2 coding sequence comprises one or more amino acid substitutions relative to a wild-type Smad2 (e.g., the wild-type Smad2 amino acid sequence set forth in SEQ ID NO: 19 (UniProt Accession No. Q15796)).

In some embodiments, the modified coding sequence encodes a functional derivative of Smad2. The functional derivative of Smad2 may include a protein that has a substantial activity of a wild-type Smad2, or increased activity relative to wild-type Smad2. One of ordinary skill in the art may use any method known in the art (e.g., phosphorylation assays and/or assaying changes in gene expression following stimulation with TGF-β) to test the functionality or activity of a Smad2 or derivative thereof. The functional derivative of Smad2 may also include any Smad2 or fragment thereof that has conservative substitutions of one or more amino acid residues relative to full-length, wild-type Smad2 as set forth in SEQ ID NO: 19 (UniProt Accession No. Q15796).

In some embodiments, the encoded Smad2 or functional derivative thereof has about or at least 30%, 40%, 50%, 60%, 70%, 80%, 85%, 90%, 95%, 96%, 97%, 98%, 99%, 100%, or more of the activity exhibited by a wild-type Smad2. In some embodiments, the encoded Smad2 comprises an amino acid sequence with at least 80%, 85%, 90%, 91%, 92%, 93%, 94%, 95%, 96%, 97%, 98%, 99%, or up to 100% sequence identity to the amino acid sequence of wild-type human Smad2 set forth as SEQ ID NO: 19. In some embodiments, the encoded Smad2 comprises the wild-type amino acid sequence of SEQ ID NO: 19. In some embodiments, the encoded Smad2 consists of the wild-type amino acid sequence of SEQ ID NO: 19. In other embodiments, the encoded Smad2 comprises the amino acid sequence of SEQ ID NO: 20. In some embodiments, the encoded Smad2 consists of the amino acid sequence of SEQ ID NO: 20.

In some embodiments, the nucleic acid sequence encoding Smad2 comprises at least 60%, 70%, 75%, 80%, 85%, 90%, 91%, 92%, 93%, 94%, 95%, 96%, 97%, 98%, 99%, or up to 100% sequence identity to the nucleic acid sequence encoding wild-type human Smad2 set forth as SEQ ID NO: 4. In some embodiments, the nucleic acid sequence encoding Smad2 comprises the nucleic acid sequence of SEQ ID NO: 4. In some embodiments, the nucleic acid sequence encoding Smad2 comprises the nucleic acid sequence of SEQ ID NO: 5. In some embodiments, the coding sequence is codon-optimized for expression in a cell. In some embodiments, the coding sequence is codon-optimized for expression in a human cell. In some embodiments, the nucleic acid sequence encoding Smad2 comprises the nucleic acid sequence of SEQ ID NO: 6.

In some embodiments, the encoded Smad2 comprises one or more substitutions of amino acids corresponding to one or more C-terminal serines of wild-type Smad2. “C-terminal serines of wild-type Smad2” refer to serine residues in an amino acid sequence that correspond (e.g., align) to amino acids 464-467 of wild-type human Smad2. For example, the amino acid sequence set forth by UniProt Accession No. Q15796 includes, at the C-terminus of its amino acid sequence, SSMS (SEQ ID NO: 24), and each serine residue of SEQ ID NO: 24 is considered a C-terminal serine of wild-type Smad2.

In some embodiments, the encoded Smad2 comprises one or more substitutions in an amino acid sequence corresponding to C-terminal Ser-Ser-X-Ser phosphorylation motif of wild-type Smad2. In some embodiments, one or more amino acids corresponding to Ser464, Ser465, or Ser467 of wild-type Smad2 are substituted in the encoded Smad2. In some embodiments, each of the serines in the Ser-Ser-X-Ser phosphorylation motif of wild-type Smad2 is substituted with an aspartate in the encoded Smad2 (e.g., the encoded Smad2 comprises the amino acid sequence Asp-Asp-X-Asp at amino acids corresponding to amino acids 464-467 of wild-type Smad2). In some embodiments, each of the serines is substituted with a glutamate. In some embodiments, each of the serines is substituted with a glutamate or aspartate.

### Smad3 Expression

In some embodiments, the heterologous promoter is inserted into the SMAD3 locus, such that the inserted promoter is operably linked to a coding sequence of the endogenous SMAD3 gene. In some embodiments, the SMAD3 coding sequence is modified relative to an endogenous SMAD3 gene. Such modifications may remove one or more introns and/or mutate one or more exons of the endogenous SMAD3 gene. In some embodiments, the endogenous SMAD3 coding sequence is modified, such that the Smad3 encoded by the modified SMAD3 coding sequence comprises one or more amino acid substitutions relative to a wild-type Smad3 (e.g., the wild-type Smad3 amino acid sequence set forth in SEQ ID NO: 21 (UniProt Accession No. P84022)).

In some embodiments, the modified coding sequence encodes a functional derivative of Smad3. The functional derivative of Smad3 may include a protein that has a substantial activity of a wild-type Smad3, or increased activity relative to wild-type Smad3. One of ordinary skill in the art may use any method known in the art (e.g., phosphorylation assays and/or assaying changes in gene expression following stimulation with TGF-β) to test the functionality or activity of a Smad3 or derivative thereof. The functional derivative of Smad3 may also include any Smad3 or fragment thereof that has conservative substitutions of one or more amino acid residues relative to full-length, wild-type Smad3 as set forth in SEQ ID NO: 21 (UniProt Accession No. P84022).

In some embodiments, the encoded Smad3 or functional derivative thereof has about or at least 30%, 40%, 50%, 60%, 70%, 80%, 85%, 90%, 95%, 96%, 97%, 98%, 99%, 100%, or more of the activity exhibited by a wild-type Smad3. In some embodiments, the encoded Smad3 comprises an amino acid sequence with at least 80%, 85%, 90%, 91%, 92%, 93%, 94%, 95%, 96%, 97%, 98%, 99%, or up to 100% sequence identity to the amino acid sequence of wild-type human Smad3 set forth as SEQ ID NO: 21. In some embodiments, the encoded Smad3 comprises the wild-type amino acid sequence of SEQ ID NO: 21. In some embodiments, the encoded Smad3 consists of the wild-type amino acid sequence of SEQ ID NO: 21. In other embodiments, the encoded Smad3 comprises the amino acid sequence of SEQ ID NO: 22. In some embodiments, the encoded Smad3 consists of the amino acid sequence of SEQ ID NO: 22.

In some embodiments, the modified nucleic acid sequence encoding Smad3 comprises at least 60%, 70%, 75%, 80%, 85%, 90%, 91%, 92%, 93%, 94%, 95%, 96%, 97%, 98%, 99%, or up to 100% sequence identity to the nucleic acid sequence encoding wild-type human Smad3 set forth as SEQ ID NO: 7. In some embodiments, the modified nucleic acid sequence encoding Smad3 comprises the nucleic acid sequence of SEQ ID NO: 7. In some embodiments, the modified nucleic acid sequence encoding Smad3 comprises the nucleic acid sequence of SEQ ID NO: 8. In some embodiments, the coding sequence is codon-optimized for expression in a cell. In some embodiments, the coding sequence is codon-optimized for expression in a human cell. In some embodiments, the modified nucleic acid sequence encoding Smad3 comprises the nucleic acid sequence of SEQ ID NO: 9.

In some embodiments, the encoded Smad3 comprises one or more substitutions of amino acids corresponding to one or more C-terminal serines of wild-type Smad3. “C-terminal serines of wild-type Smad3” refer to serine residues in an amino acid sequence that correspond (e.g., align) to amino acids 421-425 of wild-type human Smad3. For example, the amino acid sequence set forth by UniProt Accession No. P84022 includes, at the C-terminus of its amino acid sequence, CSSVS (SEQ ID NO: 25), and each serine residue of SEQ ID NO: 25 is considered a C-terminal serine of wild-type Smad3.

In some embodiments, the encoded Smad3 comprises one or more substitutions in an amino acid sequence corresponding to C-terminal Ser-Ser-X-Ser phosphorylation motif of wild-type Smad3. In some embodiments, one or more amino acids corresponding to Ser422. Ser423, or Ser425 of wild-type Smad3 are substituted in the encoded Smad3. In some embodiments, each of the serines in the Ser-Ser-X-Ser phosphorylation motif of wild-type Smad3 is substituted with an aspartate in the encoded Smad3 (e.g., the Smad3 comprises the amino acid sequence Asp-Asp-X-Asp at amino acids corresponding to amino acids 422-425 of wild-type Smad3). In some embodiments, each of the serines is substituted with a glutamate. In some embodiments, each of the serines is substituted with a glutamate or aspartate.

Cells with Inhibited Smad7 and/or IL-6 Signaling

Some embodiments of cells described herein exhibit reduced expression of SMAD7, IL6R, and/or GP130. Such reductions in expression may be achieved through any method known in the art or described herein, such as in the foregoing section “Methods for inhibiting SMAD7 and/or IL-6 signaling.” For example, cells may contain RNAi molecules (e.g., siRNA, shRNA, miRNA) that reduce expression of Smad7, IL-6R, and/or gp130 proteins. The presence of such RNAi molecules may be achieved by introduction of the RNAi molecules into the cell, or by expression from a nucleic acid (e.g., an integrated nucleic acid in the genome or an episomal vector) to provide consistent regulation and/or inhibition of a targeted gene.

Some embodiments of cells described herein exhibit reduced expression of Smad7. Reduction of Smad7 expression may be reduced by any method known in the art, including inactivation or removal of promoters and/or coding sequences encoding Smad7, and/or expression of RNAi molecules (e.g., miRNA, siRNA, shRNA) that bind to and mediate degradation of mRNA encoding Smad7.

In some embodiments, an endogenous SMAD7 promoter (i.e., an endogenous promoter operably linked to a SMAD7 coding sequence) is replaced with a sequence that reduces transcription of Smad7-encoding RNA, relative to the endogenous promoter. In some embodiments, the endogenous promoter is replaced with an inactive promoter that does not promote transcription of Smad7-encoding RNA.

In some embodiments, a cell comprises a SMAD^{null} mutation in a SMAD7 allele of the genome. In some embodiments, each SMAD7 allele comprises a SMAD^{null} mutation. In some embodiments, a cell is homozygous for a SMAD7^{null} allele. Non-limiting examples of SMAD^{null} mutations include nonsense mutations (e.g., premature STOP codons) that prevent translation of functional Smad7, missense mutations (e.g., substitutions) that reduce or abrogate Smad7function, and frameshift mutations that prevent translation of a functional Smad7 polypeptide (e.g., by shortening the encoded amino acid sequence, or by adding, removing, or substituting one or more amino acid residues in a manner that reduces or abrogates Smad7 function). Nonsense, missense, and frameshift mutations are known in the art, as are methods of determining whether a given mutation impacts Smad7 function. See, e.g., Zhang et al., Mol Cell Biol. 2007. 27 (12): 4488-4499.

In some embodiments, the cell comprises a nucleic acid encoding an RNA interference (RNAi) molecule comprising a sequence that is complementary to a sequence in mRNA encoding SMAD7. In some embodiments, the RNAi molecule is miRNA. In some embodiments, the RNAi molecule is siRNA. In some embodiments, the RNAi molecule is shRNA. The nucleic acid encoding the RNAi molecule may be present on a vector (e.g., plasmid or viral vector) introduced into the cell. Additionally or alternatively, the nucleic acid comprising the inserted heterologous promoter may also comprise a nucleic acid sequence encoding the RNAi molecule, such that the heterologous promoter also drives expression of the RNAi molecule, resulting in targeted degradation of Smad7-encoding mRNA in the cell and reduced expression of Smad7 protein.

In some embodiments, the cell does not express detectable Smad7 protein. Detection of Smad7, and consequently a determination of whether a cell expresses detectable Smad7, may be accomplished by any method known in the art. Non-limiting examples of such methods include flow cytometry, western blotting, immunoprecipitation, and fluorescent microscopy. In some embodiments, the cell does not transcribe detectable SMAD7-encoding mRNA. Detection of Smad7-encoding mRNA, and consequently a determination of whether a cell expresses detectable Smad7-encoding mRNA, may be accomplished by any method known in the art. Non-limiting examples of such methods include qRT-PCR, RNAseq (e.g., single-cell RNAseq), fluorescent in situ hybridization, and probe-based microscopy.

Some embodiments of cells described herein exhibit reduced expression of IL-6R or gp130, such that the cell is less capable of responding to IL-6, Reduction of IL-6R or gp130 expression may be reduced by any method known in the art, including inactivation or removal of promoters and/or coding sequences encoding IL-6R or gp130, and/or expression of RNAi molecules (e.g., miRNA, siRNA, shRNA) that bind to and mediate degradation of mRNA encoding IL-6R or gp130.

In some embodiments, an endogenous IL6R promoter (i.e., an endogenous promoter operably linked to an IL6R coding sequence) is replaced with a sequence that reduces transcription of IL-6R-encoding RNA, relative to the endogenous promoter. In some embodiments, the endogenous promoter is replaced with an inactive promoter that does not promote transcription of IL-6R-encoding RNA. In some embodiments, an endogenous GP130 promoter (i.e., an endogenous promoter operably linked to an GP130 coding sequence) is replaced with a sequence that reduces transcription of gp130-encoding RNA, relative to the endogenous promoter. In some embodiments, the endogenous promoter is replaced with an inactive promoter that does not promote transcription of gp130-encoding RNA.

In some embodiments, a cell comprises an IL6R^{null} mutation in an IL6R allele of the genome. In some embodiments, each IL6R allele comprises an IL6R^{null} mutation. In some embodiments, a cell is homozygous for an IL6R^{null} allele. Non-limiting examples of IL6R^{null} mutations include nonsense mutations (e.g., premature STOP codons) that prevent translation of functional IL-6R, missense mutations (e.g., substitutions) that reduce or abrogate IL-6R function, and frameshift mutations that prevent translation of a functional IL-6R polypeptide (e.g., by shortening the encoded amino acid sequence, or by adding, removing, or substituting one or more amino acid residues in a manner that reduces or abrogates IL-6R function). Nonsense, missense, and frameshift mutations are known in the art, as are methods of determining whether a given mutation impacts IL-6R function. See, e.g., Spencer et al., J Exp Med. 2019. 216 (9): 1986-1998.

In some embodiments, a cell comprises a GP130^{null} mutation in a GP130 allele of the genome. In some embodiments, each GP130 allele comprises a GP130^{null} mutation. In some embodiments, a cell is homozygous for a GP130^{null} allele. Non-limiting examples of GP130^{null} mutations include nonsense mutations (e.g., premature STOP codons) that prevent translation of functional IL-6R, missense mutations (e.g., substitutions) that reduce or abrogate gp130 function, and frameshift mutations that prevent translation of a functional gp130 polypeptide (e.g., by shortening the encoded amino acid sequence, or by adding, removing, or substituting one or more amino acid residues in a manner that reduces or abrogates gp130 function). Nonsense, missense, and frameshift mutations are known in the art, as are methods of determining whether a given mutation impacts gp130 function. See, e.g., Schwerd et al., J Exp Med. 2017. 214 (9):2547-2562.

In some embodiments, the cell comprises a nucleic acid encoding an RNA interference (RNAi) molecule comprising a sequence that is complementary to a sequence in mRNA encoding IL-6R. In some embodiments, the RNAi molecule is miRNA. In some embodiments, the RNAi molecule is siRNA. In some embodiments, the RNAi molecule is shRNA. The nucleic acid encoding the RNAi molecule may be present on a vector (e.g., plasmid or viral vector) introduced into the cell. Additionally or alternatively, the nucleic acid comprising the inserted heterologous promoter may also comprise a nucleic acid sequence encoding the RNAi molecule, such that the heterologous promoter also drives expression of the RNAi molecule, resulting in targeted degradation of IL-6R-encoding mRNA in the cell and reduced expression of IL-6R protein.

In some embodiments, the cell comprises a nucleic acid encoding an RNA interference (RNAi) molecule comprising a sequence that is complementary to a sequence in mRNA encoding gp130. In some embodiments, the RNAi molecule is miRNA. In some embodiments, the RNAi molecule is siRNA. In some embodiments, the RNAi molecule is shRNA. The nucleic acid encoding the RNAi molecule may be present on a vector (e.g., plasmid or viral vector) introduced into the cell. Additionally or alternatively, the nucleic acid comprising the inserted heterologous promoter may also comprise a nucleic acid sequence encoding the RNAi molecule, such that the heterologous promoter also drives expression of the RNAi molecule, resulting in targeted degradation of gp130-encoding mRNA in the cell and reduced expression of gp130 protein.

In some embodiments, the cell does not express detectable IL-6R protein. Detection of IL-6R, and consequently a determination of whether a cell expresses detectable IL-6R, may be accomplished by any method known in the art. Non-limiting examples of such methods include flow cytometry, western blotting, immunoprecipitation, and fluorescent microscopy. In some embodiments, the cell does not transcribe detectable IL-6R-encoding mRNA. Detection of IL-6R-encoding mRNA, and consequently a determination of whether a cell expresses detectable IL-6R-encoding mRNA, may be accomplished by any method known in the art. Non-limiting examples of such methods include qRT-PCR, RNAseq (e.g., single-cell RNAseq), fluorescent in situ hybridization, and probe-based microscopy.

In some embodiments, the cell does not express detectable gp130 protein. Detection of gp130, and consequently a determination of whether a cell expresses detectable gp130, may be accomplished by any method known in the art. Non-limiting examples of such methods include flow cytometry, western blotting, immunoprecipitation, and fluorescent microscopy. In some embodiments, the cell does not transcribe detectable gp130-encoding mRNA. Detection of gp130-encoding mRNA, and consequently a determination of whether a cell expresses detectable gp130-encoding mRNA, may be accomplished by any method known in the art. Non-limiting examples of such methods include qRT-PCR, RNAseq (e.g., single-cell RNAseq), fluorescent in situ hybridization, and probe-based microscopy.

In some embodiments, the cell has a diminished ability to respond to IL-6 stimulation, compared to a cell in which IL-6R or gp130 expression are not reduced by a modification described herein. In some embodiments, the cell expresses FoxP3 even in the presence of IL-6, In some embodiments, the cell does not express detectable IL-17 following contact with IL-6.

### Nucleases

Aspects of the disclosure relate to the use of nucleases to introduce a double-stranded break into nucleic acid of a cell genome and edit the genome at a desired locus (e.g., to promote integration of a donor template at the locus by homology-directed repair and/or inactivate a targeted gene). Any one of multiple gene- or genome-editing methods can used to accomplish editing of one or more loci (e.g., TGFβRI, SMAD2, SMAD3, SMAD4, SMAD7, IL6R, GP130, TRAC, TRBC, AAVS1, and/or HIPP11). Non-limiting examples of gene editing methods include use of a DNA endonuclease such as an RNA-guided nuclease (e.g., Cas (e.g., Cas9) nuclease), zinc finger nuclease (ZFN), transcription activator-like effector nuclease (TALEN), or meganuclease; transposon-mediated gene editing; serine integrase-mediated gene editing; and lentivirus-mediated gene editing. In some embodiments, a gene editing method comprises knocking out or inactivating an endogenous gene, such as by producing a chromosomal gene knockout in the genome. As used herein, the term “chromosomal gene knockout” refers to a genetic alteration, inactivation, or introduced inhibitory agent in a host cell that prevents (e.g., reduces, delays, suppresses, or abrogates) production, by the host cell, of a functionally active endogenous polypeptide product. Alterations resulting in a chromosomal gene knockout or inactivation can include, for example, introduced nonsense mutations (including the formation of premature stop codons), missense mutations, gene deletion, or strand breaks.

In certain embodiments, a chromosomal gene knock-out or gene knock-in (e.g., insertion) is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. A DNA endonuclease refers to an endonuclease that is capable of catalyzing cleavage of a phosphodiester bond within a DNA polynucleotide. In certain embodiments, an endonuclease is capable of cleaving a nucleic acid sequence in a targeted gene, thereby inactivating or “knocking out” the targeted gene. In some embodiments, an endonuclease is capable of cleaving a nucleic acid sequence in a targeted locus, promoting insertion of an exogenous nucleic acid sequence into the targeted locus by homologous recombination. An endonuclease may be a naturally occurring, recombinant, genetically modified, or fusion endonuclease. Examples of endonucleases for use in gene editing include zinc finger nucleases (ZFN), TALE-nucleases (TALEN), RNA-guided nucleases, CRISPR-Cas nucleases, meganucleases, or megaTALs.

The nucleic acid strand breaks caused by DNA endonucleases are typically double-strand breaks (DSB), which may be commonly repaired through the distinct mechanisms of homology directed repair (HDR) by homologous recombination, or by non-homologous end joining (NHEJ). (NHEJ: Ghezraoui et al., 2014 Mol Cell 55 (6): 829-842; HDR: Jasin and Rothstein, 2013 Cold Spring Harb Perspect Biol 5 (11): a012740, PMID 24097900). During HDR/homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. HDR is favored by the presence of a donor template at the time of DSB formation.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair (HDR). Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout or inactivation comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histidine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparaginc) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair (HDR) can introduce a transgene at the site of DSB providing homologous flanking sequences are present in the donor template containing the transgene. In certain embodiments, a gene knockout comprises an insertion, a deletion, a mutation or a combination thereof, and made using a TALEN molecule.

Gene-editing systems and methods described herein may make use of viral or non-viral vectors or cassettes, as well as nucleases that allow site-specific or locus-specific gene-editing, such as RNA-guided nucleases, Cas nucleases (e.g., Cpf1 or Cas9 nucleases), meganucleases, TALENs, or ZFNs. Certain RNA-guided nucleases useful with some embodiments provided herein are disclosed in U.S. Pat. No. 11,162,114, which is expressly incorporated by reference herein in its entirety. Non-limiting examples of Cas nucleases include SpCas9, SaCas9, CjCas9, xCas9, C2c1, Cas13a/C2c2, C2c3, Cas13b, Cpf1, and variants thereof. Certain features useful with some embodiments provided herein are disclosed in WO 2019/210057, which is expressly incorporated by reference in its entirety.

As used herein, a “clustered regularly interspaced short palindromic repeats/Cas” (CRISPR/Cas, or Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into types (e.g., type I, type II, type III, and type V) based on the sequence and structure of the Cas nucleases. The crRNA-guided surveillance complexes in types I and III need multiple Cas subunits. The Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA: tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a donor template transgene with homologous flanking sequences can be introduced at the site of DSB via homology directed repair (HDR). The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9: e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). Non-limiting examples of CRISPR/Cas nucleases include Cas9, SaCas9, CjCas9, xCas9, C2C1, Cas13a/C2c2, C2c3, Cas13b, Cpf1, and variants thereof.

In some embodiments, a gene knockout or inactivation comprises an insertion, a deletion, a mutation or a combination thereof, and made using an RNA-guided nuclease. Exemplary gRNA sequences and methods of using the same to knock out endogenous genes that encode immune cell proteins include those described in Ren et al., Clin Cancer Res. 2017. 23 (9): 2255-2266, the gRNAs, Cas9 DNAs, vectors, and gene knockout techniques of which are hereby expressly incorporated by reference in their entirety.

In some embodiments, a gene modification comprises an insertion of an exogenous nucleic acid sequence (e.g., heterologous promoter, transgene, and/or combinations thereof) into the genome of a cell, where an RNA-guided nuclease introduces a double-stranded break in the genome and the exogenous nucleic acid sequence is introduced into the genome by homology-directed repair. In some embodiments, a gene modification comprises an inactivation of a target gene (e.g., IL6R, GP130, and/or SMAD7) in the genome of a cell, where an RNA-guided nuclease introduces a double-stranded break in the genome, and repair of the break by non-homologous end joining inactivates the gene.

### Heterologous Promoters and Uses Thereof

Embodiments of the compositions, cells, nucleic acids, vectors, and methods described herein that contemplate use of a promoter (e.g., heterologous promoter) may use any promoter known in the art. In some embodiments, the heterologous promoter on the introduced nucleic acid is active, promoting transcription of RNA encoding TGFβRI, Smad2, or Smad3, even under pro-inflammatory conditions. In some embodiments, the heterologous promoter is a constitutive promoter, which promotes transcription of an operably linked sequence (e.g., a TGFβRI, Smad2, or Smad3 polypeptide) at a consistent rate. Constitutive promoters may be strong promoters, which promote transcription at a higher rate than an endogenous promoter, or weak promoters, which promote transcription at a lower rate than a strong or endogenous promoter. In some embodiments, the constitutive promoter is a strong promoter. In some embodiments, the constitutive promoter is a weak promoter. In some embodiments, the constitutive promoter is an EF-1a, PGK, or MND promoter. In some embodiments, another promoter known in the art, such as an SV40, CMV, UBC, or CAGG promoter, is used. In some embodiments, the constitutive promoter is an MND promoter. In some embodiments, the heterologous promoter is an inducible promoter. Inducible promoters promote transcription of an operably linked sequence in response to the presence of an activating signal, or the absence of a repressor signal. In some embodiments, the inducible promoter is inducible by a drug or steroid.

### Cell Types

Embodiments of methods for producing genetically modified cells (e.g., by in vitro or ex vivo gene editing, and/or administration of compositions, vectors, or nucleic acids to a subject for in vivo editing) may use any cell type known in the art as a material for, e.g., introduction of nucleic acids, vectors, and/or compositions. It is to be understood that methods described herein that comprise manipulation of CD4+ cells, can be applied to other types of cells (e.g., CD8+ cells). In some embodiments, the methods described herein comprise editing an immune cell. Non-limiting examples of immune cells include B cells, T cells, and NK cells. In some embodiments, the methods provided herein comprise editing CD3+ cells, thereby producing edited CD3+ cells, including CD4+ and CD8+ Treg cells. In some embodiments, the methods comprise editing CD4+ T cells, thereby producing CD4+ Treg cells. In some embodiments, the methods comprise editing CD8+ T cells, thereby producing CD8+ Treg cells. In some embodiments, the methods comprise editing NK1.1+ T cells, thereby producing NK1.1+ Treg cells.

In some embodiments, the methods comprise editing a stem cell. In some embodiments, the methods comprise editing a pluripotent stem cell. In some embodiments, the methods comprise editing CD34+ hematopoietic stem cells (HSCs). In some embodiments, the methods comprise editing induced pluripotent stem cells (iPSCs). Edited stem cells may be matured in vitro to produce Treg cells, or administered to a subject to allow in vivo development into Treg cells. Edited stem cells may be matured into CD3+ Treg cells, CD4+ Treg cells, CD8+ Treg cells, NK1.1+ Treg cells, or a combination thereof.

In some embodiments, a method comprises editing a T cell. A T cell or T lymphocyte is an immune system cell that matures in the thymus and produces a T cell receptor (TCR), e.g., an antigen-specific heterodimeric cell surface receptor typically comprised of an α-β heterodimer or a γ-δ heterodimer. T cells of a given clonality typically express only a single TCR clonotype that recognizes a specific antigenic epitope presented by a syngeneic antigen-presenting cell in the context of a major histocompatibility complex-encoded determinant. T cells can be naïve (“TN”; not exposed to antigen; increased expression of CD62L, CCR7. CD28, CD3, CD127, and CD45RA, and decreased or no expression of CD45RO as compared to TCM (described herein)), memory T cells™ (antigen experienced and long-lived), including stem cell memory T cells, and effector cells (antigen-experienced, cytotoxic). TM can be further divided into subsets of central memory T cells (TCM, expresses CD62L, CCR7, CD28, CD95, CD45RO, and CD127) and effector memory T cells (TEM, express CD45RO, decreased expression of CD62L, CCR7, CD28, and CD45RA). Effector T cells (TE) refers to antigen-experienced CD8+ cytotoxic T lymphocytes that express CD45RA, have decreased expression of CD62L, CCR7, and CD28 as compared to TCM, and are positive for granzyme and perforin. Helper T cells (TH) are CD4+ cells that influence the activity of other immune cells by releasing cytokines. CD4+ T cells can activate and suppress an adaptive immune response, and which of those two functions is induced will depend on the presence of other cells and signals. T cells can be collected using known techniques, and the various subpopulations or combinations thereof can be enriched or depleted by known techniques, for example, using antibodies that specifically recognize one or more T cell surface phenotypic markers, by affinity binding to antibodies, flow cytometry, fluorescence activated cell sorting (FACS), or immunomagnetic bead selection. Other exemplary T cells include regulatory T cells (Treg, also known as suppressor T cells), such as CD4+CD25+ (FoxP3+) regulatory T cells and Treg17 cells, as well as Tr1, Th3, CD8+CD28−, or Qa-1 restricted T cells. In some embodiments, the cell is a CD3+, CD4+, and/or CD8+ T cell. In some embodiments, the cell is a CD3+ T cell. In some embodiments, the cell is a CD4⁺ CD8⁻ T cell. In some embodiments, the cell is a CD4 CD8+ T cell. In some embodiments, the cell is a regulatory T cell (Treg). Non-limiting examples of Treg cells are Tr1, Th3, CD8+CD28−, and Qa-1 restricted T cells. In some embodiments, the Treg cell is a FoxP3+ Treg cell. In some embodiments, the Treg cell expresses CTLA-4, LAG-3, CD25, CD39, CD27, CD70, CD357 (GITR), neuropilin-1, galectin-1, and/or IL-2Ra on its surface.

In some embodiments, the cell is a human cell. In some embodiments, a cell as described herein is isolated from a biological sample. A biological sample may be a sample from a subject (e.g., a human subject) or a composition produced in a lab (e.g., a culture of cells). A biological sample obtained from a subject make be a liquid sample (e.g., blood or a fraction thereof, a bronchial lavage, cerebrospinal fluid, or urine), or a solid sample (e.g., a piece of tissue) In some embodiments, the cell is obtained from peripheral blood. In some embodiments, the cell is obtained from umbilical cord blood. In some embodiments, the cell is65btainned by sorting cells of peripheral blood to 65btainn a desired cell population (e.g., CD3+ cells), and one or more cells of the sorted population are modified by a method described herein. Also contemplated herein are cells produced by a method described herein.

Embodiments of genetically modified cells described herein may be any cell type known in the art. In some embodiments, the cell is a T cell, a precursor T cell, or a hematopoietic stem cell. In some embodiments, the cell is an NK-T cell (e.g., a FoxP3-NK-T cell or a FoxP3+NK-T cell). In some embodiments, the cell is a regulatory B (Breg) cell (e.g., a FoxP3-B cell or a FoxP3+B cell). In some embodiments, the cell is a CD4+ T cell (e.g., a FoxP3-CD4+ T cell or a FoxP3+CD4+ T cell) or a CD8+ T cell (e.g., a FoxP3-CD8+ T cell or a FoxP3+CD8+ T cell). In some embodiments, the cell is a CD25-T cell. In some embodiments, the cell is a regulatory T (Treg) cell. Non-limiting examples of Treg cells are Tr1. Th3, CD8+CD28−, and Qa-1 restricted T cells. In some embodiments, the Treg cell is a FoxP3+ Treg cell. In some embodiments, the Treg cell expresses CTLA-4, LAG-3, CD25, CD39, CD27, CD70, CD357 (GITR), neuropilin-1, galectin-1, and/or IL-2Ra on its surface. In some embodiments, the Treg cell is TGFβRI+. In some embodiments, the Treg cell is Smad2+. In some embodiments, the Treg cell is Smad3+.

### Chemically Induced Signaling Complexes (CISC)

Some embodiments of the methods of modifying cells provided herein comprise introducing into the cell one or more nucleic acids that collectively comprise (1) a first nucleic acid sequence encoding a first chemically inducible signaling complex (CISC) component, and (2) a second nucleic acid sequence encoding a second chemically inducible signaling complex (CISC) component, each CISC component comprising (a) an extracellular binding domain that is capable of binding to a CISC inducer molecule, (b) a transmembrane domain, and (c) an intracellular signaling domain, such that binding of the first and second CISC components to the CISC inducer molecule results in dimerization of the CISC components and a signal transduction event in the cell. Similarly, some embodiments of cells described herein comprise (1) a first nucleic acid sequence encoding a first chemically inducible signaling complex (CISC) component, and (2) a second nucleic acid sequence encoding a second chemically inducible signaling complex (CISC) component, each CISC component comprising (a) an extracellular binding domain that is capable of binding to a CISC inducer molecule, (b) a transmembrane domain, and (c) an intracellular signaling domain, such that binding of the first and second CISC components to the CISC inducer molecule results in dimerization of the CISC components and a signal transduction event in the cell. Additionally, some nucleic acids and vectors provided herein comprise (1) a first nucleic acid sequence encoding a first chemically inducible signaling complex (CISC) component, and/or (2) a second nucleic acid sequence encoding a second chemically inducible signaling complex (CISC) component, each CISC component comprising (a) an extracellular binding domain that is capable of binding to a CISC inducer molecule, (b) a transmembrane domain, and (c) an intracellular signaling domain, such that binding of the first and second CISC components to the CISC inducer molecule results in dimerization of the CISC components and a signal transduction event in a cell.

Expression of CISC components in a cell allows selective induction of signaling in a cell by manipulation of the presence and/or concentration of the CISC inducer molecule. Such controllable induction of signaling allows, for example, selective expansion of cells expressing both CISC components, where the signal transduction event results in proliferation of the cell. In some embodiments, where two nucleic acids, each encoding a different CISC component, are introduced into the cell, such selective expansion allows for selection of cells that contain both nucleic acids, as contacting a cell comprising only one CISC component would not induce dimerization with the absent second CISC component.

Non-limiting examples of intracellular signaling domains include IL-2RB and IL-2RY intracellular domains and functional derivatives thereof. In some embodiments, an intracellular signaling domain of one CISC component comprises an IL-2Rγ intracellular domain or a functional derivative thereof, and an intracellular signaling domain of the other CISC component comprises an IL-2Rγ domain or a functional derivative thereof. In some embodiments, dimerization of the CISC components induces phosphorylation of JAK1, JAK3, and/or STAT5 in the cell. In some embodiments, dimerization of the CISC components induces proliferation of the cell.

Non-limiting examples of transmembrane domains include IL-2Rβ, IL-2Rγ, erythropoietin (Epo), and thrombopoietin (Tpo) transmembrane domains. In some embodiments, the transmembrane domain of a CISC component is derived from the same protein as the intracellular signaling domain of the CISC component (e.g., a CISC component comprising an IL-2Rβ intracellular domain comprises an IL-2Rβ transmembrane domain). In some embodiments, one CISC component comprises an IL-2Rβ transmembrane domain, and the other CISC component comprises an IL-2Rγ transmembrane domain.

Non-limiting examples of extracellular binding domains capable of binding a CISC inducer molecule include an FK506-binding protein (FKBP) domain and an FKBP-rapamycin-binding (FRB) domain. FKBP and FRB domains are capable of binding to rapamycin or rapalogs, such as those described below. In some embodiments, an extracellular binding domain of one CISC component comprises an FKBP domain, and an extracellular binding domain of the other CISC component comprises an FRB domain. In some embodiments, the CISC components form a heterodimer in the presence of the CISC inducer molecule.

Each of the extracellular binding domains, transmembrane domains, and intracellular signaling domains of the CISC components described herein may be connected to another domain of the same CISC component by a linker. Linkers are known in the art. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the glycine spacer comprises at least 3 glycines. In some embodiments, the glycine spacer comprises a sequence set forth as GGGS (SEQ ID NO: 26), GGGSGGG (SEQ ID NO: 27) or GGG.

An extracellular binding domain may be connected to a transmembrane domain by a hinge domain. A hinge refers to a domain that links the extracellular binding domain to the transmembrane domain, and may confer flexibility to the extracellular binding domain. In some embodiments, the hinge domain positions the extracellular domain close to the plasma membrane to minimize the potential for recognition by antibodies or binding fragments thereof. In some embodiments, the extracellular binding domain is located N-terminal to the hinge domain. In some embodiments, the hinge domain may be natural or synthetic.

In some embodiments, the CISC inducer molecule is rapamycin or a rapalog. In some embodiments, the CISC inducer molecule is rapamycin. Non-limiting examples of rapalogs include everolimus, CCl-779, C20-methallylrapamycin, C16-(S)-3-methylindolerapamycin, C16-iRap, C16-(S)-7-methylindolerapamycin, AP21967, C16(S)Butylsulfonamidorapamycin, AP23050, sodium mycophenolic acid, benidipine hydrochloride, AP1903, and AP23573, and metabolites or derivatives thereof.

In some embodiments, a method comprises introducing into a cell a nucleic acid encoding a third CISC component that is capable of binding to the CISC inducer molecule. Such CISC components are useful, for example, for binding to the intracellular CISC inducer molecules (e.g., intracellular rapamycin), thereby preventing the bound CISC inducer molecule from interacting with other intracellular molecules or structures (e.g., preventing rapamycin from interacting with mTOR). In some embodiments, the third CISC component is a soluble protein that does not comprise a transmembrane domain. In some embodiments, the third CISC component comprises an intracellular FRB domain. In some embodiments, a third CISC component is a soluble protein comprising an FRB domain and lacking a transmembrane domain.

Nucleic acids encoding a first, second, and/or third CISC component may be comprised in one or more vectors. In some embodiments, a nucleic acid encoding a first CISC component is present on a separate vector from a nucleic acid encoding the second CISC component. In some embodiments, a nucleic acid encoding the third CISC component is present on the same vector as a nucleic acid encoding the first or second CISC component. In other embodiments, a nucleic acid encoding the third CISC component is present on a distinct vector from nucleic acids encoding the first and/or second CISC components. In some embodiments, one or more vectors are viral vectors. In some embodiments, one or more vectors are lentiviral vectors. In some embodiments, one or more vectors are adeno-associated viral (AAV) vectors. In some embodiments, one or more AAV vectors is an AAV1, AAV2, AAV3, AAV4, AAV5, AAV6, AAV7, AAV8, AAV9, AAV10, or AAV11 vector. In some embodiments, one or more AAV vectors are AAV5 vectors. In some embodiments, one or more AAV vectors are AAV6 vectors.

In some embodiments, a CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 28 or 29. In some embodiments, one or more CISC components further comprise a signal peptide. The signal peptide may be any signal peptide known in the art that directs the translated CISC component to the cell membrane.

In some embodiments, one CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 28, and the other CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 29. In some embodiments, each CISC component further comprises a signal peptide, which may have the same or different amino acid sequences. The signal peptides may be any signal peptide known in the art that directs the translated CISC component to the cell membrane.

In some embodiments, a third CISC component comprises an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 30. In some embodiments, a third CISC component consists of an amino acid sequence with at least 80%, at least 90%, at least 91%, at least 92%, at least 93%, at least 94%, at least 95%, at least 96%, at least 97%, at least 98%, at least 99%, or up to 100% sequence identity to the amino acid sequence set forth as SEQ ID NO: 30. In some embodiments, the third CISC component does not comprise a signal peptide. In some embodiments, the third CISC component does not comprise a transmembrane domain.

Stabilized FoxP3 expression

Some embodiments of methods of modifying cells described herein comprise introducing a genetic modification in a cell that stabilizes expression of FoxP3. Similarly, some embodiments of cells described herein comprise a genetic modification that stabilizes or increases FoxP3expression, relative to an unmodified cell. Additionally, some embodiments of nucleic acids and vectors described herein stabilize FoxP3 expression in a cell.

In some embodiments, an endogenous FOXP3 locus is modified in a cell, resulting in stabilized expression. For example, in some embodiments, a heterologous promoter is inserted within or downstream from a Treg-specific demethylated region (TSDR) in the genome, and upstream from a first coding exon of an endogenous FOXP3 coding sequence. In some embodiments, a promoter is inserted downstream from the TSDR, and within or upstream from the first coding exon of FOXP3. Insertion of a heterologous promoter in this manner bypasses endogenous regulation of FOXP3 by the TSDR, which can become methylated in inflammatory conditions, inhibiting transcription of the endogenous FOXP3 coding sequence from the endogenous FOXP3 promoter located upstream from the TSDR. Thus, such stabilized FoxP3 expression by heterologous promoter insertion allows stable FoxP3 expression even in inflammatory conditions, preventing transdifferentiation into a T effector cell.

The heterologous promoter may be inserted at any position between the endogenous promoter and the first coding exon of the FOXP3 coding sequence. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides downstream from the TSDR of FOXP3. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides upstream from the first coding exon of the FOXP3 coding sequence. In some embodiments, the heterologous promoter is inserted into the first coding exon, such that a synthetic first coding exon is created, where the synthetic first coding exon differs from the endogenous first coding exon but still comprises a start codon that is in-frame with the FOXP3 coding sequence of downstream FOXP3 exons. In some embodiments, the heterologous promoter is inserted into the TSDR, such that the TSDR is modified and does not inhibit transcription of the endogenous FOXP3 coding sequence in inflammatory conditions.

In some embodiments, the nucleic acid comprising a heterologous promoter is comprised on a vector. In some embodiments, the vector is a viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAV9, AAV10, or AAV11 vector. In some embodiments, the AAV vector is an AAV5 vector. In some embodiments, the AAV vector is an AAV6 vector.

In some embodiments, a nucleic acid comprising a promoter operably linked to a nucleic acid sequence encoding FoxP3 or a functional derivative thereof is introduced into the cell. Expression of a heterologous promoter and sequence encoding FoxP3 is useful, for example, for expressing functional FoxP3 in cells containing genomic mutations in the FOXP3 coding sequence (e.g., cells from subjects having IPEX syndrome). Additionally, additional coding sequences (e.g., encoding a TGFβRI, Smad2, and/or Smad3 protein described herein) may be included in a nucleic acid, such that the heterologous promoter controls transcription of RNA encoding FoxP3 sequence and one or more other proteins (e.g., constitutively active TGFβRI, Smad2, and/or Smad3). In some embodiments, the sequence encoding FoxP3 is a cDNA sequence that does not comprise an intron.

The introduced nucleic acid may be integrated into the genome at a targeted locus (e.g., by homologous recombination), integrated in a non-targeted manner (e.g., by delivery on a lentiviral vector), or not integrated. In some embodiments, the nucleic acid comprises a 5′ homology arm that is upstream from the promoter, and a 3′ homology arm that is downstream from the nucleic acid sequence encoding FoxP3, and both homology arms have homology to a targeted locus in a genome. Such homology arms promote insertion of the nucleic acid into the genome at the targeted locus by homologous recombination. The homology arms may be the same length, have similar lengths (within 100 bp of each other), or different lengths. In some embodiments, one or both homology arms have a length of 200-2,000 bp, 400-1,500 bp, 500-1,000 bp. In some embodiments, one or both homology arms are about 100 bp, about 200 bp, about 300 bp, about 400 bp, about 500 bp, about 600 bp, about 700 bp, about 800 bp, about 900 bp, about 1,000 bp, about 1,100 bp, about 1,200 bp, about 1,300 bp, about 1,400 bp, about 1,500 bp, about 1,600 bp, about 1,700 bp, about 1,800 bp, about 1,900 bp, or about 2,000 bp.

In some embodiments, the nucleic acid is integrated at a FOXP3 locus in the genome. In some embodiments, the nucleic acid is integrated at a non-FOXP3 locus. In some embodiments, the targeted locus is a safe harbor locus. In some embodiments, the safe harbor locus is an AAVS1 locus, a HIPP11 locus, or a ROSA26 locus. In some embodiments, the nucleic acid is integrated at a TCRα (TRAC) locus. In some embodiments, the nucleic acid is integrated at a TCRβ (TRBC) locus.

In some embodiments, a nuclease capable of cleaving the genome at a targeted locus, or a nucleic acid encoding the nuclease (e.g., an mRNA) is introduced into the cell. Following delivery of the nuclease or transcription of the nuclease inside the cell, the nuclease introduces a double-stranded break at the targeted locus, thereby promoting integration of a donor template (e.g., nucleic acid comprising a promoter and sequence encoding FoxP3, or nucleic acid comprising a heterologous promoter for promoting transcription of an endogenous FOXP3 coding sequence) into the genome at the targeted locus by homology-directed repair. The nuclease may be any nuclease known in the art, including a meganuclease, zinc finger nuclease, TALEN, or RNA-guided nuclease. In embodiments where an RNA-guided nuclease (or nucleic acid encoding an RNA-guided nuclease) is delivered, a guide RNA (or nucleic acid encoding a guide RNA) comprising a spacer sequence complementary to a genomic sequence at the targeted locus is introduced into the cell. A gRNA or nucleic acid encoding a gRNA may be introduced into the cell with the nuclease or nucleic acid encoding the nuclease, or introduced separately (e.g., in a separate vector or delivery vehicle). The RNA-guided nuclease may be any RNA-guided nuclease known in the art or described herein in the section entitled “Nucleases.”

In some embodiments, a nucleic acid comprising a heterologous promoter operably linked to a sequence encoding FoxP3 or a functional derivative thereof is present on a vector. In some embodiments, the vector is a viral vector. In some embodiments, the vector is a lentiviral vector. In some embodiments, the vector is an adeno-associated virus (AAV) vector. In some embodiments, the AAV vector is an AAV1, AAV2, AAV3, AAV4, AAV5, AAV6, AAV7, AAV8, AAV9, AAV10, or AAV11 vector. In some embodiments, the AAV vector is an AAV5 vector. In some embodiments, the AAV vector is an AAV6 vector. In some embodiments, the vector is a plasmid. In some embodiments, the vector is bacterial artificial chromosome. In some embodiments, the vector is human artificial chromosome. In some embodiments, the vector integrates into a chromosome of the genome, and RNA encoding FoxP3 is transcribed from the genome of the cell. In other embodiments, the vector does not integrate into a chromosome, and the sequence encoding FoxP3 is expressed episomally.

The heterologous promoter inserted into the FOXP3 locus or operably linked to the FOXP3 coding sequence may be any promoter known in the art. In some embodiments, the promoter is a constitutive promoter. In some embodiments, the promoter is an MND, PGK, or EF-1a promoter. In some embodiments, the promoter is an MND promoter. In some embodiments, the promoter is an inducible promoter. In some embodiments, the promoter is inducible by a drug or steroid.

### Antigen-Specific Receptor Polypeptides

Some embodiments of methods for producing engineered cells described herein include introducing a nucleic acid encoding an antigen-specific receptor (e.g., T cell receptor (TCR) or chimeric antigen receptor (CAR)) polypeptide, or portion thereof (e.g., a TCRα chain or TCRβ chain), into a cell. Similarly, some embodiments of cells described herein comprise a nucleic acid encoding an antigen-specific receptor. Additionally, some embodiments of nucleic acids and vectors described herein (e.g., for in vivo administration) encode a TCR or CAR or portion thereof. In some embodiments, a nucleic acid is inserted into, or comprises homology arms for directing insertion into, a TRAC locus or TRBC locus.

In some embodiments, a nucleic acid is inserted into or designed for insertion into the TRAC or TRBC locus to capture the endogenous promoter. Promoter capture includes the introduction of an exogenous sequence into a locus such that its expression is driven by the endogenous promoter. For example, a cell may be edited (ex vivo or in vivo) by inserting a nucleic acid molecule comprising a nucleic acid encoding an exogenous TCR or CAR or portion thereof into the TRAC or TRBC locus, where the nucleic acid encoding the TCR or CAR is inserted downstream of (e.g., 11 to 10,000 bp downstream from) the endogenous TRAC or TRBC promoter, such that the endogenous TRAC or TRBC promoter becomes operably linked to the inserted nucleic acid and drives expression of the exogenous TCR or CAR.

In some embodiments, a nucleic acid is inserted into or designed for insertion into a TRAC or TRBC locus, such that insertion disrupts expression of the endogenous TCRα or TCRβ chain. In some embodiments, the coding sequence of the endogenous TCRα or TCRβ chain, or a portion of the coding sequence, is removed from the locus such that the endogenous TCRα or TCRβ is not expressed in the cell. In some embodiments, the inserted nucleic acid comprises a heterologous promoter that drives expression of the inserted TCR or CAR.

In some embodiments, a nucleic acid is inserted into or designed for insertion into a TRAC or TRBC locus to hijack the endogenous TRAC or TRBC gene with a heterologous promoter. For example, a cell may be edited by inserting a polynucleotide molecule comprising a promoter operably linked to (a) a nucleic acid encoding a full-length TCRβ protein, and to a nucleic acid encoding TCRα variable (TRAV) and TCR joining (TRAJ) regions, where the coding sequences of the TRAV and TRAJ regions are inserted in-frame with the coding sequences encoding the TCRα constant regions, such that the inserted heterologous promoter controls transcription of a heterologous TCRβ protein and transcription of a TCRα protein comprising heterologous TRAV/TRAJ amino acid sequences and an endogenous TCRα constant region amino acid sequence. This embodiment utilizes the endogenous 3′ regulatory region from the endogenous TRAC gene. A similar approach may be used to hijack the endogenous TRBC locus, where the encoded full-length protein is a TCRα chain, and the nucleic acid further encodes TCRβ variable and TCRβ joining regions in-frame with TCRβ constant regions.

In some embodiments, an antigen-specific receptor is expressed episomally in a cell. Episomal expression may be achieved by any method known in the art, such as delivery of an RNA (e.g., mRNA or self-amplifying RNA) or DNA (e.g., plasmid or artificial chromosome) encoding the antigen-specific receptor.

In embodiments comprising use of a heterologous promoter to express a TCR, CAR, or portion thereof, the heterologous promoter may be any promoter known in the art. In some embodiments, the heterologous promoter is a constitutive promoter. In some embodiments, the promoter is an MND promoter.

TGFβRII and/or Smad4 Expression

Some embodiments of the methods of modifying cells described herein comprise introducing into the cell one or more nucleic acids that encode TGFβRII and/or Smad4. Similarly, some embodiments of cells described herein comprise one or more nucleic acid sequences encoding TGFβRII and/or Smad4. Additionally, some nucleic acids and vectors described herein further comprise a nucleic acid sequence encoding TGFβRII and/or a nucleic acid sequence encoding Smad4.

As described above, TGF-β signals through a cooperative interaction between two transmembrane receptors, TGFβRI and TGFβRII. Binding of TGF-β to TGFβRII allows recruitment of the TGFβRI, after which TGFβRII phosphorylates TGFβRI at a juxtamembrane glycine-serine repeat (e.g., TTSGSGSG of SEQ ID NO: 23). Downstream signal transduction may then occur by phosphorylation of Smad2 and/or Smad3, e.g., at a C-terminal Ser-Ser-X-Ser motif. Two monomers of activated Smad2 or Smad3 (e.g., phosphorylated Smad2/Smad3 or mutant forms containing phosphomimetic amino acids (e.g., aspartate and/or glutamate)) may then complex with a monomer of common Smad, Smad4, to form a heterotrimer comprising one Smad4 subunit and two subunits of Smad2 and/or SMAD3 (e.g., two Smad2, two Smad3, or a Smad2 and a Smad3 subunit). This heterotrimer may effect changes in gene expression directly by binding to Smad-binding elements (SBEs) in TGF-β-regulated genes and/or indirectly by interacting with other transcription factors.

Accordingly, in some embodiments, a method described herein further comprises introducing a nucleic acid sequence encoding TGFβRII. Such a nucleic acid sequence may be present on another nucleic acid or vector introduced into the cell (e.g., a nucleic acid or vector encoding TGFβRI), or a different nucleic acid or vector. In some embodiments, a modified cell described herein further comprises a nucleic acid sequence encoding TGFβRII. Such a nucleic acid sequence may be present at any locus described herein (e.g., a safe harbor locus, or a TGFβRI locus), a different locus, or episomally on an introduced nucleic acid or vector. In some embodiments, a nucleic acid described herein further comprises a nucleic acid sequence encoding TGFβRII. In some embodiments, a vector described herein further comprises a nucleic acid sequence encoding TGFβRII. Nucleic acid sequences encoding TGFβRII are known in the art, including the coding sequence of human TGFβRII set forth in nucleotides 284-1984 of GenBank Accession No. NM_003242.6, Nucleic acid sequences encoding TGFβRII may comprise one or more substitutions that do not change the amino acid sequence of the encoded TGFβRII. In some embodiments, the nucleic acid sequence is codon-optimized for expression in a cell. In some embodiments, the nucleic acid sequence is codon-optimized for expression in a human cell. Amino acid sequences of TGFβRII are also known in the art, including the amino acid sequence of human TGFβRII set forth in UniProt Accession No. A3QNQ0. An amino acid sequence of TGFβRII encoded by a nucleic acid sequence described herein may comprise one or more conservative amino acid substitutions, relative to the amino acid sequence set forth in UniProt Accession No. A3QNQ0.

Similarly, in some embodiments, a method described herein further comprises introducing a nucleic acid sequence encoding Smad4. Such a nucleic acid sequence may be present on another nucleic acid or vector introduced into the cell (e.g., a nucleic acid or vector encoding Smad2 or Smad3), or a different nucleic acid or vector. In some embodiments, a modified cell described herein further comprises a nucleic acid sequence encoding Smad4. Such a nucleic acid sequence may be present at any locus described herein (e.g., a safe harbor locus, SMAD2 locus, or SMAD3 locus), a different locus, or episomally on an introduced nucleic acid or vector. In some embodiments, a nucleic acid described herein further comprises a nucleic acid sequence encoding Smad4. In some embodiments, a vector described herein further comprises a nucleic acid sequence encoding Smad4. Nucleic acid sequences encoding Smad4 are known in the art, including the coding sequence of human

Smad4 set forth in nucleotides 539-2194 of GenBank Accession No. NM_005359.6, Nucleic acid sequences encoding Smad4 may comprise one or more substitutions that do not change the amino acid sequence of the encoded Smad4. In some embodiments, the nucleic acid sequence is codon-optimized for expression in a cell. In some embodiments, the nucleic acid sequence is codon-optimized for expression in a human cell. Amino acid sequences of Smad4 are also known in the art, including the amino acid sequence of human Smad4 set forth in UniProt Accession No. Q13485. An amino acid sequence of Smad4 encoded by a nucleic acid sequence described herein may comprise one or more conservative amino acid substitutions, relative to the amino acid sequence set forth in UniProt Accession No. Q13485.

### Compositions for Producing Engineered Cells

Some aspects of the disclosure relate to compositions for engineering Tregs by upregulating the TGF-β pathway in cells either in vivo, in vitro, or ex vivo.

Some aspects of the disclosure relate to compositions for administering into a subject so that they target particular cells (e.g., immune cells or pluripotent cells) and upregulate TGF-β signaling so as to be engineered into Treg cells in vivo. Such compositions may include nucleic acids, e.g., comprised in vectors (e.g., viral or non-viral vectors) or formulated using nanoparticles, that encode constitutively active positive regulators of the TGF-β pathway or inhibitors of negative regulators of the TGF-β pathway. In some embodiments, such compositions include nucleic acids encoding constitutively active members of the TGF-β pathway that positively regulate the pathway. In some embodiments, compositions for making Tregs according to the methods disclosed herein comprise inhibitors or members of the TGF-β pathway that negatively regulate the pathway. In some embodiments, compositions for making Tregs according to the methods disclosed herein comprises a nucleic acid that induces or increases expression of constitutively active Smad2. In some embodiments, compositions for making Tregs according to the methods disclosed herein comprises a nucleic acid that induces or increases expression of constitutively active SMAD3. In some embodiments, compositions for making Tregs according to the methods disclosed herein comprises a nucleic acid that induces or increases expression of constitutively active Smad2 and Smad3. In some embodiments, compositions for making Tregs according to the methods disclosed herein comprises a nucleic acid that decreases expression of Smad7. In some embodiments, compositions for making Tregs according to the methods disclosed herein comprises a nucleic acid that induces or increases expression of constitutively active Smad2 and/or Smad3 and nucleic acid that decreases expression of Smad7.

In some embodiments, a composition for making Tregs according to the methods disclosed herein comprises one or more nucleic acids encoding T cell receptors (TCRs), chimeric antigen receptors (CARs) to target particular cells, enhance suppressive function, or both. In some embodiments, a composition comprises a nucleic acid encoding IL-2. In some embodiments, a composition for making Tregs according to the methods disclosed herein comprises a nucleic acid encoding a constitutively active IL-10 (see, e.g., WO 2019/180724, which is incorporated herein by reference in its entirety), CISC components, and/or a soluble FRB protein untethered from mTOR (see, e.g., WO 2018/111834, WO 2019/210057, and WO 2020/264039, each of which is incorporated herein by reference in its entirety).

In some embodiments, a nucleic acid described herein (e.g., for introduction into a cell or administration to a subject) is comprised in a vector. The term “vector” is used to refer to any molecule (e.g., nucleic acid, plasmid) or arrangement of molecules (e.g., virus) used to transfer coding information to a host cell. The term “expression vector” refers to a vector that is suitable for introduction of a host cell and contains nucleic acid sequences that direct and/or control expression of introduced heterologous nucleic acid sequences. Expression includes, but is not limited to, processes such as transcription, translation, and RNA splicing, if introns are present. Non-limiting examples of vectors include artificial chromosomes, minigenes, cosmids, plasmids, phagemids, and viral vectors. Non-limiting examples of viral vectors include lentiviral vectors, retroviral vectors, herpesvirus vectors, adenovirus vectors, and adeno-associated viral vectors. In some embodiments, one or more vectors comprising nucleic acids for use in the methods provided herein are lentiviral vectors. In some embodiments, one or more vectors are adenoviral vectors. In some embodiments, one or more vectors are adeno-associated viral (AAV) vectors. In some embodiments, one or more AAV vectors is an AAV1, AAV2, AAV3, AAV4, AAV5, AAV6, AAV7, AAV8, AAV9, AAV10, or AAV11 vector. In some embodiments, one or more AAV vectors are AAV5 vectors. In some embodiments, one or more AAV vectors are AAV6 vectors.

As will be understood by those skilled in the art, nucleic acids may include genomic sequences, extra-genomic and plasmid-encoded sequences and smaller engineered gene segments that express, or may be adapted to express, proteins, polypeptides, peptides and the like. Such segments may be naturally isolated or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence encoding a variant or derivative of such a sequence.

In some embodiments, polynucleotide variants may have substantial identity to a reference polynucleotide sequence encoding an immunomodulatory polypeptide described herein. For example, a polynucleotide may be a polynucleotide comprising at least 70% sequence identity, preferably at least 75%, 80%, 85%, 90%, 95%, 96%, 97%, 98%, or 99% or higher, sequence identity or a sequence identity that is within a range defined by any two of the aforementioned percentages as compared to a reference polynucleotide sequence such as a sequence encoding an antibody described herein, using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the binding affinity of a polypeptide variant of a given polypeptide which is capable of a specific binding interaction with another molecule and is encoded by the variant polynucleotide is not substantially diminished relative to a polypeptide encoded by a polynucleotide sequence specifically set forth herein.

Some embodiments of nucleic acid sequences described herein (e.g., sequences on nucleic acids, vectors, or in cells) are codon-optimized for expression in a cell. The terms “codon-optimized” and “codon optimization,” with respect to a gene or coding sequence present in or introduced into a host cell, refer to alteration of codons in the gene or coding sequence to reflect the typical codon usage of the host cell, without altering the amino acid sequence encoded by the gene or coding sequence. Such optimization includes replacing at least one, or more than one, or a significant number, of codons with one or more codons that are more frequently used in the genes of that organism. Codon usage tables are readily available, for example, at the “Codon Usage Database” available at www.kazusa.or.jp. By utilizing the knowledge on codon usage or codon preference in each organism, one of ordinary skill in the art can apply the frequencies to any polypeptide with a given amino acid sequence, to produce a codon-optimized coding sequence which encodes the same polypeptide having the same amino acid sequence, but uses codons optimal for a given species (e.g., a human). Codon-optimized coding regions can be designed by various methods known to those skilled in the art.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the case of preparation and use in the intended recombinant DNA protocol. For example, illustrative polynucleotide segments with total lengths of or about of 10,000, 5000, 3000, 2,000, 1,000, 500, 200,100, or 50 base pairs in length, and the like, (including all intermediate lengths) are contemplated to be useful.

When comparing polynucleotide or nucleic acid sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least or at least about 20 contiguous positions, usually 30 to 75, or 40 to 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins-Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., Unified Approach to Alignment and Phylogenes, pp. 626-645 (1990); Methods in Enzymology vol. 183, Academic Press, Inc., San Diego, CA; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Santou, N. Nes, M., Mol. Biol. Evol. 4:406-425 (1987); Sncath, P. H. A. and Sokal, R. R., Numerical Taxonomy—the Principles and Practice of Numerical Taxonomy, Freeman Press, San Francisco, CA (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85:2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One preferred example of algorithms that are suitable for determining percent sequence identity and sequence similarity are the BLAST and BLAST 2.0 algorithms, which are described in Altschul et al., Nucl Acids Res. 1977. 25:3389-3402, and Altschul et al., J Mol Biol. 1990. 215:403-410, respectively. BLAST and BLAST 2.0 can be used, for example with the parameters described herein, to determine percent sequence identity among two or more the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W. T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff, Proc Natl Acad Sci USA. 1989. 89:10915) alignments, (B) of 50, expectation (E) of 10, M=5, N=−4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

### Pharmaceutical Compositions

Some aspects of the disclosure relate to a pharmaceutical composition comprising a cell, vector, or nucleic acid described herein, and a pharmaceutically acceptable excipient or carrier. Such pharmaceutical compositions are formulated, for example, for systemic administration, or administration to target tissues. “Acceptable” means that the excipient (carrier) must be compatible with the active ingredient of the composition (and preferably, capable of stabilizing the active ingredient) and not deleterious to the subject to be treated. Pharmaceutically acceptable excipients, carriers, buffers, stabilizers, isotonicizing agents, preservatives or antioxidants, or other materials well known to those skilled in the art. Such materials should be non-toxic and should not interfere with the efficacy of the active ingredient. The precise nature of the carrier or other material may depend on the route of administration, e.g., parenteral, intramuscular, intradermal, sublingual, buccal, ocular, intranasal, subcutaneous, intrathecal, intratumoral, oral, vaginal, or rectal. Scc, e.g., Remington: The Science and Practice of Pharmacy 20th Ed. (2000) Lippincott Williams and Wilkins, Ed. K. E. Hoover. The pharmaceutical compositions to be used for in vivo administration must be sterile, with the exception of any cells, viruses, and/or viral vectors being used to achieve a biological effect (e.g., immunosuppression). This is readily accomplished by, for example, filtration through sterile filtration membranes. The pharmaceutical compositions described herein may be placed into a container having a sterile access port, for example, an intravenous solution bag or vial having a stopper pierceable by a hypodermic injection needle.

In some embodiments, the pharmaceutical compositions described herein can be formulated for intramuscular injection, intravenous injection, intradermal injection, or subcutaneous injection.

The pharmaceutical compositions described herein to be used in the present methods can comprise pharmaceutically acceptable carriers, buffer agents, excipients, salts, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, the pharmaceutical composition described herein comprises lipid nanoparticles which can be prepared by methods known in the art, such as described in Epstein et al., Proc Natl Acad Sci USA.1985. 82:3688; Hwang et al. Proc Natl Acad Sci USA. 1980. 77:4030; and U.S. Pat. Nos. 4,485,045 and 4,544,545. Liposomes with enhanced circulation time are disclosed in U.S. Pat. No. 5,013,556, Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Lipids used in the formulation of lipid nanoparticles for delivering nucleic acids are generally known in the art, and include ionizable amino lipids, non-cationic lipids, sterols, and polyethylene glycol-modified lipids. Sec, e.g., Buschmann et al., Vaccines. 2021. 9 (1): 65. In some embodiments, the nucleic acid is surrounded by the lipids of the lipid nanoparticle and present in the interior of the lipid nanoparticle. In some embodiments, the nucleic acid is dispersed throughout the lipids of the lipid nanoparticle. In some embodiments, the lipid nanoparticle comprises an ionizable amino lipid, a non-cationic lipid, a sterol, and/or a polyethylene glycol (PEG)-modified lipid.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

Pharmaceutical compositions described herein may be useful for treating a subject that has or is at risk of developing an inflammatory, autoimmune, or allergic condition or disease. A subject having or at risk of developing an inflammatory, autoimmune, or allergic condition or disease may be identified by ascertaining the presence and/or absence of one or more risk factors, diagnostic indicators, or prognostic indications. The determination may be made based on clinical, cellular, or serologic findings, including flow cytometry, serology, and/or DNA analyses known in the art.

The pharmaceutical compositions described herein can include a therapeutically effective amount of any cell, vector, and/or nucleic acid described herein. For example, in some embodiments, the pharmaceutical composition includes a cell, vector, or nucleic acid at any of the doses described herein.

A “therapeutically effective amount” refers to an amount effective, at dosages and for periods of time necessary, to achieve a desired therapeutic result. The therapeutically effective amount may vary according to factors such as the age, sex, and weight of the individual, and the ability of the cell, nucleic acid, or vector to effect a desired response in the subject.

Pharmaceutical compositions can be prepared in accordance with known techniques. See, e.g., Remington, The Science And Practice of Pharmacy (21st ed., Philadelphia, Lippincott, Williams & Wilkins, 2005). For example, cells, vectors, or nucleic acids described herein may be admixed with a pharmaceutically acceptable excipient, and the resulting composition is administered to a subject. The carrier must be acceptable in the sense of being compatible with any other ingredients in the formulation and must not be deleterious to the subject. The carrier can be a solid or a liquid, or both, and can be formulated with the compound as a unit-dose formulation.

In some embodiments, a pharmaceutical composition comprises cells at a dose of about 10⁴ to about 10¹⁰ cells/kg. In some embodiments, the pharmaceutical composition comprises cells at a dose of about: 10⁴ to 10⁵, 10⁵ to 10⁶, 10⁶ to 10⁷, 10⁷ to 10⁸, 10⁸ to 10⁹, or 10⁹ to 10¹⁰ cells/kg. In some embodiments, a pharmaceutical composition comprises cells at a dose of about 0.1×10⁶, 0.2×10⁶, 0.3×10⁶, 0.4×10⁶, 0.5×10⁶, 0.6×10⁶, 0.7×10⁶,0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×106, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×106, 3.6×10⁶, 3.7×10⁶, 3.8×10⁶, 3.9×10⁶, 4.0×10⁶, 4.1×10⁶, 4.2×10⁶, 4.3×10⁶, 4.4×106, 4.5×10⁶, 4.6×10⁶, 4.7×10⁶, 4.8×10⁶, 4.9×10⁶, 5.0×10⁶, 5.1×10⁶, 5.2×10⁶, 5.3×106, 5.4×10⁶, 5.5×10⁶, 5.6×10⁶, 5.7×10⁶, 5.8×10⁶, 5.9×10⁶, 6.0×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7.0×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8.0×10⁶, 8.1×10⁶, 8.2×10⁶, 8.3×10⁶, 8.4×10⁶, 8.5×10⁶, 8.6×10⁶, 8.7×106, 8.8×10⁶,8.9×10⁶, 9.0×10⁶, 9.1×10⁶, 9.2×10⁶, 9.3×10⁶,9.4×10⁶, 9.5×10⁶, 9.6×10⁶, 9.7×10⁶, 9.8×10⁶, 9.9×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.1×10⁷, 2.2×10⁷, 2.3×10⁷, 2.4×10⁷, 2.5×10⁷, 2.6×10⁷, 2.7×10⁷, 2.8×10⁷, 2.9×10⁷, 3.0×10⁷, 3.1×10⁷, 3.2×10⁷, 3.3×10⁷, 3.4×10⁷, 3.5×10⁷, 3.6×10⁷, 3.7×10⁷, 3.8×10⁷, 3.9×10⁷, 4.0×10⁷, 4.1×10⁷, 4.2×10⁷, 4.3×10⁷, 4.4×10⁷, 4.5×10⁷, 4.6×10⁷, 4.7×10⁷, 4.8×10⁷, 4.9×10⁷, 5.0×10⁷, 5.1×10⁷, 5.2×10⁷, 5.3×10⁷, 5.4×10⁷, 5.5×10⁷, 5.6×10⁷, 5.7×10⁷, 5.8×10⁷, 5.9×10⁷, 6.0×10⁷, 6.1×10⁷, 6.2×10⁷, 6.3×10⁷, 6.4×10⁷, 6.5×10⁷, 6.6×10⁷, 6.7×10⁷, 6.8×10⁷, 6.9×10⁷, 7.0×10⁷, 7.1×10⁷, 7.2×10⁷, 7.3×10⁷, 7.4×10⁷, 7.5×10⁷, 7.6×10⁷, 7.7×10⁷, 7.8×10⁷, 7.9×10⁷, 8.0×10⁷, 8.1×10⁷, 8.2×10⁷, 8.3×10⁷, 8.4×10⁷, 8.5×10⁷, 8.6×10⁷, 8.7×10⁷, 8.8×10⁷, 8.9×10⁷, 9.0×10⁷, 9.1×10⁷, 9.2×10⁷, 9.3×10⁷, 9.4×10⁷, 9.5×10⁷, 9.6×10⁷, 9.7×10⁷, 9.8×10⁷, 9.9×10⁷, or 1.0×10⁸ cells/kg.

In some embodiments, a pharmaceutical composition comprises an effective amount of a vector or nucleic acid described herein. In some examples, the pharmaceutical composition comprises about 0.1 mg/kg to about 3 mg/kg of the vector or nucleic acid. In some embodiments, the pharmaceutical composition comprises about 0.1 mg/kg, about 0.25 mg/kg, about 0.5 mg/kg, about 0.75 mg/kg, about 1.0 mg/kg, about 1.5 mg/kg, about 2.0 mg/kg, about 2.5 mg/kg, or about 3.0 mg/kg of the vector or nucleic acid. In some embodiments, pharmaceutical composition comprises about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg of the vector or nucleic acid.

In some embodiments, the pharmaceutical composition comprises a vector or nucleic acid encapsulated within a lipid nanoparticle.

Some embodiments of lipid nanoparticles comprise at least one cationic lipid, at least one non-cationic lipid, and at least one conjugated lipid. In more particular examples, lipid nanoparticles can comprise from about 50 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology. In other particular examples, lipid nanoparticles can comprise from about 40 mol % to about 85 mol % of a cationic lipid, from about 13 mol % to about 49.5 mol % of a non-cationic lipid, and from about 0.5 mol % to about 10 mol % of a lipid conjugate and are produced in such a manner as to have a non-lamellar (i.e., non-bilayer) morphology.

Cationic lipids can include, for example, one or more of the following: palmitoyi-oleoyl-nor-arginine (PONA), MPDACA, GUADACA, ((6Z,9Z,28Z,31Z)-heptatriaconta-6,9,28,31-tetraen-19-yl 4-(dimethylamino) butanoate) (MC3), LenMC3, CP-LenMC3, γ-LenMC3, CP-γ-LenMC3, MC3MC, MC2MC, MC3 Ether, MC4 Ether, MC3 Amide, Pan-MC3, Pan-MC4 and Pan MC5, 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminocthyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linolcyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino) ethoxypropane (DLin-EG-DMA), N,N-diolcyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy) propyl)-N,N,N-trimethylammonium chloride (DOTMA), N,N-distearyl-N,N-dimethylammonium bromide (DDAB), N-(1-(2,3-diolcoyloxy) propyl)-N,N,N-trimethylammonium chloride (DOTAP), 3-(N—(N′,N′-dimethylaminoethane)-carbamoyl) cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), or mixtures thereof. The cationic lipid can also be DLinDMA, DLin-K-C2-DMA (“XTC2”), MC3, LenMC3, CP-LenMC3, γ-LenMC3, CP-γ-LenMC3, MC3MC, MC2MC, MC3 Ether, MC4 Ether, MC3 Amide, Pan-MC3, Pan-MC4, Pan MC5, or mixtures thereof.

In various embodiments, the cationic lipid may comprise from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, or from about 50 mol % to about 60 mol % of the total lipid present in the particle.

In other embodiments, the cationic lipid may comprise from about 40 mol % to about 90 mol %, from about 40 mol % to about 85 mol %, from about 40 mol % to about 80 mol %, from about 40 mol % to about 75 mol %, from about 40 mol % to about 70 mol %, from about 40 mol % to about 65 mol %, or from about 40 mol % to about 60 mol % of the total lipid present in the particle.

The non-cationic lipid may comprise, e.g., one or more anionic lipids and/or neutral lipids. In particular embodiments, the non-cationic lipid comprises one of the following neutral lipid components: (1) cholesterol or a derivative thereof; (2) a phospholipid; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The phospholipid may be a neutral lipid including, but not limited to, dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain embodiments, the phospholipid is DPPC, DSPC, or mixtures thereof.

In some embodiments, the non-cationic lipid (e.g., one or more phospholipids and/or cholesterol) may comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle. When the non-cationic lipid is a mixture of a phospholipid and cholesterol or a cholesterol derivative, the mixture may comprise up to about 40, 50, or 60 mol % of the total lipid present in the particle.

The conjugated lipid that inhibits aggregation of particles may comprise, e.g., one or more of the following: a polyethyleneglycol (PEG)-lipid conjugate, a polyamide (ATTA)-lipid conjugate, a cationic-polymer-lipid conjugates (CPLs), or mixtures thereof. In one particular embodiment, the nucleic acid-lipid particles comprise either a PEG-lipid conjugate or an ATTA-lipid conjugate. In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEG-DAA conjugate may be PEG-di lauryloxypropyl (C12), a PEG-dimyristyloxypropyl (C14), a PEG-dipalmityloxypropyl (C16), a PEG-distearyloxypropyl (C18), or mixtures thereof.

Additional PEG-lipid conjugates suitable for use in the invention include, but are not limited to, mPEG2000-1,2-di-O-alkyl-sn3-carbomoylglyceride (PEG-C-DOMG). The synthesis of PEG-C-DOMG is described in PCT Application No. PCT/US08/88676, Yet additional PEG-lipid conjugates suitable for use in the invention include, without limitation, 1-[8′-(1,2-dimyristoyl-3-propanoxy)-carboxamido-3′,6′-dioxaoctanyl]carbamoyl-ω-methyl-poly(ethylene glycol) (2KPEG-DMG). The synthesis of 2KPEG-DMG is described in U.S. Pat. No. 7,404,969.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

In other embodiments, the composition may comprise amphoteric liposomes, which contain at least one positive and at least one negative charge carrier, which differs from the positive one, the isoelectric point of the liposomes being between 4 and 8. This objective is accomplished owing to the fact that liposomes are prepared with a pH-dependent, changing charge.

Liposomal structures with the desired properties are formed, for example, when the amount of membrane-forming or membrane-based cationic charge carriers exceeds that of the anionic charge carriers at a low pH and the ratio is reversed at a higher pH. This is always the case when the ionizable components have a pKa value between 4 and 9. As the pH of the medium drops, all cationic charge carriers are more charged and all anionic charge carriers lose their charge.

Cationic compounds useful for amphoteric liposomes include those cationic compounds previously described herein above. Without limitation, strongly cationic compounds can include, for example: DC-Chol 3-B—[N—(N′,N′-dimethylmethane) carbamoyl]cholesterol, TC-Chol 3-β—[N—(N′, N′, N′-trimethylaminoethane) carbamoyl cholesterol, BGSC bisguanidinium-spermidine-cholesterol, BGTC bis-guadinium-tren-cholesterol, DOTAP (1,2-diolcoyloxypropyl)-N,N,N-trimethylammonium chloride, DOSPER (1,3-diolcoyloxy-2-(6-carboxy-spermyl)-propylarnide, DOTMA (1,2-diolcoyloxypropyl)-N,N,N-trimethylamronium chloride) (Lipofectin®), DORIE 1,2-diolcoyloxypropyl)-3-dimethylhydroxyethylammonium bromide, DOSC (1,2-dioleoyl-3-succinyl-sn-glyceryl choline ester), DOGSDSO (1,2-dioleoyl-sn-glycero-3-succinyl-2-hydroxyethyl disulfide omithine), DDAB dimethyldioctadecylammonium bromide, DOGS ((C18) 2GlySper3+) N,N-dioctadecylamido-glycol-spermin (Transfectam®) (C18) 2Gly+N,N-dioctadecylamido-glycine, CTAB cetyltrimethylarnmonium bromide, CpyC cetylpyridinium chloride, DOEPC 1,2-diolcoly-sn-glycero-3-ethylphosphocholine or other O-alkyl-phosphatidylcholine or ethanolamines, amides from lysine, arginine or ornithine and phosphatidyl ethanolamine.

Examples of weakly cationic compounds include, without limitation: His-Chol (histaminyl-cholesterol hemisuccinate), Mo-Chol (morpholine-N-ethylamino-cholesterol hemisuccinate), or histidinyl-PE.

Examples of neutral compounds include, without limitation: cholesterol, ceramides, phosphatidyl cholines, phosphatidyl ethanolamines, tetraether lipids, or diacyl glycerols.

Anionic compounds useful for amphoteric liposomes include those non-cationic compounds previously described herein. Without limitation, examples of weakly anionic compounds can include: CHEMS (cholesterol hemisuccinate), alkyl carboxylic acids with 8 to 25 carbon atoms, or diacyl glycerol hemisuccinate. Additional weakly anionic compounds can include the amides of aspartic acid, or glutamic acid and PE as well as PS and its amides with glycine, alanine, glutamine, asparagine, serine, cysteine, threonine, tyrosine, glutamic acid, aspartic acid or other amino acids or aminodicarboxylic acids. According to the same principle, the esters of hydroxycarboxylic acids or hydroxydicarboxylic acids and PS are also weakly anionic compounds.

In some embodiments, amphoteric liposomes may contain a conjugated lipid, such as those described herein above. Particular examples of useful conjugated lipids include, without limitation, PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Particular examples are PEG-modified diacylglycerols and dialkylglycerols.

In some embodiments, the neutral lipids may comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

Considering the total amount of neutral and conjugated lipids, the remaining balance of the amphoteric liposome can comprise a mixture of cationic compounds and anionic compounds formulated at various ratios. The ratio of cationic to anionic lipid may selected in order to achieve the desired properties of nucleic acid encapsulation, zeta potential, pKa, or other physicochemical property that is at least in part dependent on the presence of charged lipid components.

In some embodiments, the lipid nanoparticles have a composition that specifically enhances delivery and uptake in stem cells, hematopoietic cells, or T cells.

In some embodiments, the pharmaceutical composition comprises an effective amount of a lipid nanoparticle formulation, wherein the lipid nanoparticle formulation comprises a vector or nucleic acid described herein. In some examples, the lipid nanoparticle formulation comprises about 0.1 mg/kg to about 3 mg/kg of the vector or nucleic acid. In some embodiments, the lipid nanoparticle formulation comprises about 0.1 mg/kg, about 0.25 mg/kg, about 0.5 mg/kg, about 0.75 mg/kg, about 1.0 mg/kg, about 1.5 mg/kg, about 2.0 mg/kg, about 2.5 mg/kg, or about 3.0 mg/kg of the vector or nucleic acid. In some embodiments, the lipid nanoparticle formulation comprises about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg of the vector or nucleic acid.

In some embodiments, the pharmaceutical composition comprises an effective amount of a lipid nanoparticle formulation comprising a donor template comprising a template nucleic acid described herein, wherein lipid nanoparticle formulation comprises about 0.1 mg/kg to about 3 mg/kg of the donor polynucleotide. In some embodiments, the lipid nanoparticle formulation comprises about 0.1 mg/kg, about 0.25 mg/kg, about 0.5 mg/kg, about 0.75 mg/kg, about 1.0 mg/kg, about 1.5 mg/kg, about 2.0 mg/kg, about 2.5 mg/kg, or about 3.0 mg/kg of the donor polynucleotide. In some embodiments, the lipid nanoparticle formulation comprises about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg of the donor polynucleotide.

In some embodiments, pharmaceutical compositions of the invention can further comprise one or more additional agents useful in the treatment of an inflammatory, autoimmune, or allergic condition or disease in a subject.

Methods of use

Some aspects of the disclosure relate to methods of administering a cell, vector, nucleic acid, or lipid nanoparticle described herein to a subject. In some embodiments, a method comprises administering to a subject any one of the cells described herein. In some embodiments, a method comprises administering to the subject a cell that had previously been obtained from that subject before being administered (i.e., the cell is an autologous cell). In some embodiments, a method comprises (i) isolation of cells from a subject; (ii) processing the cells by any method (e.g., gene editing or introducing a vector) described herein; and (iii) administering the processed cells to the same subject. In some embodiments, a method comprises administering to the subject a cell that had previously been obtained from a different subject than the one to whom the cell is administered (i.e., the cell is an allogeneic cell). In some embodiments, a method comprises (i) isolation of cells from a first subject; (ii) processing the cells by any method (e.g., gene editing or introducing a vector) described herein; and (iii) administering the processed cells to a second subject.

Some embodiments of the methods, cells, systems, and compositions described herein include any of the cells, vectors, nucleic acids, or lipid nanoparticles described herein, for use as a medicament. In some embodiments, the cell, vector, nucleic acid, or lipid nanoparticle is for use in a method of preventing, treating, inhibiting, or ameliorating an inflammatory, autoimmune, or allergic condition or disease in a subject.

In some embodiments, a cell is described herein for use in a method of preventing, treating, inhibiting, or ameliorating an inflammatory, autoimmune, or allergic condition or disease in a subject. In some embodiments, the cell is autologous to the subject (i.e., derived from the subject). In other embodiments, the cell is allogeneic to the subject (i.e., derived from a different subject).

Inflammatory disease

In some embodiments, a cell, vector, nucleic acid, or lipid nanoparticle is used for treating, preventing, treating, inhibiting, or ameliorating an inflammatory condition or disease in a subject. In some embodiments, the subject has or is at risk of developing an inflammatory condition or disease. In some embodiments, the inflammatory condition or disease is selected from pancreatic islet cell transplantation, asthma, hepatitis, traumatic brain injury, primary sclerosing cholangitis, primary biliary cholangitis, polymyositis, stroke, Still's disease, acute respiratory distress syndrome (ARDS), uveitis, inflammatory bowel disease (IBD), ulcerative colitis, graft-versus-host disease (GvHD), tolerance induction for transplantation, transplant rejection, and sepsis.

In some embodiments, the inflammatory condition is associated with pancreatic islet cell transplantation. In some embodiments, the inflammatory disease is asthma. In some embodiments, the inflammatory disease is hepatitis. In some embodiments, the inflammatory condition is traumatic brain injury. In some embodiments, the inflammatory disease is primary sclerosing cholangitis. In some embodiments, the inflammatory disease is primary biliary cholangitis. In some embodiments, the inflammatory disease is polymyositis. In some embodiments, the inflammatory condition is stroke. In some embodiments, the inflammatory disease is Still's disease. In some embodiments, the inflammatory disease is acute respiratory distress syndrome (ARDS). In some embodiments, the inflammatory disease is uveitis. In some embodiments, the inflammatory disease is inflammatory bowel disease (IBD). In some embodiments, the inflammatory disease is graft-versus-host disease (GvHD). In some embodiments, the inflammatory condition is tolerance induction for transplantation. In some embodiments, the inflammatory condition is transplant rejection. In some embodiments, the inflammatory disease is sepsis.

In some embodiments, the cell expresses an antigen-specific receptor (e.g., T cell receptor or chimeric antigen receptor) that is specific to an antigen associated with the inflammatory condition or disease.

Autoimmune diseases

In some embodiments, a cell, vector, nucleic acid, or lipid nanoparticle is used for treating, preventing, treating, inhibiting, or ameliorating an autoimmune condition or disease in a subject. In some embodiments, the subject has or is at risk of developing an autoimmune condition or disease. In some embodiments, the autoimmune condition or disease is selected from type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, and celiac disease.

In some embodiments, the autoimmune disease is type 1 diabetes mellitus. In some embodiments, the autoimmune disease is multiple sclerosis. In some embodiments, the autoimmune disease is systemic lupus erythematosus. In some embodiments, the autoimmune disease is myasthenia gravis. In some embodiments, the autoimmune disease is rheumatoid arthritis. In some embodiments, the autoimmune disease is early onset rheumatoid arthritis. In some embodiments, the autoimmune disease is ankylosing spondylitis. In some embodiments, the autoimmune disease is immune-mediated pregnancy loss. In some embodiments, the autoimmune disease is immune-mediated recurrent pregnancy loss. In some embodiments, the autoimmune disease is dermatomyositis. In some embodiments, the autoimmune disease is psoriatic arthritis. In some embodiments, the autoimmune disease is Crohn's disease. In some embodiments, the autoimmune disease is inflammatory bowel disease (IBD). In some embodiments, the autoimmune disease is ulcerative colitis. In some embodiments, the autoimmune disease is bullous pemphigoid. In some embodiments, the autoimmune disease is pemphigus vulgaris. In some embodiments, the autoimmune disease is autoimmune hepatitis. In some embodiments, the autoimmune disease is psoriasis. In some embodiments, the autoimmune disease is Sjogren's syndrome. In some embodiments, the autoimmune disease is celiac disease.

In some embodiments, the cell expresses an antigen-specific receptor (e.g., T cell receptor or chimeric antigen receptor) that is specific to an antigen associated with the autoimmune disease.

Allergic diseases

In some embodiments, a cell, vector, nucleic acid, or lipid nanoparticle is used for treating, preventing, treating, inhibiting, or ameliorating an allergic condition or disease in a subject. In some embodiments, the subject has or is at risk of developing an allergic condition or disease. In some embodiments, the allergic condition or disease is selected from allergic asthma, steroid-resistant asthma, atopic dermatitis, celiac disease, pollen allergy, food allergy, drug hypersensitivity, and contact dermatitis. In some embodiments, the allergic disease is allergic asthma. In some embodiments, the allergic disease is steroid-resistant asthma. In some embodiments, the allergic disease is atopic dermatitis. In some embodiments, the allergic disease is celiac disease. In some embodiments, the allergic disease is pollen allergy. In some embodiments, the allergic disease is food allergy. In some embodiments, the allergic disease is drug hypersensitivity. In some embodiments, the allergic disease is contact dermatitis.

In some embodiments, the cell expresses an antigen-specific receptor (e.g., T cell receptor or chimeric antigen receptor) that is specific to an antigen associated with the allergic disease.

### Administration

In some embodiments, a cell, vector, nucleic acid, or lipid nanoparticle may be administered between 1 and 14 days over a 30-day period. In some embodiments, doses may be provided 1, 2, 3, 4, 5, 6, 7, 8, 9, 10, 11, 12, 13, or 14 days over a 60-day period. Alternate protocols may be appropriate for individual subjects. A suitable dose is an amount of a compound that, when administered as described above, is capable of detectably altering or ameliorating symptoms, or decreases at least one indicator of autoimmune, allergic or other inflammatory immune activity in a statistically significant manner by at least 10-50% relative to the basal (e.g., untreated) level, which can be monitored by measuring specific levels of blood components, for example, detectable levels of circulating immunocytes and/or other inflammatory cells and/or soluble inflammatory mediators including proinflammatory cytokines.

In some embodiments, rapamycin or a rapalog is administered to the subject before the administration of cells, in conjunction with cells, and/or following the administration of cells. Administration of rapamycin or a rapalog that is capable of inducing dimerization of the CISC components on the surface of a cell results in continued IL-2 signal transduction in vivo, promoting survival and proliferation of the CISC-expressing cell without the undesired effects that would be caused by IL-2 administration, such as activation of other T cells. In some embodiments, the rapamycin or rapalog that is administered is everolimus, CCl-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, and AP23573, or a metabolite or derivative thereof. In some embodiments, the rapamycin or rapalog is administered at a dose of 0.001 mg/kg to 10 mg/kg body mass of the subject, or a dose between 0.001 mg/kg and 10 mg/kg. In some embodiments, the rapamycin or rapalog is administered at a dose of 0.001 mg/kg to 0.01 mg/kg, 0.01 mg/kg to 0.1 mg/kg. 0.1 mg/kg to 1 mg/kg, or 1 mg/kg to 10 mg/kg. In some embodiments, the rapamycin or rapalog is administered in a separate composition from the cells. In some embodiments, the rapamycin or rapalog is administered in multiple doses. In some embodiments, the rapamycin or rapalog is administered for 1, 2, 3, 4, 5, 6, 7, 8, 9, 10, 11, 12, 13, or 14 or more days. In some embodiments, the rapamycin or rapalog is administered for 1, 2, 3, 4, 5, 6, 7, 8, 9, 10, 11, 12, 13, 14, 15, 16, 17, 18, 19, or 20 or more weeks. In some embodiments, the subject is a human. In some embodiments, the administration of the rapamycin or rapalog results in prolonged survival of the administered cells, relative to a subject that is not administered rapamycin or a rapalog. In some embodiments, the administration of the rapamycin or rapalog increases the frequency of cells circulating in the peripheral blood of a subject, relative to a subject that is not administered rapamycin or a rapalog.

In general, an appropriate dosage and treatment regimen provides the cells, vectors, nucleic acids, or lipid nanoparticles in an amount sufficient to provide therapeutic and/or prophylactic benefit. Such a response can be monitored by establishing an improved clinical outcome (e.g., more frequent remissions, complete or partial, or longer disease-free survival) in treated subjects as compared to non-treated subjects. Decreases (e.g., reductions having statistical significance when compared to a relevant control) in preexisting immune responses to an antigen associated with an autoimmune, allergic, or other inflammatory condition as provided herein generally correlate with an improved clinical outcome. Such immune responses may generally be evaluated using standard leukocyte and/or lymphocyte cell surface marker or cytokine expression, proliferation, cytotoxicity or released cytokine assays, which are routine in the art and may be performed using samples obtained from a subject before and after therapy.

In some embodiments, the subject is a human. In some embodiments, the subject is an animal. In some embodiments, the animal is a research animal. In some embodiments, the animal is a domesticated animal. In some embodiments, the animal is a rodent. In some embodiments, the rodent is a mouse, rat, guinea pig, chinchilla, or hamster. In some embodiments, the animal is a dog, cat, rabbit, guinea pig, hamster, or ferret. In some embodiments, the animal is a bovine, swine, llama, alpaca, sheep, or goat.

### EXAMPLES

### Example 1: Generation, In Vitro Characterization, and Phenotyping of Engineered Tregs by Induction or Upregulation of the TGF-β Pathway

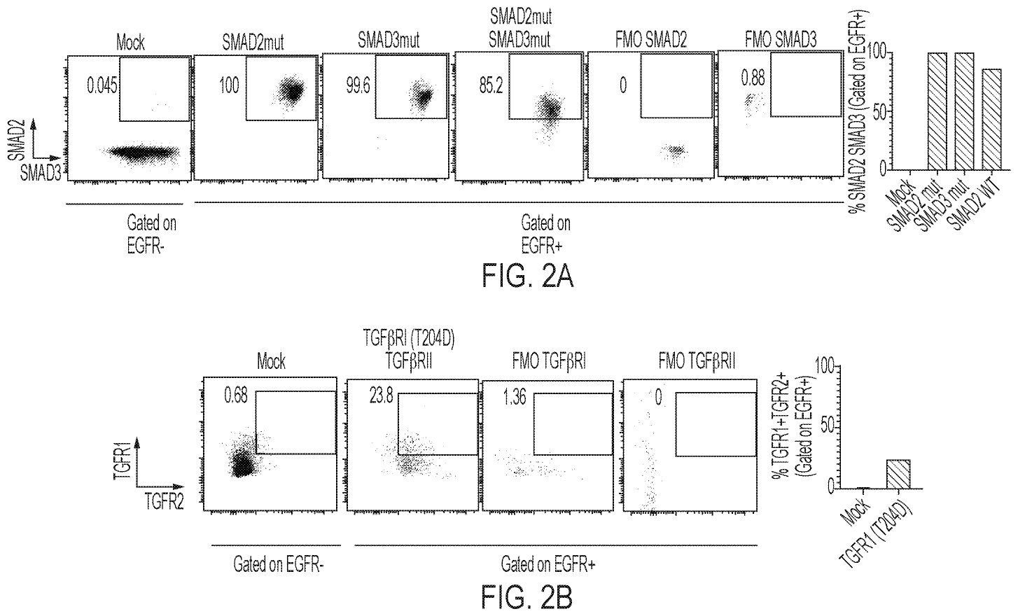

Engineered Tregs were generated through transduction of a lentiviral vector (LVV) encoding constitutively active Smad2 (SMAD2mut), constitutively active Smad3 (SMAD3mut), and/or a constitutively active TGF-β receptor I (TGFβRI (T204D)) with wild-type TGF-β receptor II. CD4+ primary T cells were (i) activated for 2 days with anti-CD 3/anti-CD28 bead stimulation, and (ii) transduced with LVV at a range of MOIs through spinoculation and overnight incubation at 37° C. in 5% CO₂. Culture medium was then replaced with complete medium supplemented with IL-2 for remaining days prior to analysis. All constructs were tagged with a truncated EGFR domain for expression detection, and an antibody detecting EGFR was utilized to measure initial transduction rates. Additional antibodies detecting exogenous proteins of Smad2, Smad3, TGF-β receptor I, and TGF-β receptor II were used to confirm overexpression of the transduced proteins. Initial transduction rates for (a) SMAD2mut; (b) SMAD3mut; (c) SMAD2mut and SMAD3mut; and (d) TGFβRI (T204D)+ TGFβRII constructs were 74.3%, 74.9%, 75.8%, and 18.2%, respectively, as detected by EGFR staining (FIG. 1). Initial transduction rates for SMAD2mut, SMAD3mut, and SMAD2mut+ SMAD3mut were 100%, 99.6%, and 85.2%, respectively, as detected by antibodies specific for Smad2 and Smad3 (FIG. 2A). Initial transduction rates for TGFβRI (T204D) were 23.8% as detected by antibodies specific for TGFβRI and TGFβRII (FIG. 2B).

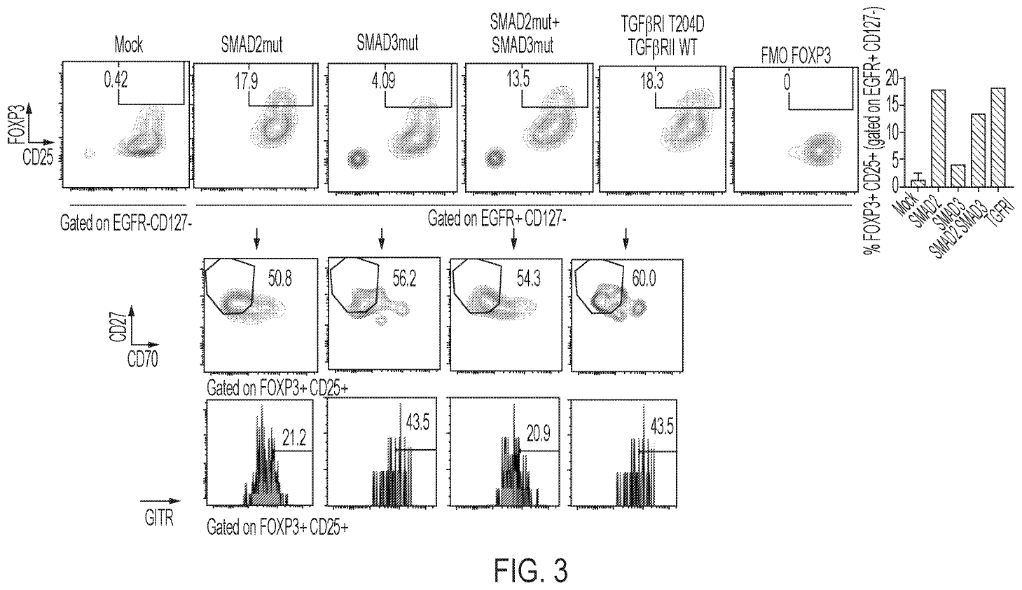

Treg phenotype was evaluated through expression of CD25, FoxP3, CD127 expression in addition to other Treg associated markers including GITR, CD27, and CD70. Engineered Tregs expressing constitutively active Smad2 demonstrated increased Treg phenotype as measured by 17.9% of SMAD2mut-transduced cells being CD25+ FoxP3+CD127-, compared to 0.42% of controls (mock or GFP LVV-transduced) (FIG. 3). Engineered Tregs expressing constitutively active Smad3 demonstrated increased Treg phenotype as measured by 4.09% of SMAD3mut-transduced cells being CD25+ FoxP3+CD127-, compared to 0.42% of controls (mock or GFP LVV-transduced) (FIG. 3). Engineered Tregs expressing TGF-βRI-T204D demonstrated increased Treg phenotype as measured by 18.3% of TGF-βRI-T204D-transduced cells being CD25+ FoxP3+CD127-, compared to 0.42% of controls (mock or GFP LVV-transduced) (FIG. 3).

Within CD25+ FoxP3+CD127-populations, the subsets of CD25+ FoxP3+CD127 -CD27^{hi}CD70^{lo} cells (demonstrating a stable Treg phenotype) represented 50.8%, 56.2%, 54.3%, and 60.0% of CD25+ FoxP3+CD127-cells following transduction with vectors encoding (a) SMAD2mut, (b) SMAD3mut, (c) SMAD2mut+ SMAD3mut, or (d) TGFβRI (T204D)+ TGFβRII, respectively (FIG. 3).

Within CD25+ FoxP3+CD127-populations, the subsets of CD25+ FoxP3+CD127 -GITR+ cells (demonstrating a functional Treg phenotype) represented 21.2%, 43.5%, 20.9%, and 43.5% of CD25+ FoxP3+CD127-cells following transduction with vectors encoding (a) SMAD2mut, (b) SMAD3mut, (c) SMAD2mut+ SMAD3mut, or (d) TGFβRI (T204D)+ TGFβRII, respectively (FIG. 3).

### Example 2: Generation, In Vivo Efficacy, and Phenotyping of Engineered Tregs by Induction or Upregulation of the TGF-β Pathway

Engineered Tregs are generated through transduction of lentiviral vector (LVV) encoding constitutively active Smad2 (SMAD2mut) or Smad3 (SMAD3mut) or a constitutively active TGF-β receptor (TGFβR1 (T204D)) with or without stable MND-FOXP3. CD4+ primary T cells are (i) activated for 2 days with anti-CD3/anti-CD28 bead stimulation, and (ii) transduced with LVV at a range of MOIs through spinoculation and overnight incubation at 37° C. in 5% CO₂. Culture medium is then replaced with complete medium supplemented with IL-2 for remaining days prior to analysis.

Engineered Treg cells are tested for suppression capabilities and Treg phenotype in vivo in a xenogeneic graft-versus-house disease (GvHD) model. Human T effector cells are injected into minimally irradiated NOD.Cg-Prkdc^{scid} Il2^{rgtmlWjl}/SzJ (NSG™) mouse, to an immune response against host tissues, leading to GvHD. Following GvHD induction, engineered Tregs are injected into the mice, and mice are scored for GvHD symptoms, being euthanized if body weight loss exceeds 20%. Mice that receive only human T effector cells demonstrate decreased survival and increased inflammation, compared to mice that are administered engineered Tregs following GvHD induction. Tissues are sampled at one or more timepoints over the duration of disease, to evaluate phenotypes of the engineered Tregs. Treg phenotype is evaluated by measuring expression of CD25, FoxP3, and CD127, quantifying the percentages of cells expressing or lacking each combination of markers, in addition to other Treg-associated markers (e.g., CTLA-4 and ICOS). Engineered Tregs demonstrate a Treg phenotype characterized by FoxP3+, CD25+, CTLA-4+, ICOS+, and/or CD127-(e.g., FoxP3+CD25+CD127-, and optionally ICOS+ and/or CTLA-4+).

### REFERENCES

1. Chipuk et al., J Biol. Chem. 2002. 277 (2): 1240-1248.
2. Funaba and Mathews, Mol Endocrinol. 2000. 14 (10): 1583-1591.
3. Hwang et al., Nat Commun. 2016. 7:10789.
4. Martinez et al., J Biol. Chem. 2009. 284 (51): 35283-35286,
5. Ruetz et al., Cell Stem Cell. 2017. 21 (6): 791-805.
6, Tone et al., Nat Immunol. 2008. 9 (2): 194-202.

All references are incorporated herein by reference in their entirety.

### SEQUENCES

TABLE 1
Examples of nucleic acid sequences

| SEQ ID | | |
|---|---|---|
| NO: | Description | Nucleic acid sequence |
| 1 | Wild-type | ATGGAGGCGGCGGTCGCTGCTCCGCGTCCCCGGCTGCTCCTCCTCGTGCTGGCGG |
| | TGFβRI CDNA | CGGCGGCGGCGGCGGCGGCGGCGCTGCTCCCGGGGGCGACGGCGTTACAGTGTTT |
| | | CTGCCACCTCTGTACAAAAGACAATTTTACTTGTGTGACAGATGGGCTCTGCTTT |
| | | GTCTCTGTCACAGAGACCACAGACAAAGTTATACACAACAGCATGTGTATAGCTG |
| | | AAATTGACTTAATTCCTCGAGATAGGCCGTTTGTATGTGCACCCTCTTCAAAAAC |
| | | TGGGTCTGTGACTACAACATATTGCTGCAATCAGGACCATTGCAATAAAATAGAA |
| | | CTTCCAACTACTGTAAAGTCATCACCTGGCCTTGGTCCTGTGGAACTGGCAGCTG |
| | | TCATTGCTGGACCAGTGTGCTTCGTCTGCATCTCACTCATGTTGATGGTCTATAT |
| | | CTGCCACAACCGCACTGTCATTCACCATCGAGTGCCAAATGAAGAGGACCCTTCA |
| | | TTAGATCGCCCTTTTATTTCAGAGGGTACTACGTTGAAAGACTTAATTTATGATA |
| | | TGACAACGTCAGGTTCTGGCTCAGGTTTACCATTGCTTGTTCAGAGAACAATTGC |
| | | GAGAACTATTGTGTTACAAGAAAGCATTGGCAAAGGTCGATTTGGAGAAGTTTGG |
| | | AGAGGAAAGTGGCGGGGAGAAGAAGTTGCTGTTAAGATATTCTCCTCTAGAGAAG |
| | | AACGTTCGTGGTTCCGTGAGGCAGAGATTTATCAAACTGTAATGTTACGTCATGA |
| | | AAACATCCTGGGATTTATAGCAGCAGACAATAAAGACAATGGTACTTGGACTCAG |
| | | CTCTGGTTGGTGTCAGATTATCATGAGCATGGATCCCTTTTTGATTACTTAAACA |
| | | GATACACAGTTACTGTGGAAGGAATGATAAAACTTGCTCTGTCCACGGCGAGCGG |
| | | TCTTGCCCATCTTCACATGGAGATTGTTGGTACCCAAGGAAAGCCAGCCATTGCT |
| | | CATAGAGATTTGAAATCAAAGAATATCTTGGTAAAGAAGAATGGAACTTGCTGTA |
| | | TTGCAGACTTAGGACTGGCAGTAAGACATGATTCAGCCACAGATACCATTGATAT |
| | | TGCTCCAAACCACAGAGTGGGAACAAAAAGGTACATGGCCCCTGAAGTTCTCGAT |
| | | GATTCCATAAATATGAAACATTTTGAATCCTTCAAACGTGCTGACATCTATGCAA |
| | | TGGGCTTAGTATTCTGGGAAATTGCTCGACGATGTTCCATTGGTGGAATTCATGA |
| | | AGATTACCAACTGCCTTATTATGATCTTGTACCTTCTGACCCATCAGTTGAAGAA |
| | | ATGAGAAAAGTTGTTTGTGAACAGAAGTTAAGGCCAAATATCCCAAACAGATGGC |
| | | AGAGCTGTGAAGCCTTGAGAGTAATGGCTAAAATTATGAGAGAATGTTGGTATGC |
| | | CAATGGAGCAGCTAGGCTTACAGCATTGCGGATTAAGAAAACATTATCGCAACTC |
| | | AGTCAACAGGAAGGCATCAAAATG |
| 2 | TGFβRI- | ATGGAGGCGGCGGTCGCTGCTCCGCGTCCCCGGCTGCTCCTCCTCGTGCTGGCGG |
| | T204D CDNA | CGGCGGCGGCGGCGGCGGCGGCGCTGCTCCCGGGGGCGACGGCGTTACAGTGTTT |
| | (T204D | CTGCCACCTCTGTACAAAAGACAATTTTACTTGTGTGACAGATGGGCTCTGCTTT |
| | codon | GTCTCTGTCACAGAGACCACAGACAAAGTTATACACAACAGCATGTGTATAGCTG |
| | substitution | AAATTGACTTAATTCCTCGAGATAGGCCGTTTGTATGTGCACCCTCTTCAAAAAC |
| | in bold | TGGGTCTGTGACTACAACATATTGCTGCAATCAGGACCATTGCAATAAAATAGAA |
| | underline) | CTTCCAACTACTGTAAAGTCATCACCTGGCCTTGGTCCTGTGGAACTGGCAGCTG |
| | | TCATTGCTGGACCAGTGTGCTTCGTCTGCATCTCACTCATGTTGATGGTCTATAT |
| | | CTGCCACAACCGCACTGTCATTCACCATCGAGTGCCAAATGAAGAGGACCCTTCA |
| | | TTAGATCGCCCTTTTATTTCAGAGGGTACTACGTTGAAAGACTTAATTTATGATA |
| | | TGACAACGTCAGGTTCTGGCTCAGGITTACCATTGCTTGTTCAGAGAACAATTGC |
| | | GAGAGATATTGTGTTACAAGAAAGCATTGGCAAAGGTCGATTTGGAGAAGTTTGG |
| | | AGAGGAAAGTGGCGGGGAGAAGAAGTTGCTGTTAAGATATTCTCCTCTAGAGAAG |
| | | AACGTTCGTGGTTCCGTGAGGCAGAGATTTATCAAACTGTAATGTTACGTCATGA |
| | | AAACATCCTGGGATTTATAGCAGCAGACAATAAAGACAATGGTACTTGGACTCAG |
| | | CTCTGGTTGGTGTCAGATTATCATGAGCATGGATCCCTTTTTGATTACTTAAACA |
| | | GATACACAGTTACTGTGGAAGGAATGATAAAACTTGCTCTGTCCACGGCGAGCGG |
| | | TCTTGCCCATCTTCACATGGAGATTGTTGGTACCCAAGGAAAGCCAGCCATTGCT |
| | | CATAGAGATTTGAAATCAAAGAATATCTTGGTAAAGAAGAATGGAACTTGCTGTA |
| | | TTGCAGACTTAGGACTGGCAGTAAGACATGATTCAGCCACAGATACCATTGATAT |
| | | TGCTCCAAACCACAGAGTGGGAACAAAAAGGTACATGGCCCCTGAAGTTCTCGAT |
| | | GATTCCATAAATATGAAACATTTTGAATCCTTCAAACGTGCTGACATCTATGCAA |
| | | TGGGCTTAGTATTCTGGGAAATTGCTCGACGATGTTCCATTGGTGGAATTCATGA |
| | | AGATTACCAACTGCCTTATTATGATCTTGTACCTTCTGACCCATCAGTTGAAGAA |
| | | ATGAGAAAAGTTGTTTGTGAACAGAAGTTAAGGCCAAATATCCCAAACAGATGGC |
| | | AGAGCTGTGAAGCCTTGAGAGTAATGGCTAAAATTATGAGAGAATGTTGGTATGC |
| | | CAATGGAGCAGCTAGGCTTACAGCATTGCGGATTAAGAAAACATTATCGCAACTC |
| | | AGTCAACAGGAAGGCATCAAAATG |
| 3 | TGFβRI- | ATGGAAGCAGCAGTGGCAGCTCCTAGACCCAGGCTCTTGCTTCTGGTTCTGGCAG |
| | T204D CDNA | CCGCTGCTGCAGCTGCTGCCGCATTGCTGCCAGGAGCAACAGCTCTGCAATGTTT |
| | (codon- | CTGTCACCTGTGTACCAAGGACAATTTCACATGCGTGACAGACGGACTGTGCTTC |
| | optimized) | GTCAGTGTGACTGAGACCACCGACAAAGTCATTCATAACTCCATGTGTATTGCAG |
| | (T204D | AGATCGATCTCATTCCACGCGATAGACCCTTTGTGTGTGCTCCAAGTTCTAAGAC |
| | codon | CGGCTCCGTTACCACAACATACTGCTGTAACCAGGATCACTGCAATAAAATTGAA |
| | substitution | CTTCCCACTACAGTCAAATCATCTCCAGGACTGGGTCCCGTCGAACTTGCTGCAG |
| | in bold | TGATTGCAGGACCCGTCTGTTTCGTGTGTATCAGCCTGATGCTCATGGTTTATAT |
| | underline) | TTGTCACAATCGCACCGTGATTCACCATCGCGTCCCTAATGAAGAGGACCCATCC |
| | | CTCGATAGGCCATTCATCTCGGAGGGTACAACCCTGAAAGACCTGATTTATGATA |
| | | TGACTACTTCTGGATCTGGTAGTGGGTTGCCCCTTCTCGTGCAGAGGACTATCGC |
| | | ACGGGATATCGTGCTGCAAGAATCTATCGGTAAGGGACGGTTCGGTGAGGTCTGG |
| | | AGAGGAAAGTGGAGGGGTGAAGAGGTTGCAGTCAAGATCTTTTCTTCCAGGGAAG |
| | | AACGGAGCTGGTTCAGAGAGGCTGAAATCTATCAGACTGTCATGCTTAGACACGA |
| | | GAACATCCTGGGCTTTATTGCCGCCGACAATAAAGATAATGGCACCTGGACCCAA |
| | | CTGTGGTTGGTGTCTGACTATCACGAACATGGGAGCCTGTTCGATTATTTGAATA |
| | | GGTACACAGTGACTGTCGAGGGCATGATTAAACTGGCTCTGAGCACAGCTAGCGG |
| | | CCTTGCTCACTTGCACATGGAGATCGTCGGGACCCAAGGGAAGCCTGCGATTGCC |
| | | CACAGAGATCTGAAATCCAAGAATATACTGGTGAAGAAAAATGGGACTTGTTGCA |
| | | TTGCTGACTTGGGTCTGGCGGTGAGACATGACTCAGCCACAGATACCATTGACAT |
| | | CGCCCCAAACCACAGGGTTGGGACAAAGAGGTACATGGCCCCTGAGGTCCTTGAC |
| | | GACTCCATTAATATGAAACATTTCGAGTCCTTCAAGCGCGCGGATATTTATGCTA |
| | | TGGGACTTGTGTTTTGGGAGATTGCCAGACGGTGTTCAATCGGAGGGATTCATGA |
| | | GGACTACCAGTTGCCCTATTACGACCTGGTGCCTAGTGATCCCTCTGTGGAGGAG |
| | | ATGAGAAAGGTGGTCTGTGAACAGAAGCTCAGACCTAATATCCCCAACCGGTGGC |
| | | AGTCATGCGAAGCCCTGAGAGTGATGGCCAAAATCATGCGTGAATGCTGGTATGC |
| | | AAATGGTGCCGCTAGACTTACTGCCCTCCGTATTAAAAAAACCCTCAGCCAACTG |
| | | TCTCAGCAAGAGGGAATAAAGATG |
| 4 | Wild-type | ATGTCGTCCATCTTGCCATTCACGCCGCCAGTIGTGAAGAGACTGCTGGGATGGA |
| | SMAD2 CDNA | AGAAGTCAGCTGGTGGGTCTGGAGGAGCAGGCGGAGGAGAGCAGAATGGGCAGGA |
| | | AGAAAAGTGGTGTGAGAAAGCAGTGAAAAGTCTGGTGAAGAAGCTAAAGAAAACA |
| | | GGACGATTAGATGAGCTTGAGAAAGCCATCACCACTCAAAACTGTAATACTAAAT |
| | | GTGTTACCATACCAAGCACTTGCTCTGAAATTTGGGGACTGAGTACACCAAATAC |
| | | GATAGATCAGTGGGATACAACAGGCCTTTACAGCTTCTCTGAACAAACCAGGTCT |
| | | CTTGATGGTCGTCTCCAGGTATCCCATCGAAAAGGATTGCCACATGTTATATATT |
| | | GCCGATTATGGCGCTGGCCTGATCTTCACAGTCATCATGAACTCAAGGCAATTGA |
| | | AAACTGCGAATATGCTTTTAATCTTAAAAAGGATGAAGTATGTGTAAACCCTTAC |
| | | CACTATCAGAGAGTTGAGACACCAGTTTTGCCTCCAGTATTAGTGCCCCGACACA |
| | | CCGAGATCCTAACAGAACTTCCGCCTCTGGATGACTATACTCACTCCATTCCAGA |
| | | AAACACTAACTTCCCAGCAGGAATTGAGCCACAGAGTAATTATATTCCAGAAACG |
| | | CCACCTCCTGGATATATCAGTGAAGATGGAGAAACAAGTGACCAACAGTTGAATC |
| | | AAAGTATGGACACAGGCTCTCCAGCAGAACTATCTCCTACTACTCTTTCCCCTGT |
| | | TAATCATAGCTTGGATTTACAGCCAGTTACTTACTCAGAACCTGCATTTTGGTGT |
| | | TCGATAGCATATTATGAATTAAATCAGAGGGTTGGAGAAACCTTCCATGCATCAC |
| | | AGCCCTCACTCACTGTAGATGGCTTTACAGACCCATCAAATTCAGAGAGGTTCTG |
| | | CTTAGGTTTACTCTCCAATGTTAACCGAAATGCCACGGTAGAAATGACAAGAAGG |
| | | CATATAGGAAGAGGAGTGCGCTTATACTACATAGGTGGGGAAGTTTTTGCTGAGT |
| | | GCCTAAGTGATAGTGCAATCTTTGTGCAGAGCCCCAATTGTAATCAGAGATATGG |
| | | CTGGCACCCTGCAACAGTGTGTAAAATTCCACCAGGCTGTAATCTGAAGATCTTC |
| | | AACAACCAGGAATTTGCTGCTCTTCTGGCTCAGTCTGTTAATCAGGGTTTTGAAG |
| | | CCGTCTATCAGCTAACTAGAATGTGCACCATAAGAATGAGTTTTGTGAAAGGGTG |
| | | GGGAGCAGAATACCGAAGGCAGACGGTAACAAGTACTCCTTGCTGGATTGAACTT |
| | | CATCTGAATGGACCTCTACAGTGGTTGGACAAAGTATTAACTCAGATGGGATCCC |
| | | CTTCAGTGCGTTGCTCAAGCATGTCA |
| 5 | SMAD 2mut | ATGTCGTCCATCTTGCCATTCACGCCGCCAGTIGTGAAGAGACTGCTGGGATGGA |
| | (S464E, S465E, | AGAAGTCAGCTGGTGGGTCTGGAGGAGCAGGCGGAGGAGAGCAGAATGGGCAGGA |
| | S467E) cDNA | AGAAAAGTGGTGTGAGAAAGCAGTGAAAAGTCTGGTGAAGAAGCTAAAGAAAACA |
| | (codon | GGACGATTAGATGAGCTTGAGAAAGCCATCACCACTCAAAACTGTAATACTAAAT |
| | substitutions | GTGTTACCATACCAAGCACTTGCTCTGAAATTTGGGGACTGAGTACACCAAATAC |
| | in bold | GATAGATCAGTGGGATACAACAGGCCTTTACAGCTTCTCTGAACAAACCAGGTCT |
| | underline) | CTTGATGGTCGTCTCCAGGTATCCCATCGAAAAGGATTGCCACATGTTATATATT |
| | | GCCGATTATGGCGCTGGCCTGATCTTCACAGTCATCATGAACTCAAGGCAATTGA |
| | | AAACTGCGAATATGCTTTTAATCTTAAAAAGGATGAAGTATGTGTAAACCCTTAC |
| | | CACTATCAGAGAGTTGAGACACCAGTTTTGCCTCCAGTATTAGTGCCCCGACACA |
| | | CCGAGATCCTAACAGAACTTCCGCCTCTGGATGACTATACTCACTCCATTCCAGA |
| | | AAACACTAACTTCCCAGCAGGAATTGAGCCACAGAGTAATTATATTCCAGAAACG |
| | | CCACCTCCTGGATATATCAGTGAAGATGGAGAAACAAGTGACCAACAGTTGAATC |
| | | AAAGTATGGACACAGGCTCTCCAGCAGAACTATCTCCTACTACTCTTTCCCCTGT |
| | | TAATCATAGCTTGGATTTACAGCCAGTTACTTACTCAGAACCTGCATTTTGGTGT |
| | | TCGATAGCATATTATGAATTAAATCAGAGGGTTGGAGAAACCTTCCATGCATCAC |
| | | AGCCCTCACTCACTGTAGATGGCTTTACAGACCCATCAAATTCAGAGAGGTTCTG |
| | | CTTAGGTTTACTCTCCAATGTTAACCGAAATGCCACGGTAGAAATGACAAGAAGG |
| | | CATATAGGAAGAGGAGTGCGCTTATACTACATAGGTGGGGAAGTTTTTGCTGAGT |
| | | GCCTAAGTGATAGTGCAATCTTTGTGCAGAGCCCCAATTGTAATCAGAGATATGG |
| | | CTGGCACCCTGCAACAGTGTGTAAAATTCCACCAGGCTGTAATCTGAAGATCTTC |
| | | AACAACCAGGAATTTGCTGCTCTTCTGGCTCAGTCTGTTAATCAGGGTTTTGAAG |
| | | CCGTCTATCAGCTAACTAGAATGTGCACCATAAGAATGAGTTTTGTGAAAGGGTG |
| | | GGGAGCAGAATACCGAAGGCAGACGGTAACAAGTACTCCTTGCTGGATTGAACTT |
| | | CATCTGAATGGACCTCTACAGTGGTTGGACAAAGTATTAACTCAGATGGGATCCC |
| | | CTTCAGTGCGTTGCGAAGAGATGGAA |
| 6 | SMAD2mut | ATGAGCTCTATCCTCCCCTTCACACCTCCTGTTGTGAAACGGCTCTTGGGATGGA |
| | (S464E, S465E, | AGAAAAGCGCAGGTGGCTCTGGTGGAGCTGGAGGCGGAGAGCAAAACGGGCAGGA |
| | S467E) cDNA | AGAGAAATGGTGCGAGAAAGCAGTGAAGTCTCTGGTCAAGAAACTCAAGAAGACA |
| | (codon- | GGAAGACTGGACGAACTTGAGAAAGCCATCACAACTCAAAACTGTAATACCAAAT |
| | optimized) | GCGTTACTATCCCCTCAACTTGTTCAGAGATCTGGGGTCTGAGCACACCAAATAC |
| | (codon | CATTGACCAGTGGGACACCACTGGACTGTATAGCTTCAGCGAGCAAACTCGCAGC |
| | substitutions | CTTGATGGAAGGCTCCAAGTGTCCCATAGAAAGGGGCTACCACATGTGATTTACT |
| | in bold | GTCGGCTTTGGAGGTGGCCTGATCTGCACTCTCATCACGAACTGAAGGCGATCGA |
| | underline) | GAACTGCGAATACGCGTTTAACCTCAAGAAAGACGAAGTGTGTGTCAATCCCTAC |
| | | CATTACCAACGTGTTGAGACCCCTGTACTGCCTCCAGTGCTTGTGCCCAGACACA |
| | | CCGAAATTCTGACTGAGTTGCCACCTTTGGATGATTACACACATTCCATCCCCGA |
| | | GAATACTAACTTCCCAGCAGGTATCGAACCTCAGTCCAACTACATCCCCGAAACA |
| | | CCACCTCCTGGATATATCAGCGAAGATGGCGAGACATCTGACCAACAGCTCAATC |
| | | AGAGCATGGATACAGGATCTCCCGCTGAACTCAGCCCTACAACTCTCAGCCCTGT |
| | | TAACCACAGCCTTGATCTGCAACCAGTGACTTATAGTGAACCTGCATTCTGGTGC |
| | | TCCATTGCATACTACGAGCTGAATCAGAGGGTTGGCGAGACATTTCACGCTAGCC |
| | | AACCTTCTCTCACAGTGGACGGTTTTACAGATCCTTCCAATTCCGAAAGGTTCTG |
| | | CCTTGGCCTCTTGTCAAACGTGAACCGGAATGCGACTGTTGAGATGACCAGAAGG |
| | | CACATTGGTCGTGGCGTTCGGCTGTACTACATCGGAGGGGAAGTGTTCGCTGAGT |
| | | GCCTTAGCGACAGTGCGATCTTCGTCCAGAGCCCAAATTGCAACCAGCGCTATGG |
| | | ATGGCACCCAGCGACTGTTTGTAAGATTCCCCCAGGCTGTAATTTGAAGATCTTT |
| | | AATAATCAGGAGTTTGCAGCCCTGTTGGCCCAGAGTGTGAACCAGGGGTTTGAAG |
| | | CGGTGTATCAACTGACTCGCATGTGCACTATTCGGATGAGCTTTGTGAAGGGATG |
| | | GGGTGCCGAGTACAGAAGACAGACTGTTACCTCTACACCTTGTTGGATTGAACTC |
| | | CATCTTAACGGCCCACTGCAATGGCTTGATAAGGTGCTTACACAGATGGGGTCAC |
| | | CATCAGTGCGCTGCGAAGAGATGGAA |
| 7 | Wild-type | ATGTCGTCCATCCTGCCTTTCACTCCCCCGATCGTGAAGCGCCTGCTGGGCTGGA |
| | SMAD3 CDNA | AGAAGGGCGAGCAGAACGGGCAGGAGGAGAAATGGTGCGAGAAGGCGGTCAAGAG |
| | | CCTGGTCAAGAAACTCAAGAAGACGGGGCAGCTGGACGAGCTGGAGAAGGCCATC |
| | | ACCACGCAGAACGTCAACACCAAGTGCATCACCATCCCCAGGTCCCTGGATGGCC |
| | | GGTTGCAGGTGTCCCATCGGAAGGGGCTCCCTCATGTCATCTACTGCCGCCTGTG |
| | | GCGATGGCCAGACCTGCACAGCCACCACGAGCTACGGGCCATGGAGCTGTGTGAG |
| | | TTCGCCTTCAATATGAAGAAGGACGAGGTCTGCGTGAATCCCTACCACTACCAGA |
| | | GAGTAGAGACACCAGTTCTACCTCCTGTGTTGGTGCCACGCCACACAGAGATCCC |
| | | GGCCGAGTTCCCCCCACTGGACGACTACAGCCATTCCATCCCCGAAAACACTAAC |
| | | TTCCCCGCAGGCATCGAGCCCCAGAGCAATATTCCAGAGACCCCACCCCCTGGCT |
| | | ACCTGAGTGAAGATGGAGAAACCAGTGACCACCAGATGAACCACAGCATGGACGC |
| | | AGGTTCTCCAAACCTATCCCCGAATCCGATGTCCCCAGCACATAATAACTTGGAC |
| | | CTGCAGCCAGTTACCTACTGCGAGCCGGCCTTCTGGTGCTCCATCTCCTACTACG |
| | | AGCTGAACCAGCGCGTCGGGGAGACATTCCACGCCTCGCAGCCATCCATGACTGT |
| | | GGATGGCTTCACCGACCCCTCCAATTCGGAGCGCTTCTGCCTAGGGCTGCTCTCC |
| | | AATGTCAACAGGAATGCAGCAGTGGAGCTGACACGGAGACACATCGGAAGAGGCG |
| | | TGCGGCTCTACTACATCGGAGGGGAGGTCTTCGCAGAGTGCCTCAGTGACAGCGC |
| | | TATTTTTGTCCAGTCTCCCAACTGTAACCAGCGCTATGGCTGGCACCCGGCCACC |
| | | GTCTGCAAGATCCCACCAGGATGCAACCTGAAGATCTTCAACAACCAGGAGTTCG |
| | | CTGCCCTCCTGGCCCAGTCGGTCAACCAGGGCTTTGAGGCTGTCTACCAGTTGAC |
| | | CCGAATGTGCACCATCCGCATGAGCTTCGTCAAAGGCTGGGGAGCGGAGTACAGG |
| | | AGACAGACTGTGACCAGTACCCCCTGCTGGATTGAGCTGCACCTGAATGGGCCTT |
| | | TGCAGTGGCTTGACAAGGTCCTCACCCAGATGGGCTCCCCAAGCATCCGCTGTTC |
| | | CAGTGTGTCT |
| 8 | SMAD3 | ATGTCGTCCATCCTGCCTTTCACTCCCCCGATCGTGAAGCGCCTGCTGGGCTGGA |
| | (S422D, S423D, | AGAAGGGCGAGCAGAACGGGCAGGAGGAGAAATGGTGCGAGAAGGCGGTCAAGAG |
| | S425D) CDNA | CCTGGTCAAGAAACTCAAGAAGACGGGGCAGCTGGACGAGCTGGAGAAGGCCATC |
| | (codon | ACCACGCAGAACGTCAACACCAAGTGCATCACCATCCCCAGGTCCCTGGATGGCC |
| | substitutions | GGTTGCAGGTGTCCCATCGGAAGGGGCTCCCTCATGTCATCTACTGCCGCCTGTG |
| | in bold | GCGATGGCCAGACCTGCACAGCCACCACGAGCTACGGGCCATGGAGCTGTGTGAG |
| | underline) | TTCGCCTTCAATATGAAGAAGGACGAGGTCTGCGTGAATCCCTACCACTACCAGA |
| | | GAGTAGAGACACCAGTTCTACCTCCTGTGTTGGTGCCACGCCACACAGAGATCCC |
| | | GGCCGAGTTCCCCCCACTGGACGACTACAGCCATTCCATCCCCGAAAACACTAAC |
| | | TTCCCCGCAGGCATCGAGCCCCAGAGCAATATTCCAGAGACCCCACCCCCTGGCT |
| | | ACCTGAGTGAAGATGGAGAAACCAGTGACCACCAGATGAACCACAGCATGGACGC |
| | | AGGTTCTCCAAACCTATCCCCGAATCCGATGTCCCCAGCACATAATAACTTGGAC |
| | | CTGCAGCCAGTTACCTACTGCGAGCCGGCCTTCTGGTGCTCCATCTCCTACTACG |
| | | AGCTGAACCAGCGCGTCGGGGAGACATTCCACGCCTCGCAGCCATCCATGACTGT |
| | | GGATGGCTTCACCGACCCCTCCAATTCGGAGCGCTTCTGCCTAGGGCTGCTCTCC |
| | | AATGTCAACAGGAATGCAGCAGTGGAGCTGACACGGAGACACATCGGAAGAGGCG |
| | | TGCGGCTCTACTACATCGGAGGGGAGGTCTTCGCAGAGTGCCTCAGTGACAGCGC |
| | | TATTTTTGTCCAGTCTCCCAACTGTAACCAGCGCTATGGCTGGCACCCGGCCACC |
| | | GTCTGCAAGATCCCACCAGGATGCAACCTGAAGATCTTCAACAACCAGGAGTTCG |
| | | CTGCCCTCCTGGCCCAGTCGGTCAACCAGGGCTTTGAGGCTGTCTACCAGTTGAC |
| | | CCGAATGTGCACCATCCGCATGAGCTTCGTCAAAGGCTGGGGAGCGGAGTACAGG |
| | | AGACAGACTGTGACCAGTACCCCCTGCTGGATTGAGCTGCACCTGAATGGGCCTT |
| | | TGCAGTGGCTTGACAAGGTCCTCACCCAGATGGGCTCCCCAAGCATCCGCTGTGA |
| | | TGACGTGGAT |
| 9 | SMAD 3mut | ATGTCCTCCATCTTGCCTTTTACACCACCTATCGTGAAACGCCTTCTGGGTTGGA |
| | (S422D, | AAAAAGGCGAACAAAATGGCCAAGAAGAAAAGTGGTGTGAGAAGGCCGTCAAATC |
| | S423D, | ATTGGTGAAGAAGTTGAAAAAGACAGGACAGCTTGATGAGCTGGAAAAGGCCATT |
| | S425D) cDNA | ACTACCCAGAATGTTAATACTAAGTGCATCACAATCCCACGCTCTCTGGATGGAA |
| | (codon- | GACTGCAAGTGAGCCACAGGAAAGGTTTGCCACATGTCATCTATTGTAGACTCTG |
| | optimized) | GAGATGGCCAGATTTGCATAGTCACCATGAGCTGAGAGCTATGGAGCTTTGTGAA |
| | (codon | TTTGCATTCAATATGAAGAAAGATGAGGTGTGCGTTAATCCCTACCACTACCAGA |
| | substitutions | GAGTGGAAACTCCCGTTCTGCCTCCAGTTCTGGTGCCAAGACACACAGAAATTCC |
| | in bold | GGCAGAATTTCCCCCTCTGGATGATTACAGTCACTCAATCCCTGAGAACACAAAC |
| | underline) | TTCCCTGCTGGGATTGAGCCCCAGTCTAACATCCCCGAAACACCACCACCTGGTT |
| | | ATCTTAGCGAAGACGGCGAAACTTCTGACCATCAGATGAATCACAGCATGGATGC |
| | | TGGCTCTCCCAATCTGAGCCCAAACCCTATGTCACCAGCACACAATAATCTGGAC |
| | | TTGCAGCCTGTCACTTACTGTGAACCTGCTTTTTGGTGCTCTATTAGCTATTACG |
| | | AGCTCAATCAGAGGGTGGGAGAAACCTTTCACGCTAGCCAACCCTCCATGACCGT |
| | | GGATGGGTTCACTGACCCAAGCAACTCTGAAAGATTTTGTCTGGGTCTCTTGAGT |
| | | AACGTGAACAGGAATGCAGCCGTCGAGCTCACACGCAGACATATCGGGAGAGGAG |
| | | TGCGGTTGTACTATATCGGAGGCGAAGTGTTCGCCGAGTGCCTGTCAGATTCTGC |
| | | CATCTTCGTCCAGAGCCCAAACTGTAATCAGAGGTATGGATGGCACCCAGCAACA |
| | | GTGTGTAAAATTCCTCCCGGATGCAACCTCAAGATTTTCAACAATCAGGAGTTTG |
| | | CCGCTCTTCTCGCACAATCCGTGAATCAGGGTTTCGAGGCCGTGTATCAGCTGAC |
| | | TAGAATGTGCACAATCCGGATGAGCTTCGTTAAAGGCTGGGGAGCCGAGTATCGC |
| | | AGGCAAACAGTTACATCAACCCCATGTTGGATCGAGCTCCACCTGAACGGACCAC |
| | | TGCAATGGCTAGACAAGGTCCTGACACAAATGGGTTCCCCAAGTATTCGGTGTGA |
| | | TGACGTGGAT |

TABLE 2
Examples of amino acid sequences

| SEQ ID | | |
|---|---|---|
| NO: | Description | Amino acid sequence |
| 10 | Wild-type TGFβRI | MEAAVAAPRPRLLLLVLAAAAAAAAALLPGATALQCFCHLCTKDNFT |
| | (full-length) | CVTDGLCFVSVTETTDKVIHNSMCIAEIDLIPRDRPFVCAPSSKTGS |
| | | VTTTYCCNQDHCNKIELPTTVKSSPGLGPVELAAVIAGPVCFVCISL |
| | | MLMVYICHNRTVIHHRVPNEEDPSLDRPFISEGTTLKDLIYDMTTSG |
| | | SGSGLPLLVQRTIARTIVLQESIGKGRFGEVWRGKWRGEEVAVKIFS |
| | | SREERSWFREAEIYQTVMLRHENILGFIAADNKDNGTWTQLWLVSDY |
| | | HEHGSLFDYLNRYTVTVEGMIKLALSTASGLAHLHMEIVGTQGKPAI |
| | | AHRDLKSKNILVKKNGTCCIADLGLAVRHDSATDTIDIAPNHRVGTK |
| | | RYMAPEVLDDSINMKHFESFKRADIYAMGLVFWEIARRCSIGGIHED |
| | | YQLPYYDLVPSDPSVEEMRKVVCEQKLRPNIPNRWQSCEALRVMAKI |
| | | MRECWYANGAARLTALRIKKTLSQLSQQEGIKM |
| 11 | TGFβRI-T204D | MEAAVAAPRPRLLLLVLAAAAAAAAALLPGATALQCFCHLCTKDNFT |
| | (full-length) | CVTDGLCFVSVTETTDKVIHNSMCIAEIDLIPRDRPFVCAPSSKTGS |
| | (substitution in | VTTTYCCNQDHCNKIELPTTVKSSPGLGPVELAAVIAGPVCFVCISL |
| | bold underline) | MLMVYICHNRTVIHHRVPNEEDPSLDRPFISEGTTLKDLIYDMTTSG |
| | | SGSGLPLLVQRTIARDIVLQESIGKGREGEVWRGKWRGEEVAVKIFS |
| | | SREERSWFREAEIYQTVMLRHENILGFIAADNKDNGTWTQLWLVSDY |
| | | HEHGSLFDYLNRYTVTVEGMIKLALSTASGLAHLHMEIVGTQGKPAI |
| | | AHRDLKSKNILVKKNGTCCIADLGLAVRHDSATDTIDIAPNHRVGTK |
| | | RYMAPEVLDDSINMKHFESFKRADIYAMGLVFWEIARRCSIGGIHED |
| | | YQLPYYDLVPSDPSVEEMRKVVCEQKLRPNIPNRWQSCEALRVMAKI |
| | | MRECWYANGAARLTALRIKKTLSQLSQQEGIKM |
| 12 | Wild-type TGFβRI | LQCFCHLCTKDNFTCVTDGLCFVSVTETTDKVIHNSMCIAEIDLIPR |
| | (extracellular- | DRPFVCAPSSKTGSVTTTYCCNQDHCNKIELPTTVKSSPGLGPVELA |
| | transmembrane- | AVIAGPVCFVCISLMLMVYICHNRTVIHHRVPNEEDPSLDRPFISEG |
| | cytoplasmic | TTLKDLIYDMTTSGSGSGLPLLVQRTIARTIVLQESIGKGRFGEVWR |
| | domains) | GKWRGEEVAVKIFSSREERSWFREAEIYQTVMLRHENILGFIAADNK |
| | | DNGTWTQLWLVSDYHEHGSLFDYLNRYTVTVEGMIKLALSTASGLAH |
| | | LHMEIVGTQGKPAIAHRDLKSKNILVKKNGTCCIADLGLAVRHDSAT |
| | | DTIDIAPNHRVGTKRYMAPEVLDDSINMKHFESFKRADIYAMGLVFW |
| | | EIARRCSIGGIHEDYQLPYYDLVPSDPSVEEMRKVVCEQKLRPNIPN |
| | | RWQSCEALRVMAKIMRECWYANGAARLTALRIKKTLSQLSQQEGIKM |
| 13 | TGFβRI-T204D | LQCFCHLCTKDNFTCVTDGLCFVSVTETTDKVIHNSMCIAEIDLIPR |
| | (extracellular- | DRPFVCAPSSKTGSVTTTYCCNQDHCNKIELPTTVKSSPGLGPVELA |
| | transmembrane- | AVIAGPVCFVCISLMLMVYICHNRTVIHHRVPNEEDPSLDRPFISEG |
| | cytoplasmic | TTLKDLIYDMTTSGSGSGLPLLVQRTIARDIVLQESIGKGRFGEVWR |
| | domains) | GKWRGEEVAVKIFSSREERSWFREAEIYQTVMLRHENILGFIAADNK |
| | (substitution in | DNGTWTQLWLVSDYHEHGSLFDYLNRYTVTVEGMIKLALSTASGLAH |
| | bold underline) | LHMEIVGTQGKPAIAHRDLKSKNILVKKNGTCCIADLGLAVRHDSAT |
| | | DTIDIAPNHRVGTKRYMAPEVLDDSINMKHFESFKRADIYAMGLVFW |
| | | EIARRCSIGGIHEDYQLPYYDLVPSDPSVEEMRKVVCEQKLRPNIPN |
| | | RWQSCEALRVMAKIMRECWYANGAARLTALRIKKTLSQLSQQEGIKM |
| 14 | TGFβRI signal | MEAAVAAPRPRLLLLVLAAAAAAAAALLPGATA |
| | peptide | |
| 15 | TGFβRI | LQCFCHLCTKDNFTCVTDGLCFVSVTETTDKVIHNSMCIAEIDLIPR |
| | extracellular | DRPFVCAPSSKTGSVTTTYCCNQDHCNKIELPTTVKSSPGLGPVEL |
| | domain | |
| 16 | TGFβRI | AAVIAGPVCFVCISLMLMVYI |
| | transmembrane | |
| | domain | |
| 17 | TGFβRI cytoplasmic | CHNRTVIHHRVPNEEDPSLDRPFISEGTTLKDLIYDMTTSGSGSGLP |
| | domain (wild-type) | LLVQRTIARTIVLQESIGKGRFGEVWRGKWRGEEVAVKIFSSREERS |
| | | WFREAEIYQTVMLRHENILGFIAADNKDNGTWTQLWLVSDYHEHGSL |
| | | FDYLNRYTVTVEGMIKLALSTASGLAHLHMEIVGTQGKPAIAHRDLK |
| | | SKNILVKKNGTCCIADLGLAVRHDSATDTIDIAPNHRVGTKRYMAPE |
| | | VLDDSINMKHFESFKRADIYAMGLVFWEIARRCSIGGIHEDYQLPYY |
| | | DLVPSDPSVEEMRKVVCEQKLRPNIPNRWQSCEALRVMAKIMRECWY |
| | | ANGAARLTALRIKKTLSQLSQQEGIKM |
| 18 | TGFβRI cytoplasmic | CHNRTVIHHRVPNEEDPSLDRPFISEGTTLKDLIYDMTTSGSGSGLP |
| | domain (T204D) | LLVQRTIARDIVLQESIGKGRFGEVWRGKWRGEEVAVKIFSSREERS |
| | (substitution in | WFREAEIYQTVMLRHENILGFIAADNKDNGTWTQLWLVSDYHEHGSL |
| | bold underline) | FDYLNRYTVTVEGMIKLALSTASGLAHLHMEIVGTQGKPAIAHRDLK |
| | | SKNILVKKNGTCCIADLGLAVRHDSATDTIDIAPNHRVGTKRYMAPE |
| | | VLDDSINMKHFESFKRADIYAMGLVFWEIARRCSIGGIHEDYQLPYY |
| | | DLVPSDPSVEEMRKVVCEQKLRPNIPNRWQSCEALRVMAKIMRECWY |
| | | ANGAARLTALRIKKTLSQLSQQEGIKM |
| 19 | Wild-type Smad2 | MSSILPFTPPVVKRLLGWKKSAGGSGGAGGGEQNGQEEKWCEKAVKS |
| | | LVKKLKKTGRLDELEKAITTQNCNTKCVTIPSTCSEIWGLSTPNTID |
| | | QWDTTGLYSFSEQTRSLDGRLQVSHRKGLPHVIYCRLWRWPDLHSHH |
| | | ELKAIENCEYAFNLKKDEVCVNPYHYQRVETPVLPPVLVPRHTEILT |
| | | ELPPLDDYTHSIPENTNFPAGIEPQSNYIPETPPPGYISEDGETSDQ |
| | | QLNQSMDTGSPAELSPTTLSPVNHSLDLQPVTYSEPAFWCSIAYYEL |
| | | NQRVGETFHASQPSLTVDGFTDPSNSERFCLGLLSNVNRNATVEMTR |
| | | RHIGRGVRLYYIGGEVFAECLSDSAIFVQSPNCNQRYGWHPATVCKI |
| | | PPGCNLKIFNNQEFAALLAQSVNQGFEAVYQLTRMCTIRMSFVKGWG |
| | | AEYRRQTVTSTPCWIELHLNGPLQWLDKVLTQMGSPSVRCSSMS |
| 20 | Smad2mut (S464E, | MSSILPFTPPVVKRLLGWKKSAGGSGGAGGGEQNGQEEKWCEKAVKS |
| | S465E, S467E) | LVKKLKKTGRLDELEKAITTQNCNTKCVTIPSTCSEIWGLSTPNTID |
| | (substitutions in | QWDTTGLYSFSEQTRSLDGRLQVSHRKGLPHVIYCRLWRWPDLHSHH |
| | bold underline) | ELKAIENCEYAFNLKKDEVCVNPYHYQRVETPVLPPVLVPRHTEILT |
| | | ELPPLDDYTHSIPENTNFPAGIEPQSNYIPETPPPGYISEDGETSDQ |
| | | QLNQSMDTGSPAELSPTTLSPVNHSLDLQPVTYSEPAFWCSIAYYEL |
| | | NQRVGETFHASQPSLTVDGFTDPSNSERFCLGLLSNVNRNATVEMTR |
| | | RHIGRGVRLYYIGGEVFAECLSDSAIFVQSPNCNQRYGWHPATVCKI |
| | | PPGCNLKIFNNQEFAALLAQSVNQGFEAVYQLTRMCTIRMSFVKGWG |
| | | AEYRRQTVTSTPCWIELHLNGPLQWLDKVLTQMGSPSVRCEEME |
| 21 | Wild-type Smad3 | MSSILPFTPPIVKRLLGWKKGEQNGQEEKWCEKAVKSLVKKLKKTGQ |
| | | LDELEKAITTQNVNTKCITIPRSLDGRLQVSHRKGLPHVIYCRLWRW |
| | | PDLHSHHELRAMELCEFAFNMKKDEVCVNPYHYQRVETPVLPPVLVP |
| | | RHTEIPAEFPPLDDYSHSIPENTNFPAGIEPQSNIPETPPPGYLSED |
| | | GETSDHQMNHSMDAGSPNLSPNPMSPAHNNLDLQPVTYCEPAFWCSI |
| | | SYYELNQRVGETFHASQPSMTVDGFTDPSNSERFCLGLLSNVNRNAA |
| | | VELTRRHIGRGVRLYYIGGEVFAECLSDSAIFVQSPNCNQRYGWHPA |
| | | TVCKIPPGCNLKIFNNQEFAALLAQSVNQGFEAVYQLTRMCTIRMSF |
| | | VKGWGAEYRRQTVTSTPCWIELHLNGPLQWLDKVLTQMGSPSIRCSS |
| | | VS |
| 22 | Smad3mut (S422D, | MSSILPFTPPIVKRLLGWKKGEQNGQEEKWCEKAVKSLVKKLKKTGQ |
| | S423D, S425D) | LDELEKAITTQNVNTKCITIPRSLDGRLQVSHRKGLPHVIYCRLWRW |
| | (substitutions in | PDLHSHHELRAMELCEFAFNMKKDEVCVNPYHYQRVETPVLPPVLVP |
| | bold underline) | RHTEIPAEFPPLDDYSHSIPENTNFPAGIEPQSNIPETPPPGYLSED |
| | | GETSDHQMNHSMDAGSPNLSPNPMSPAHNNLDLQPVTYCEPAFWCSI |
| | | SYYELNQRVGETFHASQPSMTVDGFTDPSNSERFCLGLLSNVNRNAA |
| | | VELTRRHIGRGVRLYYIGGEVFAECLSDSAIFVQSPNCNQRYGWHPA |
| | | TVCKIPPGCNLKIFNNQEFAALLAQSVNQGFEAVYQLTRMCTIRMSF |
| | | VKGWGAEYRRQTVTSTPCWIELHLNGPLQWLDKVLTQMGSPSIRCDD |
| | | VD |
| 23 | TGFβRI kinase | TTSGSGSG |
| | domain | |
| 24 | Smad2 | SSMS |
| | phosphorylation | |
| | motif | |
| 25 | Smad3 | CSSVS |
| | phosphorylation | |
| | motif | |
| 26 | Linker | GGGS |
| 27 | Linker | GGGSGGG |
| 28 | CISC component 1 | EMWHEGLEEASRLYFGERNVKGMFEVLEPLHAMMERGPQTLKE |
| | (FRB | TSFNQAYGRDLMEAQEWCRKYMKSGNVKDLTQAWDLYYHVERR |
| | extracellular | ISKGKDTIPWLGHLLVGLSGAFGFIILVYLLINCRNTGPWLKK |
| | domain-IL2Rβ | VLKCNTPDPSKFFSQLSSEHGGDVQKWLSSPFPSSSFSPGGLA |
| | transmembrane | PEISPLEVLERDKVTQLLLQQDKVPEPASLSSNHSLTSCFTNQ |
| | domain-IL2Rβ | GYFFFHLPDALEIEACQVYFTYDPYSEEDPDEGVAGAPTGSSP |
| | cytoplasmic | QPLQPLSGEDDAYCTFPSRDDLLLFSPSLLGGPSPPSTAPGGS |
| | domain) | GAGEERMPPSLQERVPRDWDPQPLGPPTPGVPDLVDFQPPPEL |
| | | VLREAGEEVPDAGPREGVSFPWSRPPGQGEFRALNARLPLNTD |
| | | AYLSLQELQGQDPTHLV |
| 29 | CISC component 2 | GVQVETISPGDGRTFPKRGQTCVVHYTGMLEDGKKFDSSRDRN |
| | (FKBP | KPFKFMLGKQEVIRGWEEGVAQMSVGQRAKLTISPDYAYGATG |
| | extracellular | HPGIIPPHATLVFDVELLKLGEGSNTSKENPFLFALEAVVISV |
| | domain-IL2Rγ | GSMGLIISLLCVYFWLERIMPRIPTLKNLEDLVTEYHGNFSAW |
| | transmembrane | SGVSKGLAESLQPDYSERLCLVSEIPPKGGALGEGPGASPCNQ |
| | domain-IL2Rγ | HSPYWAPPCYTLKPET |
| | cytoplasmic | |
| | domain) | |
| 30 | Soluble FRB | EMWHEGLEEASRLYFGERNVKGMFEVLEPLHAMMERGPQTLKE |
| | domain | TSFNQAYGRDLMEAQEWCRKYMKSGNVKDLTQAWDLYYHVERR |
| | | ISK |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Additional Embodiments

1. A method for producing engineered Treg cells, the method comprising modifying a cell to promote expression and/or activity of TGFβRI, Smad2, and/or Smad3 protein.
2. The method of embodiment 1, wherein the method results in stabilization of Treg suppressive function and homogenization of a population of cells in which the cell is present.
3. The method of embodiment 1 or 2, wherein the cell is an immune cell that is a CD4+ cell, a CD8+ cell, or a CD3+ cell; or an iPSC.
4. The method of any one of the preceding embodiments, wherein modifying a cell comprises contacting the cell with a nucleic acid vector that encodes a constitutively active
TGFβRI, Smad2 and/or Smad3 protein.
5. The method of embodiment 4, wherein the constitutively active Smad2 or Smad3 comprises one or more amino acid substitutions of one or more C-terminal serines.
6, The method of embodiment 5, wherein constitutively active Smad2 or Smad3 comprises one or more substitutions in a C-terminal Ser-Ser-X-Ser phosphorylation motif.
7. The method of embodiment 6, wherein the one or more substitutions in a C-terminal Ser-Ser-X-Ser phosphorylation motif is Asp-Asp-X-Asp.
8. The method of embodiment 4, wherein the constitutively active SMAD2 comprises one or more amino acid substitutions of Ser464, Ser465, and/or Ser467.
9. The method of any one of embodiments 4-8, wherein the constitutively active
TGFβRI comprises one or more substitutions of amino acids in a GS domain, optionally in the amino acid sequence TTSGSGSG (SEQ ID NO: 23).
10. The method of embodiment 9, wherein the constitutively active TGFβRI comprises a T204D substitution.
11. The method of embodiment 4, wherein the vector is a plasmid or a viral vector.
12. The method of embodiment 11, wherein the viral vector is a recombinant AAV vector or lentiviral vector.
13. The method of any one of the preceding embodiments, wherein modifying a cell comprises editing a TGFβRI, SMAD2, and/or SMAD3 gene of the immune cell to introduce a variant sequence that encodes a constitutively active TGFβRI, Smad2 and/or Smad3 protein, respectively.
14. The method of any one of the preceding embodiments, further comprising inhibiting Smad7 expression and/or activity in the cell.
15. The method of any one of the preceding embodiments, wherein the cell is in vivo.
16, The method of any one of the preceding embodiments, wherein the cell is ex vivo.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of.” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of,” or “exactly one of.” “Consisting essentially of.” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising.” “including.” “carrying.” “having.” “containing.” “involving.” “holding.” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be 5 appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A method of producing a genetically modified cell, the method comprising introducing into the cell a nucleic acid comprising:
a heterologous promoter that is operably linked to a sequence encoding TGFβRI, Smad2, and/or Smad3, or one or more functional derivatives of TGFβRI, Smad2, and/or Smad3.

2. The method of claim 1, wherein the promoter is operably linked to a sequence encoding TGFβRI.

3. The method of claim 1 or 2, wherein the TGFβRI comprises one or more substitutions of amino acids in a GS domain, optionally in the amino acid sequence TTSGSGSG (SEQ ID NO: 23), or at an amino acid corresponding to Thr204, optionally wherein the TGFβRI comprises an aspartate or glutamate at a position corresponding to Thr204, optionally wherein the TGFβRI comprises a T204D substitution.

4. The method of claim 2, wherein the promoter is operably linked to a sequence encoding Smad2 or Smad3.

5. The method of claim 1 or 4, wherein the Smad2 or Smad3 comprises one or more amino acid substitutions of one or more C-terminal serines.

6. The method of any one of claim 1 or 4-5, wherein the Smad2 or Smad3 comprises one or more substitutions in a C-terminal Ser-Ser-X-Ser phosphorylation motif.

7. The method of any one of claims 5-6, wherein each of the serines in the Ser-Ser-X-Ser phosphorylation motif is substituted with an aspartate or glutamate.

8. The method of any one of claim 1 or 4-7, wherein the Smad2 comprises one or more amino acid substitutions of Ser464, Ser465, and/or Ser467.

9. The method of any one of claim 1 or 4-7, wherein the Smad3 comprises one or more amino acid substitutions of Ser422, Ser423, and/or Ser425.

10. The method of any one of claims 1-9, wherein the method further comprises introducing into the cell a nuclease or a nucleic acid encoding the nuclease, wherein the nuclease is capable of cleaving a nucleic acid sequence in the endogenous gene.

11. The method of claim 10, wherein the nuclease is a zinc finger nuclease, TALEN, meganuclease, or RNA-guided DNA endonuclease.

12. The method of claim 10 or 11, wherein the nuclease is an RNA-guided DNA endonuclease, and wherein the method further comprises introducing into the cell a gRNA comprising a spacer sequence that is complementary to a nucleic acid sequence in the endogenous gene.

13. The method of claim 11 or 12, wherein the RNA-guided DNA endonuclease is a Cas endonuclease.

14. The method of claim 13, wherein the Cas endonuclease is a Cas9 endonuclease.

15. A method of producing a genetically modified cell, the method comprising inserting a heterologous promoter into a nucleic acid of a cell genome upstream from a coding sequence of an endogenous TGFβRI, SMAD2, or SMAD3 gene on the nucleic acid, wherein the inserted promoter is operably linked to the endogenous TGFβRI, SMAD2, SMAD3 gene.

16. The method of claim 15, wherein the heterologous promoter is inserted downstream of an endogenous promoter of the endogenous TGFβRI, SMAD2, or SMAD3 gene.

17. The method of claim 15, wherein the heterologous promoter is inserted within an endogenous promoter of the endogenous TGFβRI, SMAD2, or SMAD3 gene, wherein insertion disrupts the endogenous promoter.

18. The method of any one of claims 15-17, wherein the inserted promoter is operably linked to the endogenous TGFβRI gene, and wherein the nucleic acid comprising the heterologous promoter further comprises:
(i) a 5′ homology arm having homology to a first nucleic acid sequence in the endogenous TGFRBI gene; and
(ii) a 3′ homology arm having homology to a second nucleic acid sequence in the endogenous TGFβRI gene that is downstream from the first nucleic acid sequence in the endogenous TGFβRI,
wherein the heterologous promoter is located between 5′ and 3′ homology arms.

19. The method of claim 18, wherein the method further comprises modifying the endogenous TGFβRI gene to produce a modified TGFβRI gene encoding a modified TGFβRI protein, wherein the modified TGFβRI protein comprises one or more substitutions of amino acids in a GS domain, optionally in the amino acid sequence TTSGSGSG (SEQ ID NO: 23), or at an amino acid corresponding to Thr204, optionally wherein the TGFβRI comprises an aspartate or glutamate at a position corresponding to Thr204, optionally wherein the TGFβRI comprises a T204D substitution,
optionally wherein the modifying comprises introducing into the cell a nuclease or a nucleic acid encoding the nuclease, and a modifying template comprising:
(i) a 5′ homology arm having homology to a sequence in the TGFβRI gene upstream from the nucleotide sequence encoding the amino acids of the GS domain;
(ii) a nucleic acid sequence encoding the substituted amino acids of the GS domain; and
(iii) a 3′ homology arm having homology to a sequence in the TGFβRI gene downstream from the nucleotide sequence encoding the amino acids of the GS domain,
wherein the nuclease is capable of cleaving the TGFβRI gene at a position within TGFβRI gene to promoter insertion of the modifying template to produce the modified TGFβRI gene.

20. The method of claim 15, wherein the inserted promoter is operably linked to the endogenous SMAD2 or SMAD3 gene, and wherein the nucleic acid comprising the heterologous promoter further comprises:
(i) a 5′ homology arm having homology to a first nucleic acid sequence in the endogenous SMAD2 or SMAD3 gene; and
(ii) a 3′ homology arm having homology to a second nucleic acid sequence in the same endogenous gene as the 5′ homology arm,
wherein the heterologous promoter is located between 5′ and 3′ homology arms.

21. The method of claim 20, wherein the method further comprises modifying the endogenous SMAD2 or SMAD3 gene to produce a modified SMAD2 or SMAD3 gene encoding a modified SMAD2 or SMAD3 protein, wherein the modified Smad2 or Smad3 protein comprises one or more amino acid substitutions of one or more C-terminal serines.

22. The method of claim 21, wherein the modified Smad2 or Smad3 protein comprises one or more substitutions in a C-terminal Ser-Ser-X-Ser phosphorylation motif.

23. The method of claim 21 or 22, wherein each of the serines in the Ser-Ser-X-Ser phosphorylation motif is substituted with an aspartate or glutamate.

24. The method of any one of claims 21-23, wherein the modified Smad2 protein comprises one or more amino acid substitutions of Ser464, Ser465, and/or Ser467.

25. The method of any one of claims 21-24, wherein the modified Smad3 protein comprises one or more amino acid substitutions of Ser422, Ser424, and/or Ser425.

26. The method of any one of claims 21-25, wherein the modifying comprises introducing into the cell a nuclease or a nucleic acid encoding the nuclease, and a modifying template comprising:
(i) a 5′ homology arm having homology to a sequence in the SMAD2 or SMAD3 gene upstream from the nucleotide sequence encoding the one or more C-terminal serines;
(ii) a nucleic acid sequence encoding the substituted amino acids of the C-terminal serines; and
(iii) a 3′ homology arm having homology to a sequence in the SMAD2 or SMAD3 gene downstream from the nucleotide sequence encoding the one or more C-terminal serines,
wherein the nuclease is capable of cleaving the SMAD2 or SMAD3 gene at a position within the SMAD2 or SMAD3 gene to promoter insertion of the modifying template to produce the modified SMAD2 or SMAD3 gene.

27. The method of any one of claims 15-26, wherein the method further comprises introducing into the cell a nuclease or a nucleic acid encoding the nuclease, wherein the nuclease is capable of cleaving a nucleic acid sequence in a targeted locus.

28. The method of claim 27, wherein the nuclease is a zinc finger nuclease, TALEN, meganuclease, or RNA-guided DNA endonuclease.

29. The method of claim 27 or 28, wherein the nuclease is an RNA-guided DNA endonuclease, and wherein the method further comprises introducing into the cell a gRNA comprising a spacer sequence that is complementary to a nucleic acid sequence in the targeted locus.

30. The method of claim 27 or 28, wherein the RNA-guided DNA endonuclease is a Cas endonuclease.

31. The method of claim 27, wherein the Cas endonuclease is a Cas9 endonuclease.

32. The method of any one of claims 15-31, wherein the targeted locus is a safe harbor locus.

33. The method of claim 32, wherein the safe harbor locus is a HIPP11 locus, ROSA26 locus, or AAVS1 locus.

34. The method of any one of claims 15-31, wherein the targeted locus is a TRAC or TRBC locus.

35. The method of any one of claims 1-34, wherein the promoter is a constitutive promoter.

36. The method of claim 35, wherein the constitutive promoter is an EF-1a, a PGK promoter, or an MND promoter.

37. The method of any one of claims 1-36, wherein the promoter is an MND promoter.

38. The method of any one of claims 1-34, wherein the promoter is an inducible promoter.

39. The method of claim 37, wherein the inducible promoter is inducible by a drug or steroid.

40. The method of any one of claims 1-39, wherein the nucleic acid comprising the heterologous promoter is comprised in a vector.

41. The method of claim 40, wherein the vector is a viral vector.

42. The method of claim 40 or 41, wherein the vector is an adenovirus-associated virus (AAV) vector.

43. The method of claim 42, wherein the AAV vector is derived from an AAV of serotype AAV1, AAV2, AAV3, AAV4, AAV5, AAV6, AAV7, AAV8, AAV9, AAV10, or AAV11.

44. The method of claim 41, wherein the viral vector is a lentiviral vector.

45. The method of claim 40, wherein the vector is a plasmid.

46. The method of claim 40, wherein the vector is a bacterial artificial chromosome.

47. The method of claim 40, wherein the vector is a human artificial chromosome.

48. The method of any one of claim 1-40 or 45-47, wherein the nucleic acid comprising the heterologous promoter is comprised in a lipid nanoparticle.

49. The method of any one of claims 1-48, wherein the method further comprises reducing expression of Smad7.

50. The method of claim 49, wherein reducing expression of Smad7 comprises introducing a SMAD^{null} mutation into at least one SMAD7 allele of the cell genome.

51. The method of claim 49 or 50, wherein reducing expression of Smad7 comprises introducing one or more SMAD7^{null} mutations into each SMAD7 allele of the cell genome.

52. The method of any one of claims 49-51, wherein reducing expression of Smad7 comprises removing one or more exons of a SMAD7 allele from the cell genome.

53. The method of any one of claims 49-52, wherein reducing expression of Smad7 comprises removing one or more exons of each SMAD7 allele from the cell genome.

54. The method of any one of any one of claims 49-53, wherein reducing expression of Smad7 comprises removing each exon of each SMAD7 allele from the cell genome.

55. The method of any one of claims 49-54, wherein reducing Smad7 expression comprises introducing into the cell an RNA interference (RNAi) molecule comprising a nucleic acid sequence that is complementary to a coding sequence encoding SMAD7.

56. The method of claim 55, wherein the RNAi molecule is an miRNA, siRNA, or shRNA.

57. The method of any one of claims 49-56, wherein the cell in which Smad7 expression is reduced does not express detectable Smad7.

58. The method of any one of claims 1-57, wherein the method further comprises reducing expression of IL-6R.

59. The method of claim 58, wherein reducing expression of IL-6R comprises introducing an IL6null mutation into at least one IL6R allele of the cell genome.

60. The method of claim 58 or 59, wherein reducing expression of IL-6R comprises introducing one or more IL6R^{null} mutations into each IL6R allele of the cell genome.

61. The method of any one of claims 58-60, wherein reducing expression of IL-6R comprises removing one or more exons of an IL6R allele from the cell genome.

62. The method of any one of claims 58-61, wherein reducing expression of IL-6R comprises removing one or more exons of each IL6R allele from the cell genome.

63. The method of any one of claims 58-62, wherein reducing expression of IL-6R comprises removing each exon of each IL6R allele from the cell genome.

64. The method of any one of claims 58-63, wherein reducing IL-6R expression comprises introducing into the cell an RNA interference (RNAi) molecule comprising a nucleic acid sequence that is complementary to a coding sequence encoding IL-6R.

65. The method of claim 64, wherein the RNAi molecule is an miRNA, siRNA, or shRNA.

66. The method of any one of claims 58-65, wherein the cell in which IL-6R expression is reduced does not express detectable IL-6R.

67. The method of any one of claims 1-66, wherein the method further comprises reducing expression of gp130.

68. The method of claim 67, wherein reducing expression of gp130 comprises introducing a GP130^{null} mutation into at least one GP130 allele of the cell genome.

69. The method of claim 67 or 68, wherein reducing expression of gp130 comprises introducing one or more GP130^{null} mutations into each GP130 allele of the cell genome.

70. The method of any one of claims 67-69, wherein reducing expression of gp130 comprises removing one or more exons of a GP130 allele from the cell genome.

71. The method of any one of claims 67-70, wherein reducing expression of gp130 comprises removing one or more exons of each GP130 allele from the cell genome.

72. The method of any one of claims 67-71, wherein reducing expression of gp130 comprises removing each exon of each GP130 allele from the cell genome.

73. The method of any one of claims 67-72, wherein reducing gp130 expression comprises introducing into the cell an RNA interference (RNAi) molecule comprising a nucleic acid sequence that is complementary to a coding sequence encoding gp130.

74. The method of claim 73, wherein the RNAi molecule is an miRNA, siRNA, or shRNA.

75. The method of any one of claims 67-74, wherein the cell in which gp130 expression is reduced does not express detectable gp130.

76. A cell made by the method of any one of claims 1-75.

77. A genetically modified cell comprising a heterologous promoter operably linked to a cDNA coding sequence encoding TGFβRI, Smad2, or Smad3.

78. The cell of claim 77, wherein the heterologous promoter and cDNA coding sequence are located in a safe harbor locus.

79. The cell of claim 78, wherein the safe harbor locus is a HIPP11 locus, ROSA26 locus, or AAVS1 locus.

80. The cell of claim 77, wherein the heterologous promoter and cDNA coding sequence are located in a TRAC or TRBC locus.

81. The cell of any one of claims 77-80, wherein the heterologous promoter is operably linked to a sequence encoding TGFβRI.

82. The cell of claim 81, wherein the TGFβRI comprises one or more substitutions of amino acids in a GS domain, optionally in the amino acid sequence TTSGSGSG (SEQ ID NO: 23), or an amino acid corresponding to Thr204, optionally wherein the TGFβRI comprises an aspartate or glutamate at a position corresponding to Thr204, optionally wherein the TGFβRI comprises a T204D substitution.

83. The cell of any one of claims 77-80, wherein the heterologous promoter is operably linked to a coding sequence encoding Smad2 or Smad3.

84. The cell of any one of claim 77-80 or 83, wherein the Smad2 or Smad3 comprises one or more amino acid substitutions of one or more C-terminal serines.

85. The cell of any one of claim 77-80 or 83-84, wherein the Smad2 or Smad3 comprises one or more substitutions in a C-terminal Ser-Ser-X-Ser phosphorylation motif.

86. The cell of claim 84 or 85, wherein each of the serines in the Ser-Ser-X-Ser phosphorylation motif is substituted with an aspartate or glutamate.

87. The cell of any one of claim 77-80 or 83-86, wherein the Smad2 comprises one or more amino acid substitutions of Ser464, Ser465, and/or Ser467.

88. The cell of any one of claim 77-80 or 83-86, wherein the Smad3 comprises one or more amino acid substitutions of Ser422, Ser423, and/or Ser425.

89. A genetically modified cell comprising a heterologous promoter inserted upstream from a coding sequence of an endogenous TGFβRI, SMAD2, or SMAD3 gene on a nucleic acid of the cell genome, wherein the inserted promoter is operably linked to the endogenous TGFβRI, SMAD2, SMAD3 coding sequence.

90. The cell of claim 89, wherein the inserted promoter is operably linked to the endogenous TGFβRI gene.

91. The cell of claim 89 or 90, wherein the endogenous TGFβRI gene is modified to produce a modified TGFβRI gene encoding a modified TGFβRI, wherein the modified TGFβRI comprises one or more substitutions of amino acids in a GS domain, optionally in the amino acid sequence TTSGSGSG (SEQ ID NO: 23), or at an amino acid corresponding to Thr204, optionally wherein the TGFβRI comprises an aspartate or glutamate at a position corresponding to Thr204, optionally wherein the TGFβRI comprises a T204D substitution.

92. The cell of claim 89, wherein the heterologous promoter is operably linked to the endogenous SMAD2 or SMAD3 gene.

93. The cell of claim 89 or 92, wherein the endogenous SMAD2 or SMAD3 gene is modified to produce a modified SMAD2 or SMAD3 gene encoding a modified Smad2 or Smad3, wherein the modified Smad2 or Smad3 comprises one or more amino acid substitutions of one or more C-terminal serines.

94. The cell of claim 93, wherein the modified Smad2 or Smad3 comprises one or more substitutions in a C-terminal Ser-Ser-X-Ser phosphorylation motif.

95. The cell of claim 93 or 94, wherein each of the serines in the Ser-Ser-X-Ser phosphorylation motif is substituted with an aspartate or glutamate.

96. The cell of any one of claims 93-95, wherein the modified Smad2 comprises one or more amino acid substitutions of Ser464, Ser465, and/or Ser467.

97. The cell of any one of claims 93-95, wherein the modified Smad3 comprises one or more amino acid substitutions of Ser422, Ser423, and/or Ser425.

98. The cell of any one of claims 77-97, wherein the promoter is a constitutive promoter.

99. The cell of claim 98, wherein the constitutive promoter is an EF-1a, a PGK promoter, or an MND promoter.

100. The cell of claim 99, wherein the promoter is an MND promoter.

101. The cell of any one of claims 77-97, wherein the promoter is an inducible promoter.

102. The cell of claim 101, wherein the inducible promoter is inducible by a drug or steroid.

103. The cell of any one of claims 77-102, wherein the cell comprises a SMAD7^{null} mutation in at least one SMAD7 allele of the cell genome.

104. The cell of any one of claims 77-103, wherein the cell comprises a SMAD7^{null} mutation in each SMAD7 allele of the cell genome.

105. The cell of any one of claims 77-104, wherein the cell comprises a SMAD7 knockout allele.

106. The cell of any one of claims 77-105, wherein the cell is homozygous for a SMAD7 knockout allele.

107. The cell of any one of claims 77-106, wherein the cell does not express detectable Smad7.

108. The cell of any one of claims 77-107, wherein the cell comprises a IL6R^{null} mutation in at least one IL6R allele of the cell genome.

109. The cell of any one of claims 77-108, wherein the cell comprises a IL6R^{null} mutation in each IL6R allele of the cell genome.

110. The cell of any one of claims 77-109, wherein the cell comprises a IL6R knockout allele.

111. The cell of any one of claims 77-110, wherein the cell is homozygous for a IL6R knockout allele.

112. The cell of any one of claims 77-111, wherein the cell does not express detectable IL-6R.

113. The cell of any one of claims 77-112, wherein the cell comprises a GP130^{null} mutation in at least one GP130 allele of the cell genome.

114. The cell of any one of claims 77-113, wherein the cell comprises a GP130null mutation in each GP130 allele of the cell genome.

115. The cell of any one of claims 77-114, wherein the cell comprises a GP130 knockout allele.

116. The cell of any one of claims 77-115, wherein the cell is homozygous for a GP130 knockout allele.

117. The cell of any one of claims 77-116, wherein the cell does not express detectable gp130.

118. The cell of any one of claims 76-117, wherein the cell is a stem cell or T cell.

119. The cell of any one of claims 76-118, wherein the cell is a CD3+, CD4+, or CD8+ T cell.

120. The cell of any one of claims 76-119, wherein the cell is a Treg cell.

121. The cell of any one of claims 76-120, wherein the cell is a FoxP3+ Treg cell.

122. The cell of any one of claims 76-121, wherein the cell is CTLA-4+, LAG-3+, CD25+, CD39+, CD27+, CD70+, GITR+, neuropilin-1+, galectin-1+, and/or IL-2Rα+.

123. A pharmaceutical composition comprising the cell of any one of claims 76-122.

124. A method comprising administering the cell of any one of claims 76-122 or the pharmaceutical composition of claim 123 to a subject.

125. The method of claim 124, wherein the subject has or is at risk of developing an inflammatory disease, autoimmune disease, allergic disease, or a condition associated with a solid organ transplant.

126. The method of claim 125, wherein the inflammatory disease is selected from pancreatic islet cell transplantation, asthma, hepatitis, traumatic brain injury, primary sclerosing cholangitis, primary biliary cholangitis, polymyositis, stroke, Still's disease, acute respiratory distress syndrome (ARDS), uveitis, inflammatory bowel disease (IBD), ulcerative colitis, graft-versus-host disease (GvHD), tolerance induction for transplantation, transplant rejection, or sepsis.

127. The method of claim 125, wherein the autoimmune disease is type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, or celiac disease.

128. The method of claim 125, wherein the allergic disease is allergic asthma, steroid-resistant asthma, atopic dermatitis, celiac disease, pollen allergy, food allergy, drug hypersensitivity, or contact dermatitis.

129. The method of claim 125, wherein the condition associated with a solid organ transplant is graft-versus-host disease.

130. The method of claim 124 or 125, wherein the subject has or is at risk of developing type 1 diabetes.

131. The method of claim 124 or 125, wherein the subject has or is at risk of developing inflammatory bowel disease.

132. The method of claim 124 or 125, wherein the subject has or is at risk of developing multiple sclerosis.

133. The method of claim 124 or 125, wherein the subject has or is at risk of developing primary biliary cholangitis.

134. The method of claim 124 or 125, wherein the subject has or is at risk of developing acute respiratory distress syndrome.

135. The method of claim 124 or 125, wherein the subject has or is at risk of developing stroke.

136. The method of claim 124 or 125, wherein the subject has or is at risk of developing graft-versus-host disease.

137. The method of any one of claims 124-136, wherein the cell is autologous to the subject.

138. The method of any one of claims 124-136, wherein the cell is an allogeneic cell.

139. A nucleic acid comprising a promoter that is operably linked to a coding sequence encoding TGFβRI, Smad2, and/or Smad3, or one or more functional derivatives of TGFβRI, Smad2, and/or Smad3.

140. The nucleic acid of claim 139, wherein the coding sequence is a cDNA coding sequence that does not comprise an intron.

141. The nucleic acid of claim 139 or 140, wherein the nucleic acid further comprises:
(i) a 5′ homology arm having homology to a sequence in a targeted locus in a human genome; and
(ii) a 3′ homology arm having homology to a sequence in the same targeted locus as the 5′ homology arm,

142. The nucleic acid of any one of claims 139-141, wherein the promoter and coding sequence are located between the 5′ and 3′ homology arms.

143. The nucleic acid of any one of claims 139-142, wherein the coding sequence encodes TGFβRI or a functional derivative thereof.

144. The nucleic acid of claim 143, wherein the TGFβRI comprises one or more substitutions of amino acids in a GS domain, optionally in the amino acid sequence TTSGSGSG (SEQ ID NO: 23), or at an amino acid corresponding to Thr204, optionally wherein the TGFβRI comprises an aspartate or glutamate at a position corresponding to Thr204, optionally wherein the TGFβRI comprises a T204D substitution.

145. The nucleic acid of any one of claims 139-142, wherein the coding sequence encodes Smad2, Smad3, or a functional derivative thereof.

146. The nucleic acid of any one of claim 139-142 or 145, wherein the Smad2 or Smad3 comprises one or more amino acid substitutions of one or more C-terminal serines.

147. The nucleic acid of any one of claim 139-142 or 145-146, wherein the Smad2 or SMAD3 comprises one or more substitutions in a C-terminal Ser-Ser-X-Ser phosphorylation motif.

148. The nucleic acid of claim 146 or 147, wherein each of the serines in the Ser-Ser-X-Ser phosphorylation motif is substituted with an aspartate or glutamate.

149. The nucleic acid of any one of claims 146-148, wherein the Smad2 comprises one or more amino acid substitutions of Ser464, Ser465, and/or Ser467.

150. The nucleic acid of any one of claims 146-148, wherein the Smad3 comprises one or more amino acid substitutions of Ser422, Ser423, and/or Ser425.

151. The nucleic acid of any one of claims 139-150, wherein the targeted locus is a safe harbor locus.

152. The nucleic acid of claim 151, wherein the safe harbor locus is a HIPP11 or AAVS1 locus.

153. The nucleic acid of any one of claims 139-150, wherein the targeted locus is a TRAC or TRBC locus.

154. The nucleic acid of any one of claims 139-150, wherein the promoter is a constitutive promoter.

155. The nucleic acid of claim 154, wherein the constitutive promoter is an EF-1a, a PGK promoter, or an MND promoter.

156. The nucleic acid of claim 155, wherein the promoter is an MND promoter.

157. The nucleic acid of any one of claims 139-153, wherein the promoter is an inducible promoter.

158. The nucleic acid of claim 157, wherein the inducible promoter is inducible by a drug or steroid.

159. A vector comprising the nucleic acid of any one of claims 139-158.

160. The vector of claim 159, wherein the vector is a viral vector.

161. The vector of claim 159 or 160, wherein the vector is an adenovirus-associated virus (AAV) vector.

162. The vector of claim 161, wherein the AAV vector is derived from an AAV of serotype AAV1, AAV2, AAV3, AAV4, AAV5, AAV6, AAV7, AAV8, AAV9, AAV10, or AAV11.

163. The vector of claim 162, wherein the viral vector is a lentiviral vector.

164. The vector of claim 159, wherein the vector is a plasmid.

165. The vector of claim 159, wherein the vector is a bacterial artificial chromosome.

166. The vector of claim 159, wherein the vector is a human artificial chromosome.

167. The vector of any one of claims 159-166, wherein the vector further comprises a nucleic acid sequence encoding an RNAi molecule comprising a sequence that is complementary to a sequence within a coding sequence encoding Smad7, IL-6R, or gp130.

168. The vector of claim 167, wherein the RNAi molecule is an miRNA, shRNA, or siRNA.

169. A lipid nanoparticle comprising the nucleic acid of any one of claims 139-158 or the vector of any one of claims 159-168.

170. The lipid nanoparticle of claim 169, further comprising an RNAi molecule comprising a sequence that is complementary to a sequence within a coding sequence encoding Smad7, IL-6R, or gp130.

171. The lipid nanoparticle of claim 170, wherein the RNAi molecule is an miRNA, shRNA, or siRNA.

172. A system comprising the nucleic acid of any one of claims 139-158, the vector of any one of claims 159-168, or lipid nanoparticle of any one of claims 169-171, and a nuclease or a nucleic acid encoding the nuclease, wherein the nuclease is capable of cleaving a nucleic acid sequence in the targeted locus.

173. The system of claim 172, wherein the nuclease is a zinc finger DNA endonuclease, TALEN, meganuclease, or RNA-guided DNA endonuclease.

174. The system of claim 172 or 173, wherein the nuclease is an RNA-guided DNA endonuclease.

175. The system of claim 172 or 173, wherein the RNA-guided DNA endonuclease is a Cas endonuclease.

176. The system of claim 175, wherein the Cas endonuclease is a Cas9 endonuclease.

177. A pharmaceutical composition comprising the nucleic acid of any one of claims 139-158, vector of any one of claims 159-168, lipid nanoparticle of any one of claims 169-171, or system of any one of claims 172-176; and a pharmaceutically acceptable excipient.

178. A method comprising administering the nucleic acid of any one of claims 139-158, vector of any one of claims 159-168, lipid nanoparticle of any one of claims 169-171, system of any one of claims 172-176, or the pharmaceutical composition of claim 177, to a subject.

179. The method of claim 178, wherein the subject has or is at risk of developing an inflammatory disease, autoimmune disease, allergic disease, or a condition associated with a solid organ transplant.

180. The method of claim 179, wherein the inflammatory disease is selected from pancreatic islet cell transplantation, asthma, hepatitis, traumatic brain injury, primary sclerosing cholangitis, primary biliary cholangitis, polymyositis, stroke, Still's disease, acute respiratory distress syndrome (ARDS), uveitis, inflammatory bowel disease (IBD), ulcerative colitis, graft-versus-host disease (GvHD), tolerance induction for transplantation, transplant rejection, or sepsis.

181. The method of claim 179, wherein the autoimmune disease is type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, or celiac disease.

182. The method of claim 179, wherein the allergic disease is allergic asthma, steroid-resistant asthma, atopic dermatitis, celiac disease, pollen allergy, food allergy, drug hypersensitivity, or contact dermatitis.

183. The method of claim 179, wherein the condition associated with a solid organ transplant is graft-versus-host disease.

184. The method of claim 178 or 179, wherein the subject has or is at risk of developing type 1 diabetes.

185. The method of claim 178 or 179, wherein the subject has or is at risk of developing inflammatory bowel disease.

186. The method of claim 178 or 179, wherein the subject has or is at risk of developing multiple sclerosis.

187. The method of claim 178 or 179, wherein the subject has or is at risk of developing primary biliary cholangitis.

188. The method of claim 178 or 179, wherein the subject has or is at risk of developing acute respiratory distress syndrome.

189. The method of claim 178 or 179, wherein the subject has or is at risk of developing stroke.

190. The method of claim 178 or 179, wherein the subject has or is at risk of developing graft-versus-host disease.
